SEQUENCE LISTING (4 sequences)

SEQ ID NO: 1 - protein, 1335 aa, Homo sapiens
MESEMLQSPLLGLGEEDEADLTDWNLPLAFMKKRHCEKIEGSKSLAQSWRMKDRMKTVSVALVLCLNVGVDPPDVVKTTPCARLECWIDPLSMGPQKALETIGANLQKQYENWQPRARYKQSLDPTVDEVKKLCTSLRRNAKEERVLFHYNGHGVPRPTVNGEVWVFNKNYTQYIPLSIYDLQTWMGSPSIFVYDCSNAGLIVKSFKQFALQREQELEVAAINPNHPLAQMPLPPSMKNCIQLAACEATELLPMIPDLPADLFTSCLTTPIKIALRWFCMQKCVSLVPGVTLDLIEKIPGRLNDRRTPLGELNWIFTAITDTIAWNVLPRDLFQKLFRQDLLVASLFRNFLLAERIMRSYNCTPVSSPRLPPTYMHAMWQAWDLAVDICLSQLPTIIEEGTAFRHSPFFAEQLTAFQVWLTMGVENRNPPEQLPIVLQVLLSQVHRLRALDLLGRFLDLGPWAVSLALSVGIFPYVLKLLQSSARELRPLLVFIWAKILAVDSSCQADLVKDNGHKYFLSVLADPYMPAEHRTMTAFILAVIVNSYHTGQEACLQGNLIAICLEQLNDPHPLLRQWVAICLGRIWQNFDSARWCGVRDSAHEKLYSLLSDPIPEVRCAAVFALGTFVGNSAERTDHSTTIDHNVAMMLAQLVSDGSPMVRKELVVALSHLVVQYESNFCTVALQFIEEEKNYALPSPATTEGGSLTPVRDSPCTPRLRSVSSYGNIRAVATARSLNKSLQNLSLTEESGGAVAFSPGNLSTSSSASSTLGSPENEEHILSFETIDKMRRASSYSSLNSLIGVSFNSVYTQIWRVLLHLAADPYPEVSDVAMKVLNSIAYKATVNARPQRVLDTSSLTQSAPASPTNKGVHIHQAGGSPPASSTSSSSLTNDVAKQPVSRDLPSGRPGTTGPAGAQYTPHSHQFPRTRKMFDKGPEQTADDADDAAGHKSFISATVQTGFCDWSARYFAQPVMKIPEEHDLESQIRKEREWRFLRNSRVRRQAQQVIQKGITRLDDQIFLNRNPGVPSVVKFHPFTPCIAVADKDSICFWDWEKGEKLDYFHNGNPRYTRVTAMEYLNGQDCSLLLTATDDGAIRVWKNFADLEKNPEMVTAWQGLSDMLPTTRGAGMVVDWEQETGLLMSSGDVRIVRIWDTDREMKVQDIPTGADSCVTSLSCDSHRSLIVAGLGDGSIRVYDRRMALSECRVMTYREHTAWVVKASLQKRPDGHIVSVSVNGDVRIFDPRMPESVNVLQIVKGLTALDIHPQADLIACGSVNQFTAIYNSSGELINNIKYYDGFMGQRVGAISCLAFHPHWPHLAVGSNDYYISVYSVEKRVR

SEQ ID NO: 2 - protein, 1323 aa, Homo sapiens
MKRNGSRNCLNRRSRFGSRERDWLREDVKRGCVYLYGADTTTATTTTTTSSSSSSSSSSSDLHLVLCTVETPASEICAGEGRESLYLQLHGDLVRRLEPTERPLQIVYDYLSRLGFDDPVRIQEEATNPDLGCMIRFYGEKPCHMDRLDRILLSGIYNVRKGKTQLHKWAERLVVLCGTCLIVSSVKDCQTGKMHILPLVGGKIEEVKRRQYSLAFSSAGAQAQTYHVSFETLAEYQRWQRQASKVVSQRISTVDLSCYSLEEVPEHLFYSQDITYLNLRHNFMQLERPGGLDTLYKFSQLKGLNLSHNKLGLFPILLCEISTLTELNLSCNGFHDLPSQIGNLLNLQTLCLDGNFLTTLPEELGNLQQLSSLGISFNNFSQIPEVYEKLTMLDRVVMAGNCLEVLNLGVLNRMNHIKHVDLRMNHLKTMVIENLEGNKHITHVDLRDNRLTDLDLSSLCSLEQLHCGRNQLRELTLSGFSLRTLYASSNRLTAVNVYPVPSLLTFLDLSRNLLECVPDWACEAKKIEVLDVSYNLLTEVPVRILSSLSLRKLMLGHNHVQNLPTLVEHIPLEVLDLQHNALTRLPDTLFSKALNLRYLNASANSLESLPSACTGEESLSMLQLLYLTNNLLTDQCIPVLVGHLHLRILHLANNQLQTFPASKLNKLEQLEELNLSGNKLKTIPTTIANCKRLHTLVAHSNNISIFPEILQLPQIQFVDLSCNDLTEILIPEALPATLQDLDLTGNTNLVLEHKTLDIFSHITTLKIDQKPLPTTDSTVTSTFWSHGLAEMAGQRNKLCVSALAMDSFAEGVGAVYGMFDGDRNEELPRLLQCTMADVLLEEVQQSTNDTVFMANTFLVSHRKLGMAGQKLGSSALLCYIRPDTADPASSFSLTVANVGTCQAVLCRGGKPVPLSKVFSLEQDPEEAQRVKDQKAIITEDNKVNGVTCCTRMLGCTYLYPWILPKPHISSTPLTIQDELLILGNKALWEHLSYTEAVNAVRHVQDPLAAAKKLCTLAQSYGCQDNVGAMVVYLNIGEEGCTCEMNGLTLPGPVGFASTTTIKDAPKPATPSSSSGIASEFSSEMSTSEVSSEVGSTASDEHNAGGLDTALLPRPERRCSLHPTPTSGLFQRQPSSATFSSNQSDNGLDSDDDQPVEGVITNGSKVEVEVDIHCCRGRDLENSPPLIESSPTLCSEEHARGSCFGIRRQNSVNSGMLLPMSKDRMELQKSPSTSCLYGKKLSNGSIVPLEDSLNLIEVATEVPKRKTGYFAAPTQMEPEDQFVVPHDLEEEVKEQMKQHQDSRLEPEPHEEDRTEPPEEFDTAL

SEQ ID NO: 3 - DNA, 1294 bp, Homo sapiens
gcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttctttaccattgtct

SEQ ID NO: 4 - DNA, 2034 bp, Homo sapiens
ggatctggcagcgccgcgaagacgagcggtcaccggcgcccgacccgagcgcgcccagaggacggcggggagccaagccgacccccgagcagcgccgcgcgggccctgaggctcaaaggggcagcttcaggggaggacaccccactggccaggacgccccaggctctgctgctctgccactcagctgccctcggaggagcgtacacacacaccaggactgcattgccccagtgtgcagcccctgccagatgtgggaggcagctagctgcccagaggcatgcccccctgccagccacagcgacccctgctgctgttgctgctgctgctggcctgccagccacaggtcccctccgctcaggtgatggacttcctgtttgagaagtggaagctctacggtgaccagtgtcaccacaacctgagcctgctgccccctcccacggagctggtgtgcaacagaaccttcgacaagtattcctgctggccggacacccccgccaataccacggccaacatctcctgcccctggtacctgccttggcaccacaaagtgcaacaccgcttcgtgttcaagagatgcgggcccgacggtcagtgggtgcgtggaccccgggggcagccttggcgtgatgcctcccagtgccagatggatggcgaggagattgaggtccagaaggaggtggccaagatgtacagcagcttccaggtgatgtacacagtgggctacagcctgtccctgggggccctgctcctcgccttggccatcctggggggcctcagcaagctgcactgcacccgcaatgccatccacgcgaatctgtttgcgtccttcgtgctgaaagccagctccgtgctggtcattgatgggctgctcaggacccgctacagccagaaaattggcgacgacctcagtgtcagcacctggctcagtgatggagcggtggctggctgccgtgtggccgcggtgttcatgcaatatggcatcgtggccaactactgctggctgctggtggagggcctgtacctgcacaacctgctgggcctggccaccctccccgagaggagcttcttcagcctctacctgggcatcggctggggtgcccccatgctgttcgtcgtcccctgggcagtggtcaagtgtctgttcgagaacgtccagtgctggaccagcaatgacaacatgggcttctggtggatcctgcggttccccgtcttcctggccatcctgatcaacttcttcatcttcgtccgcatcgttcagctgctcgtggccaagctgcgggcacggcagatgcaccacacagactacaagttccggctggccaagtccacgctgaccctcatccctctgctgggcgtccacgaagtggtctttgccttcgtgacggacgagcacgcccagggcaccctgcgctccgccaagctcttcttcgacctcttcctcagctccttccagggcctgctggtggctgtcctctactgcttcctcaacaaggaggtgcagtcggagctgcggcggcgttggcaccgctggcgcctgggcaaagtgctatgggaggagcggaacaccagcaaccacagggcctcatcttcgcccggccacggccctcccagcaaggagctgcagtttgggaggggtggtggcagccaggattcatctgcggagacccccttggctggtggcctccctagattggctgagagccccttctgaaccctgctgggaccccagctagggctggactctggcacccagaggcgtcgctggacaacccagaactggacgcccagctgaggctgggggcgggggagccaacagcagcccccacctaccccccacccccagtgtggctgtctgcgagattgggcctcctctccctgcacctgccttgtccctggtgcagaggtgagcagaggagtccagggcgggagtgggggctgtgccgtgaactgcgtgccagtgtccccacgtatgtcggcacgtcccatgtgcatggaaatgtcctccaacaataaagagctcaagtggtcaccgtg

(19) United States
(12) Patent Application Publication
(10) Pub. No.: US 2017/0014477 A1
(43) Pub. Date: Jan. 19, 2017

(54) FREE RAPTOR REDUCES AGING- AND OBESITY-INDUCED FATTY LIVER

(71) Applicants: Utpal PAJVANI, Leonia, NJ (US); KyeongJin KIM, New York, NY (US)

(72) Inventors: Utpal PAJVANI, Leonia, NJ (US); KyeongJin KIM, New York, NY (US)

(73) Assignee: The Trustees of Columbia University in the City of New York, New York, NY (US)

(21) Appl. No.: 15/143,305

(22) Filed: Apr. 29, 2016

Related U.S. Application Data
(60) Provisional application No. 62/267,632, filed on Dec. 15, 2015.
(60) Provisional application No. 62/234,505, filed on Sep. 29, 2015.
(60) Provisional application No. 62/155,696, filed on May 1, 2015.

(51) Int. Cl. A61K 38/17; G01N 33/68; C12N 15/113

(52) U.S. Cl. CPC A61K 38/1709; C12N 15/1138; G01N 33/6827; G01N 33/6893; A61K 48/00

(57) ABSTRACT

The present invention provides a method of reducing a subject's hepatic and plasma triglyceride levels comprising administering to a subject in need thereof a pharmaceutical composition comprising a pharmaceutical carrier and a compound that increases PHLPP2 in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that increases free Raptor in liver cells in an amount effective to reduce the subject's hepatic and plasma triglyceride levels.

The present invention also provides a process for determining the amount of free Raptor in a subject's liver comprising:
a) obtaining a biological sample comprising liver cells of the subject;
separating free Raptor and mTORC1-associated Raptor in the sample; and
c) determining the amount of free Raptor in the sample.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride

This application claims the benefit of U.S. Provisional Application No. 62/267,632, filed Dec. 15, 2015, U.S. Provisional Application No. 62/234,505, filed Sep. 29, 2015, and U.S. Provisional Application No. 62/155,696, filed May 1, 2015, the contents of which are hereby incorporated by reference.

This invention was made with government support under grant number DK093604 awarded by the National Institutes of Health. The government has certain rights in the invention.

This application incorporates-by-reference nucleotide and/or amino acid sequences which are present in the file named “160429_0575_87361_A_SequenceListing_MW.txt,” which is 28 kilobytes in size, and which was created Apr. 29, 2016 in the IBM-PC machine format, having an operating system compatibility with MS-Windows, which is contained in the text file filed Apr. 29, 2016 as part of this application.

Throughout this application various publications are referred to by first author and year of publication. Full citations of these references can be found following the Examples. The disclosures of these publications in their entireties are hereby incorporated by reference into this application in order to more fully describe the state of the art to which this invention pertains.

### BACKGROUND OF THE INVENTION

Obesity has reached epidemic status in the United States—the Centers for Disease Control has stated that more than ⅓ of American adults are obese, and estimated the medical costs attributable to obesity at $147 billion in 2008, a number that is likely to be significantly higher today and in the future.

Obesity-induced metabolic dysfunction manifests as multiple chronic medical conditions, including Type 2 Diabetes (T2D) and Nonalcoholic fatty liver disease (NAFLD) (Ford et al., 2002). NAFLD is the result of compensatory hyperinsulinemia and hepatic de novo lipogenesis. NAFLD contributes to the overall cardiovascular risk of obesity (Villanova et al., 2005), but is also the most common chronic liver disease, predisposing to cirrhosis and hepatocellular carcinoma (Dowman et al., 2011).

Obesity leads to insulin resistance, which begets the hyperglycemia of T2D. In a parallel but poorly understood process, compensatory hyperinsulinemia drives hepatic de novo lipogenesis, mediated in part by the nutrient-sensitive mechanistic target of rapamycin (mTOR) pathway, which predisposes excessive liver fat or NAFLD. The presence of NAFLD increases underlying insulin resistance—this vicious cycle results in exacerbations of both T2D and NAFLD, which show independent associations with cardiovascular disease and all-cause mortality.

Aging and obesity are well-established risk factors for NAFLD (Slawik and Vidal-Puig, 2006), but the molecular mechanism underlying this risk is poorly defined, precluding specific pharmacologic strategies to target excess hepatic triglycerides (TG).

There is no approved pharmacologic therapy for NAFLD—the only clinical recourse is liver transplantation; current projections suggest that NAFLD will be the leading cause for liver transplantation by 2020, a conundrum as available organs are already limiting. Novel pathways are sought to both further our understanding of the pathophysiology, as well as provide new pharmacologic targets to assist in out management of obesity-related morbidity and mortality.

Thus, new therapies are needed.

### SUMMARY OF THE INVENTION

The present invention provides a method of reducing a subject's hepatic and plasma triglyceride levels comprising administering to a subject in need thereof a pharmaceutical composition comprising a pharmaceutical carrier and a compound that increases PHLPP2 in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that increases free Raptor in liver cells in an amount effective to reduce the subject's hepatic and plasma triglyceride levels.

The present invention also provides a process for determining the amount of free Raptor in a subject's liver comprising:
a) obtaining a biological sample comprising liver cells of the subject;
b) separating free Raptor and mTORC1-associated Raptor in the sample; and
c) determining the amount of free Raptor in the sample.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

### BRIEF DESCRIPTION OF THE DRAWINGS

FIG. 1A. Free Raptor levels decline in aged and obese liver. Western blot from liver of young (8-week-old), adult (24-week-old), and ob/ob male mice, following size-exclusion chromatography.

FIG. 1B. Free Raptor levels decline in aged and obese liver. Western blot from liver of young (8-week-old), adult (24-week-old), and ob/ob male mice, following quantification of signal per fraction, normalized to total protein.

FIG. 1C. Western blot from liver of young, adult and ob/ob male mice, cross-linked with disuccinimidyl suberate (DSS).

FIG. 1D. Quantification of signal from western blot (FIG. 1C), as a percentage of control (DMSO-treated liver lysate).

FIG. 1E. Western blot from primary hepatocytes deprived of amino acids or treated with rapamycin, prior to DSS crosslinking.

FIG. 1F. Western blot from primary hepatocytes deprived of amino acids or treated with insulin, prior to DSS crosslinking.

FIG. 2A. mTORC1-independent Raptor in liver is reduced by insulin resistance. Western blots from liver of young (8 week-old) or adult (24 week-old) male mice, showing the whole blot corresponding to FIG. 1C.

FIG. 2B. Western blots from liver of normal chow diet (NCD) or high-fat diet (HFD)-fed male mice, crosslinked with DSS.

FIG. 2C. Quantitation of signal from western blot (FIG. 2B), as a percentage of control (DMSO-treated liver lysate).

FIG. 2D. Western blots from liver of lean or ob/ob mice following immunoprecipitation (IP) with anti-mTOR, with prior crosslinking with dithiobis[succinimidyl propionate] (DSP).

FIG. 2E. Western blots from liver of lean or ob/ob mice following immunoprecipitation (IP) with anti-mTOR, without prior crosslinking with dithiobis[succinimidyl propionate] (DSP).

FIG. 2F. Western blot from primary hepatocytes deprived of amino acids (-AA) or treated with rapamycin (Rapa) for 1 h prior to IP with anti-mTOR.

FIG. 2G. Western blot from Hepa1c1c7 cells, treated with varying concentrations of insulin for 24 hours, prior to DSS crosslinking.

FIG. 3A. Rescue of free Raptor reduces liver weight and triglycerides (TG), without affecting body weight or adiposity. Western blot from liver of aged (10- to 12-month-old) male mice transduced with Ad-GFP or Ad-Raptor, sacrificed after a 16 h fast followed by 4 h refeeding.

FIG. 3B. Western blot from liver of Ad-GFP or Ad-Raptor mice, following size-exclusion chromatography. Fractions 25 and 26 correspond to mTORC1-associated (^{˜}800 kDa) Raptor, while 34 and 35 to free (^{˜}150 kDa) Raptor.

FIG. 3C. Body weight in aged Ad-GFP or Ad-Raptor mice sacrificed after a 16 h fast followed by 4 h refeeding (n=7 per each group).

FIG. 3D. Epididymal WAT (eWAT) weight in aged Ad-GFP or Ad-Raptor mice sacrificed after a 16 h fast followed by 4 h refeeding (n=7 per each group).

FIG. 3E. Liver weight in aged Ad-GFP or Ad-Raptor mice sacrificed after a 16 h fast followed by 4 h refeeding (n=7 per each group). *P<0.05 compared to the indicated control

FIG. 3F. Body weight in adult, high-fat diet (HFD)-fed Ad-GFP or Ad-Raptor mice sacrificed after a 5 h fast (n=5 or 4 per each group).

FIG. 3G. eWAT weight in adult, high-fat diet (HFD)-fed Ad-GFP or Ad-Raptor mice sacrificed after a 5 h fast (n=5 or 4 per each group).

FIG. 3H. Liver weight in adult, high-fat diet (HFD)-fed Ad-GFP or Ad-Raptor mice sacrificed after a 5 h fast (n=5 or 4 per each group).

FIG. 4A. Rescue of free Raptor prevents aging- and obesity-dependent hepatic steatosis. Hepatic lipids in aged (10- to 12-month-old) mice transduced with control (Ad-GFP) or Ad-Raptor, sacrificed after a 16 h fast followed by 4 h refeeding (n=7/group).

FIG. 4B. Hepatic lipids in Ad-GFP or Ad-Raptor diet-induced obese (DIO) mice, sacrificed after a 5 h fast (n=4-5/group).

FIG. 4C. Hepatic lipids in either young (8-week-old) or adult (24-week-old) Ad-GFP or Ad-Raptor mice, sacrificed after a 16 h fast followed by 4 h refeeding (n=6/group).

FIG. 4D. Plasma lipids in aged Ad-GFP or Ad-Raptor mice. *P<0.05, **P<0.01 compared to the indicated control.

FIG. 4E. Plasma lipids in young and adult Ad-GFP or Ad-Raptor mice. *P<0.05, **P<0.01 compared to the indicated control.

FIG. 4F. Hepatic fatty acid synthesis in young or adult Ad-GFP or Ad-Raptor mice, sacrificed after a 16 h fast followed by 4 h refeeding (n=5-6/group). *P<0.05, **P<0.01 compared to the indicated control.

FIG. 4G. Lipogenic gene expression in young or adult Ad-GFP or Ad-Raptor mice, sacrificed after a 16 h fast followed by 4 h refeeding (n=5-6/group). *P<0.05, **P<0.01 compared to the indicated control.

FIG. 5A. Free Raptor prevents aging-induced lipogenesis. (A) Hepatic triglyceride content in young (8 week-old), adult (24week-old) or aged (10- to 12-month-old) male mice sacrificed after a 16 h fast followed by 4 h refeeding (n=6 per each group).

FIG. 5B. Liver weight in young or adult Ad-GFP or Ad-Raptor mice sacrificed after a 16 h fast followed by 4 h refeeding (n=6 per each group). *P<0.05 and **P<0.01 as compared to the indicated control.

FIG. 5C. Western blot of liver protein in young or adult Ad-GFP or Ad-Raptor mice sacrificed after a 16 h fast followed by 4 h refeeding (n=6 per each group).

FIG. 5D. Body weight in young or adult Ad-GFP or Ad-Raptor mice sacrificed after a 16 h fast followed by 4 h refeeding (n=6 per each group).

FIG. 5E. NEFA levels in young or adult Ad-GFP or Ad-Raptor mice sacrificed after a 16 h fast followed by 4 h refeeding (n=6 per each group).

FIG. 5F. Plasma cholesterol levels in young or adult Ad-GFP or Ad-Raptor mice sacrificed after a 16 h fast followed by 4 h refeeding (n=6 per each group).

FIG. 5G. Liver mRNA expression of indicated genes in young or adult Ad-GFP or Ad-Raptor mice sacrificed after a 16 h fast followed by 4 h refeeding (n=6 per each group).

FIG. 5H. Expression of lipogenic genes in primary hepatocytes transduced with Ad-GFP or Ad-Raptor (n=4 biological replicates). *P<0.05 and **P<0.01 as compared to the indicated control.

FIG. 6A. Increase in free Raptor levels does not affect mTORC1 or mTORC2 activity. Western blot from liver proteins from aged Ad-GFP or Ad-Raptor male mice, sacrificed after a 16 h fast followed by 4 h refeeding.

FIG. 6B. Increase in free Raptor levels does not affect mTORC1 or mTORC2 activity. Western blot from liver proteins from young or adult Ad-GFP or Ad-Raptor male mice, sacrificed after a 16 h fast followed by 4 h refeeding.

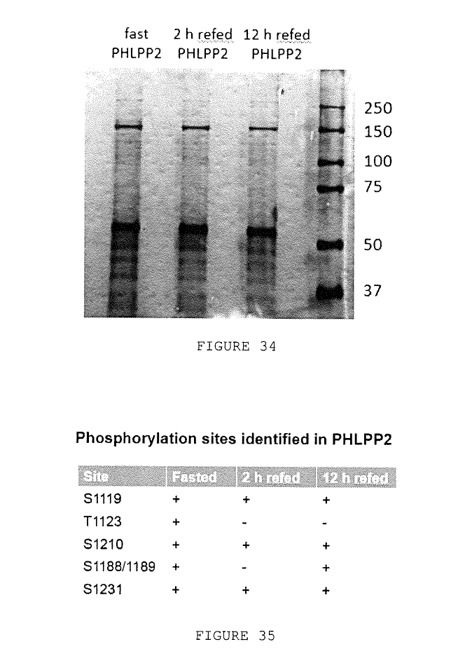

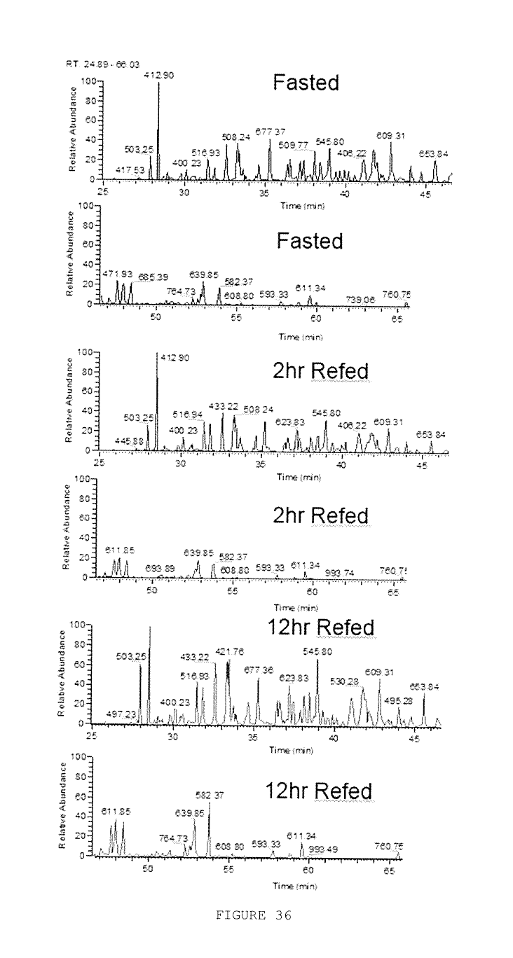

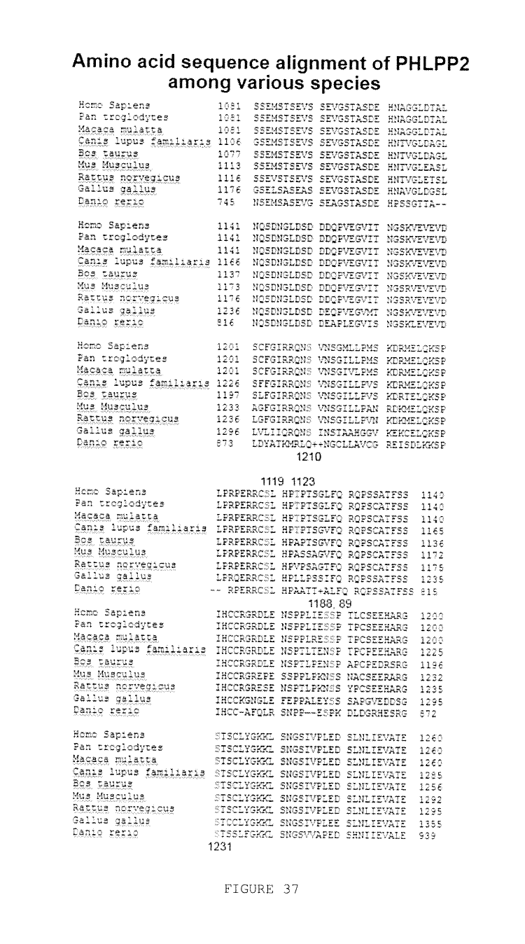

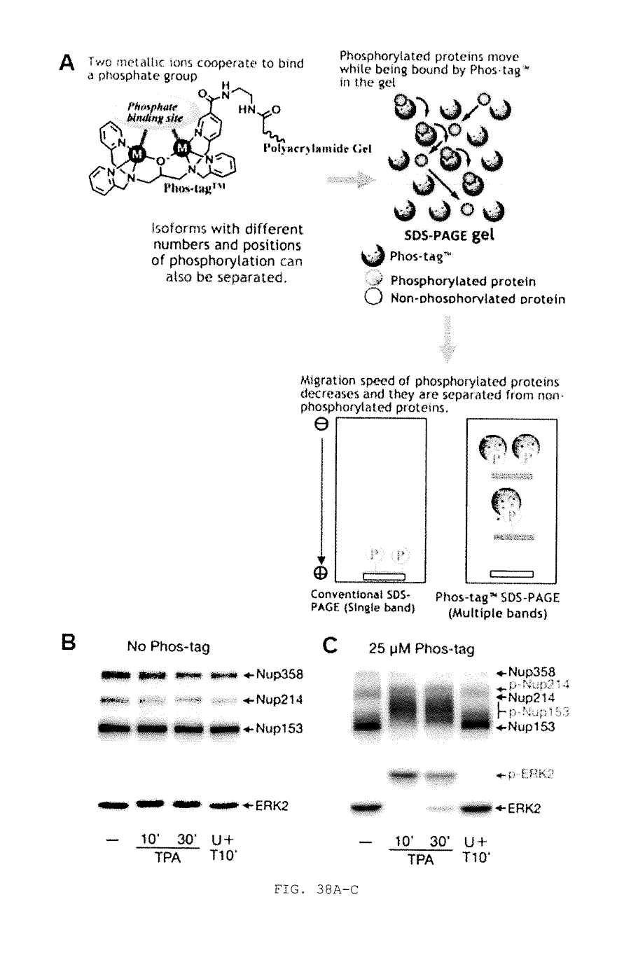

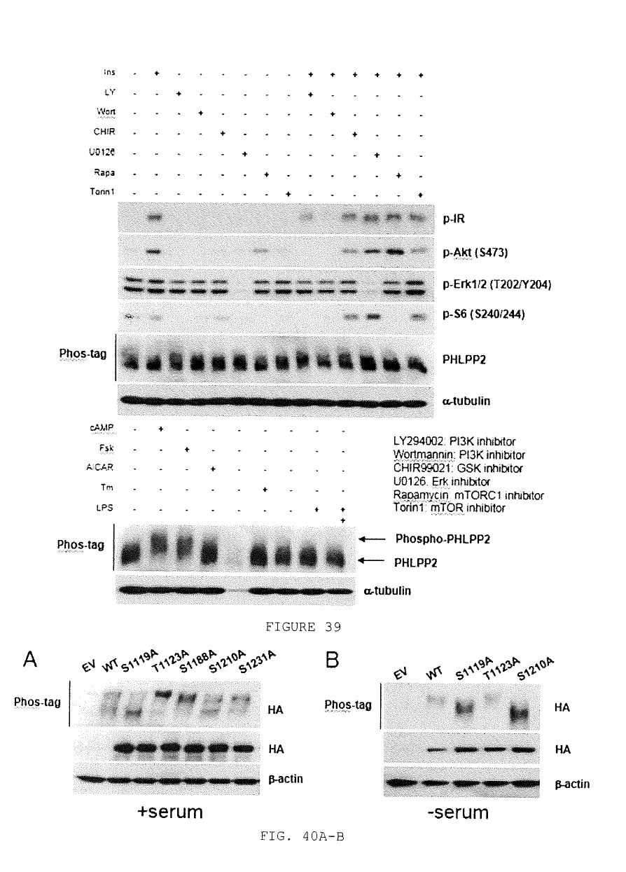

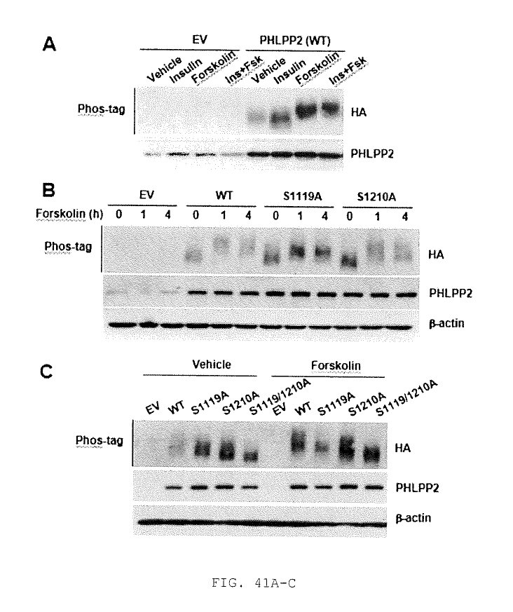

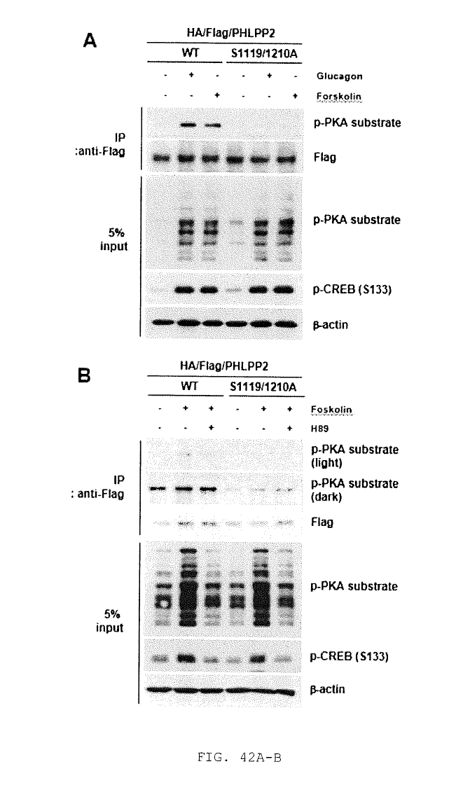

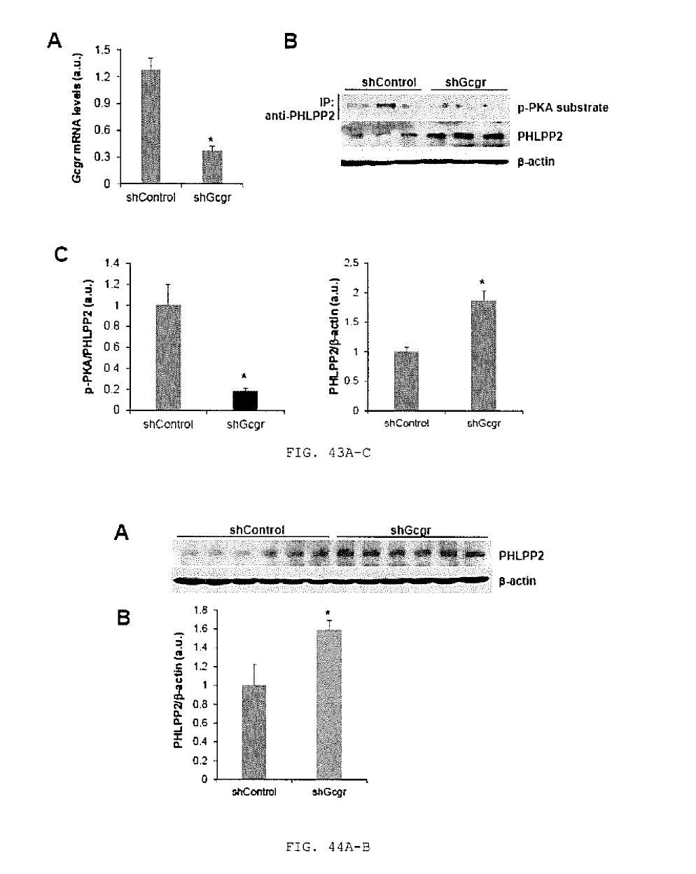

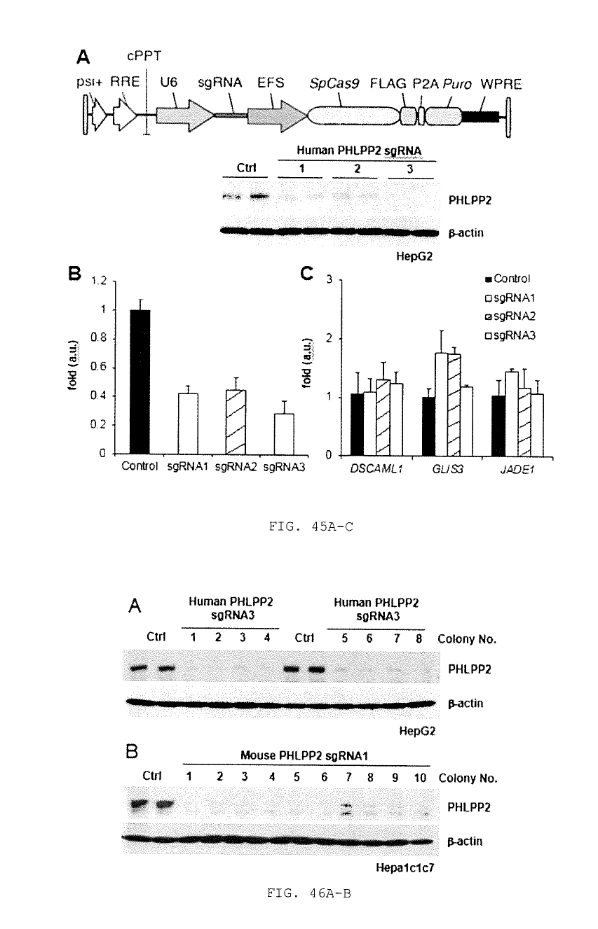

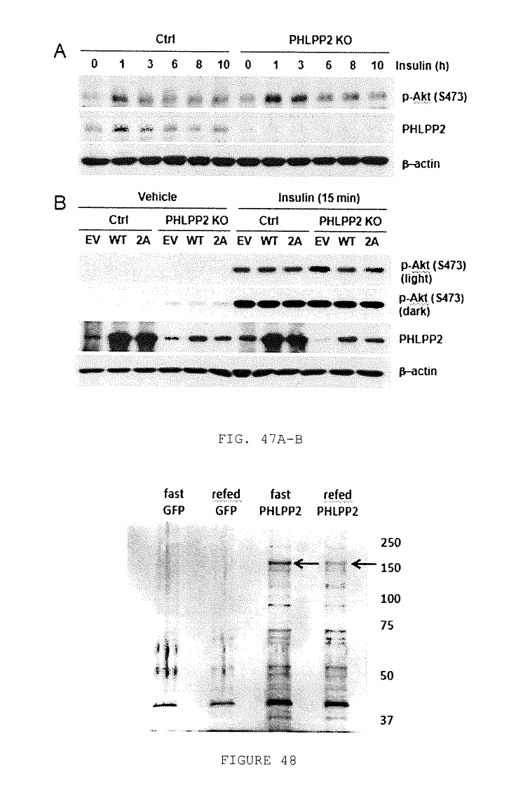

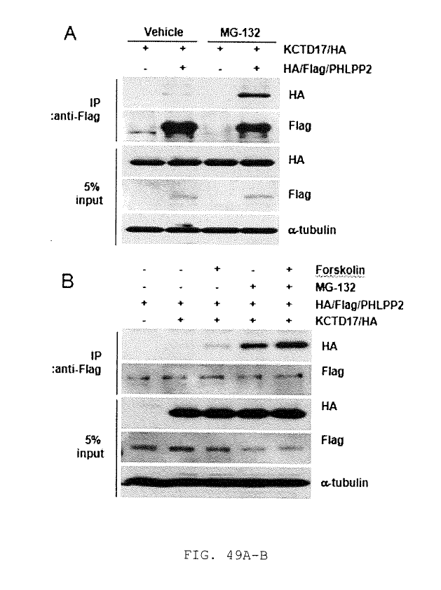

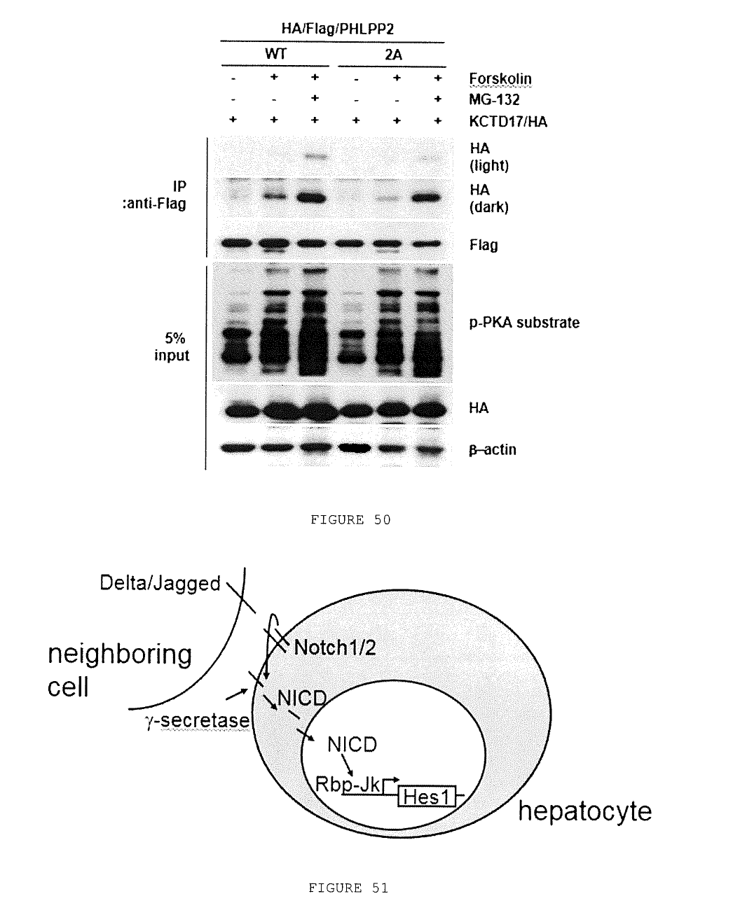

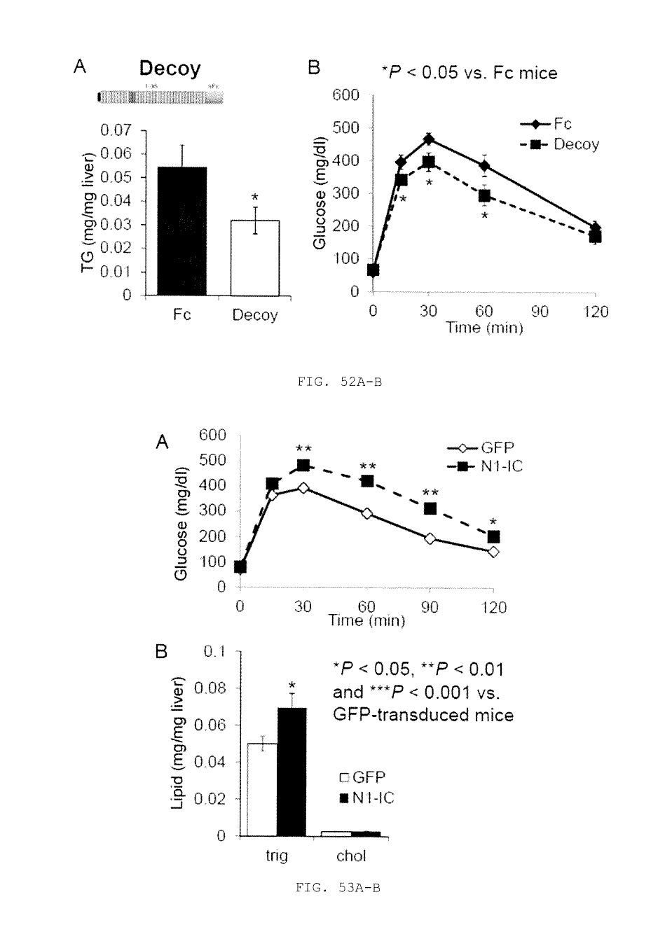

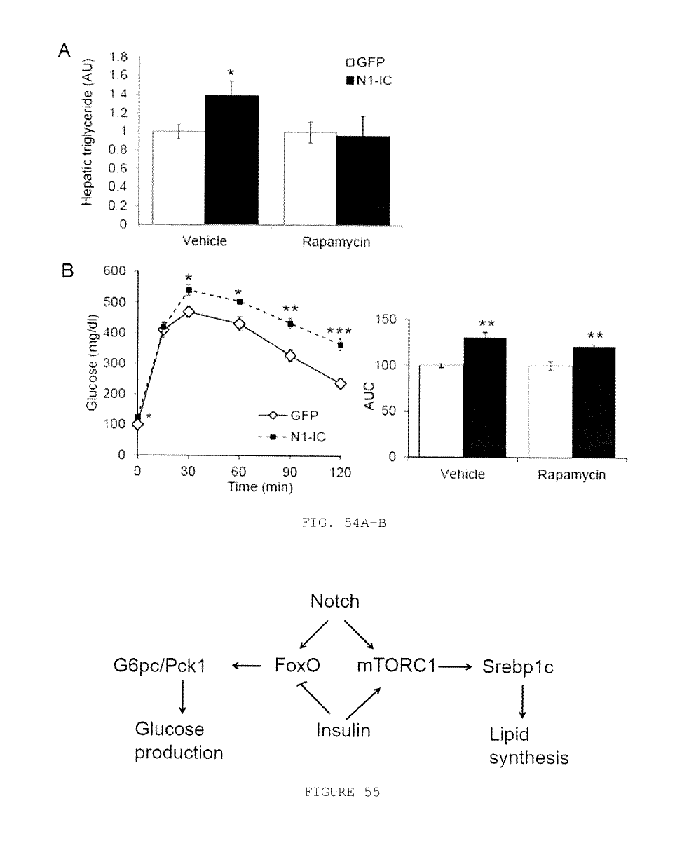

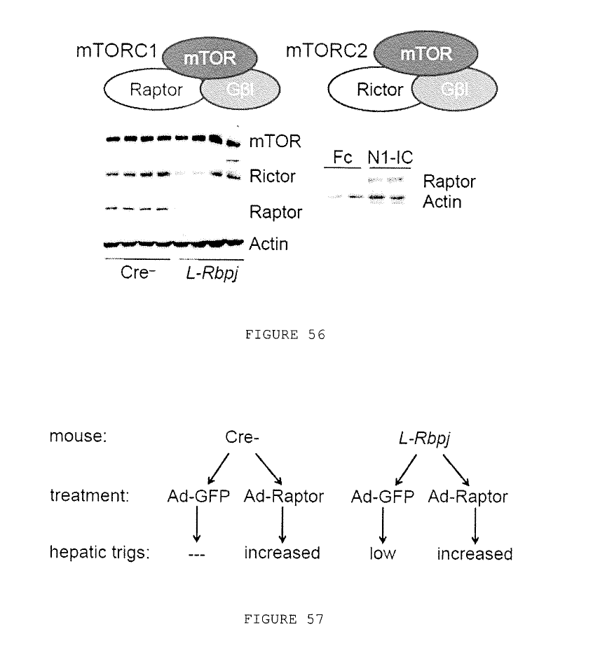

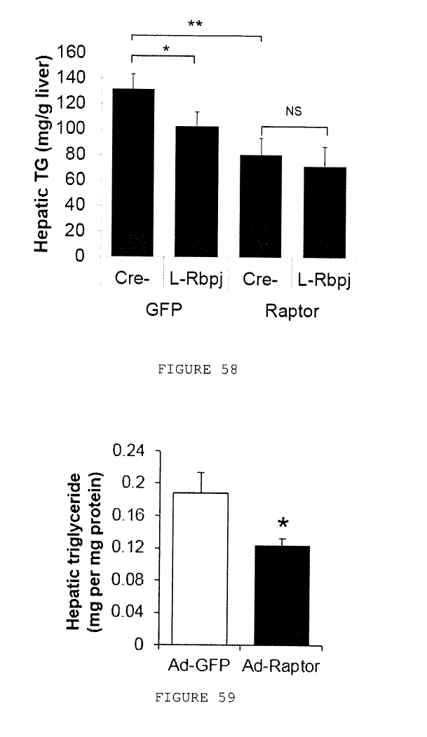

FIG. 5C. Total protein content in livers from aged (left), or young or adult Ad-GFP or Ad-Raptor male mice (right) mice, sacrificed after a 16 h fast followed by 4 h refeeding (n=7 or n=6 per each group).

FIG. 5D. Western blot from liver of young or adult Ad-GFP or Ad-Raptor male mice, sacrificed after a 16 h fast followed by 4 h refeeding.

FIG. 5E. Western blot from liver of young or adult Ad-GFP or Ad-Raptor male mice, sacrificed after a 16 h fast with or without 4 h refeeding.

FIG. 5F. Western blot from liver of young or adult Ad-GFP or Ad-Raptor male mice following IP with anti-mTOR antibody.

FIG. 7A. Free Raptor reduces aging/obesity-induced Akt hyperactivity. (A) Western blot and quantification from liver of young or adult Ad-GFP and Ad-Raptor mice sacrificed after a 16 h fast followed by 4 h refeeding. *P<0.05, **P<0.01 compared to the indicated control.

FIG. 7B. Plasma insulin (n=6/group) from liver of young or adult Ad-GFP and Ad-Raptor mice sacrificed after a 16 h fast followed by 4 h refeeding.

FIG. 7C. Western blot from liver of young or adult Ad-GFP and Ad-Raptor mice sacrificed after a 16 h fast followed by 4 h refeeding.

FIG. 7D. Akt activity on recombinant GSK3β peptide, normalized to immunoprecipitated Akt levels. *P<0.05, **P<0.01 compared to the indicated control.

FIG. 7E. Intraperitoneal glucose tolerance test (GTT) in young or adult Ad-GFP and Ad-Raptor mice (n=6 per each group).

FIG. 7F. Hepatic lipids from liver of adult Ad-GFP and Ad-Raptor mice co-transduced with control (GFP) or myrAkt adenovirus, sacrificed after a 16 h fast followed by 4 h refeeding (n=6 per each group).

FIG. 7G. Western blot from liver of adult Ad-GFP and Ad-Raptor mice co-transduced with control (GFP) or myrAkt adenovirus, sacrificed after a 16 h fast followed by 4 h refeeding (n=6 per each group). *P<0.05, **P<0.01 compared to the indicated control.

FIG. 8A. Free Raptor decreases Akt Ser473 phosphorylation, but does not affect gluconeogenic gene expression. Western blot from liver of young or adult Ad-GFP or Ad-Raptor male mice, sacrificed after a 16 h fast with or without 4 h refeeding.

FIG. 8B. Western blot from eWAT of young or adult Ad-GFP or Ad-Raptor male mice sacrificed after a 16 h fast followed by 4 h refeeding.

FIG. 8C. Western blot from liver of adult Ad-GFP or Ad-Raptor male mice sacrificed after a 16 h fast followed by 4 h refeeding.

FIG. 8D. Western blot from liver of adult Ad-GFP or Ad-Raptor male mice sacrificed after a 16 h fast followed by 4 h refeeding.

FIG. 8E. Western blot from primary hepatocytes transduced with Ad-GFP or Ad-Raptor, then incubated with 10 nM insulin for 30 min.

FIG. 8F. Blood glucose in young or adult Ad-GFP or Ad-Raptor male mice sacrificed after a 16 h fast with or without 4 h refeeding.(n=6 per each group).

FIG. 8G. Gluconeogenic gene expression in young or adult Ad-GFP or Ad-Raptor male mice sacrificed after a 16 h fast with or without 4 h refeeding. (n=6 per each group).

FIG. 9A. Body weight of adult Ad-GFP or Ad-Raptor mice co-transduced with control (GFP) or constitutively-active, myristoylated Akt (myrAkt) adenovirus sacrificed after a 16 h fast followed by 4 h refeeding (n=6 per each group).

FIG. 9B. eWAT weight of adult Ad-GFP or Ad-Raptor mice co-transduced with control (GFP) or constitutively-active, myristoylated Akt (myrAkt) adenovirus sacrificed after a 16 h fast followed by 4 h refeeding (n=6 per each group).

FIG. 9C. Blood glucose levels in adult Ad-GFP or Ad-Raptor mice co-transduced with GFP or myrAkt adenovirus either fasted for 16 h or fasted for 16 h and refed for 4 h.

FIG. 9D. Rescue of Akt activity increases hepatic TG in Ad-Raptor mice. Hepatic protein content in livers from adult Ad-GFP or Ad-Raptor mice co-transduced with GFP or myrAkt adenovirus sacrificed after a 16 h fast followed by 4 h refeeding (n=6 per each group).

FIG. 10A. Western blot of PHLPP isoforms from Hepa1c1c7 cells or liver of adult mice.

FIG. 10B. PHLPP2 knockdown increases Akt S473 phosphorylation and activity. Western blot from primary hepatocytes transduced with Ad-shControl, Ad-shPhlpp1, or Ad-shPhlpp2 adenoviruses.

FIG. 10C. Glucose tolerance test (GTT) and area under the curve (AUC) from GTT in 16 h fasted adult Ad-shControl, AdshPhlpp1, or Ad-shPhlpp2 mice (n=6 per each group). *P<0.05 and **P<0.01 as compared to the indicated control.

FIG. 10D. Plasma insulin levels in adult Ad-shControl, AdshPhlpp1, or Ad-shPhlpp2 after a 16 h fast with or without 4 h refeeding. *P<0.05 and **P<0.01 as compared to the indicated control.

FIG. 10E. Liver weight in livers of adult Ad-shControl, Ad-shPhlpp1, or Ad-shPhlpp2 mice sacrificed after a 16 h fast followed by 4 h refeeding (n=6 per each group). *P<0.05 and **P<0.01 as compared to the indicated control.

FIG. 10F. Oil-Red-O or H&E staining in livers of adult Ad-shControl, Ad-shPhlpp1, or Ad-shPhlpp2 mice sacrificed after a 16 h fast followed by 4 h refeeding (n=6 per each group).

FIG. 11A. Western blot from liver of adult Ad-GFP and Ad-Raptor mice sacrificed after a 16 h fast followed by 4 h refeeding.

FIG. 11B. Western blot in livers of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6 per each group).

FIG. 11C. Reduced liver TG in Ad-Raptor mice is PHLPP2-dependant. Hepatic lipids in livers of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6 per each group). *P<0.05, **P<0.01 compared to the indicated control.

FIG. 11D. Plasma lipids in livers of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6 per each group). *P<0.05, **P<0.01 compared to the indicated control.

FIG. 11E. Gene expression in livers of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6 per each group). *P<0.05, **P<0.01 compared to the indicated control.

FIGS. 12A-H. Free Raptor, but not mTORC1 activity, increases PHLPP2 protein to reduce liver TG. Body weight (A), eWAT weight (B), hepatic protein content (C), β-hydroxybutyrate levels (D) and NEFA levels (E) in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group). Western blot from (F) primary hepatocytes treated with vehicle (Veh) or Rapamycin (20 nM), or (G) Tsc+/+ and Tsc2−/− MEFs. (H) PHLPP1 and 2 gene expression in Ad-GFP or Ad-Raptor-transduced primary hepatocytes.

FIG. 12A. Body weight in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group).

FIG. 12B. eWAT weight in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group).

FIG. 12C. Hepatic protein content in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group).

FIG. 12D. β-hydroxybutyrate levels in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group).

FIG. 12E. NEFA levels in adult Ad-GFP or Ad-Raptor mice co-transduced with Ad-shControl, Ad-shPhlpp1, or Ad-shPhlpp2, sacrificed after a 16 h fast followed by 4 h refeeding (n=6 per each group).

FIG. 12F. Western blot from primary hepatocytes treated with vehicle (Veh) or Rapamycin (20 nM).

FIG. 12G. Western blot from Tsc+/+ and Tsc2−/− MEFs.

FIG. 12H. PHLPP1 and 2 gene expression in Ad-GFP or Ad-Raptor-transduced primary hepatocytes.

FIG. 13A. Raptor overexpression blocks PHLPP2 proteosomal degradation. Western blot from Hepa1c1c7 cells treated with Rapamycin (20 nM) or Torin1 (250 nM).

FIG. 13B. Raptor overexpression blocks PHLPP2 proteosomal degradation. Western blot from primary hepatocytes transduced with GFP or Raptor, treated with Rapamycin (20 nM) or Torin1 (250 nM).

FIG. 13C. Western blot from HFD-fed Raptor fl/fl liver transduced with AAV8-TBG-GFP or AAV8-TBG-Cre.

FIG. 13D. Hepatic gene expression from young or adult mice Ad-GFP and Ad-Raptor sacrificed after a 16 h fast followed by 4 h refeeding.

FIG. 13E. Western blot of primary hepatocytes co-transduced with PHLPP1 or PHLPP2 and Raptor (or GFP control) adenovirus.

FIG. 13F. Western blot from cycloheximide (CHX, 50 mg/ml)-treated Hepa1c1c7 cells, with quantification of PHLPP2 normalized to β-Actin relative to t=0 h.

FIG. 13G. Western blot of Raptor (or GFP control)-transduced Hepa1c1c7 cells following immunoprecipitation with anti-PHLPP2, with or without MG-132.

FIG. 13H. Western blot of Raptor (or GFP control)-transduced Hepa1c1c7 cells following immunoprecipitation with anti-PHLPP2, with or without MG-132.

FIG. 13I. Western blot from liver of young or adult Ad-GFP and Ad-Raptor mice following immunoprecipitation with anti-Akt, with quantitation of PHLPP1 or PHLPP2 normalized to immunoprecipitated Akt levels. *P<0.05 compared to the indicated control.

FIG. 14A. PHLPP2 overexpression does not affect body weight or glucose homeostasis. Body weight in adult, HFD-fed Ad-GFP or Ad-PHLPP2 male mice, sacrificed after a 16 h fast followed by 4 h refeeding (n=6 or 7 per each group).

FIG. 14B. eWAT weight in adult, HFD-fed Ad-GFP or Ad-PHLPP2 male mice, sacrificed after a 16 h fast followed by 4 h refeeding (n=6 or 7 per each group).

FIG. 14C. Hepatic protein content in adult, HED-fed Ad-GFP or Ad-PHLPP2 male mice, sacrificed after a 16 h fast followed by 4 h refeeding (n=6 or 7 per each group).

FIG. 14D. Hepatic cholesterol in adult, HFD-fed Ad-GFP or Ad-PHLPP2 male mice, sacrificed after a 16 h fast followed by 4 h refeeding (n=6 or 7 per each group).

FIG. 14E. PHLPP2 overexpression does not affect body weight or glucose homeostasis. Blood glucose in adult, HFD-fed Ad-GFP or Ad-PHLPP2 male mice, sacrificed after a 16 h fast followed by 4 h refeeding (n=6 or 7 per each group).

FIG. 14F. PHLPP2 overexpression does not affect body weight or glucose homeostasis. Insulin levels in adult, HFD-fed Ad-GFP or Ad-PHLPP2 male mice, sacrificed after a 16 h fast followed by 4 h refeeding (n=6 or 7 per each group).

FIG. 14F. PHLPP2 overexpression does not affect body weight or glucose homeostasis. Body weight (A), eWAT weight (B), hepatic protein content (C), hepatic cholesterol (D), blood glucose (E) and insulin levels (F) in adult, HFD-fed Ad-GFP or Ad-PHLPP2 male mice, sacrificed after a 16 h fast followed by 4 h refeeding (n=6 or 7 per each group).

FIG. 15A. Hepatic gene expression from liver of young and adult mice, with quantitation normalized to β-actin (n=6 per each group).

FIG. 15B. Western blot from liver of young and adult mice, with quantitation normalized to β-actin (n=6 per each group).

FIG. 15C. Western blot from liver of adult chow- and HFD-fed mice.

FIG. 15D. Western blot from liver of lean and ob/ob mice.

FIG. 15E. Western blot from liver of adult, HFD-fed Ad-GFP or Ad-PHLPP2 mice sacrificed after a 16 h fast followed by a 4 h refeeding.

FIG. 15F. Rescue of aging/obesity-reduced PHLPP2 levels prevents hepatic steatosis. Liver weight of adult, HFD-fed Ad-GFP or Ad-PHLPP2 mice sacrificed after a 16 h fast followed by a 4 h refeeding (n=6 or 7 per each group). *P<0.05, **P<0.01 compared to the indicated control.

FIG. 15G. Lipid content of adult, HFD-fed Ad-GFP or Ad-PHLPP2 mice sacrificed after a 16 h fast followed by a 4 h refeeding (n=6 or 7 per each group).

FIG. 15H. Lipogenic genes of adult, HFD-fed Ad-GFP or Ad-PHLPP2 mice sacrificed after a 16 h fast followed by a 4 h refeeding (n=6 or 7 per each group). *P<0.05, **P<0.01 compared to the indicated control.

FIG. 15I. Plasma lipids in adult, HFD-fed Ad-GFP or Ad-PHLPP2 mice (n=6 or 7 per each group). *P<0.05, **P<0.01 compared to the indicated control.

FIG. 15J. GTT in adult, HFD-fed Ad-GFP or Ad-PHLPP2 mice (n=6 or 7 per each group).

FIG. 16. A proposed model of Raptor-mediated PHLPP2 regulation in hepatic steatosis.

FIG. 17A. “Free” Raptor levels decline in aged and obese liver. Western blots from livers of young (8-week-old), adult (24-week-old), and ob/ob (8-week-old) male mice, following size-exclusion chromatography.

FIG. 17B. “Free” Raptor levels decline in aged and obese liver. Quantification of signal per fraction (FIG. 17A), normalized to total protein.

FIG. 17C. Western blots from livers of young, adult and ob/ob male mice, cross-linked with DSS.

FIG. 17D. Quantification of free Raptor signal, as a percentage of control (DMSO-treated liver lysate).

FIG. 17E. Western blot from hepatocytes deprived of amino acids or treated with rapamycin, prior to DSS crosslinking. *P<0.05 as compared to the indicated control.

FIG. 17F. Western blot from hepatocytes deprived of amino acids or treated with insulin, prior to DSS crosslinking. *P<0.05 as compared to the indicated control.

FIG. 18A. Western blots from livers of adult Ad-GFP and Ad-Raptor male mice, following size-exclusion chromatography. Fractions 24 and 25 correspond to mTORC1-associated (^{˜}800 kDa) Raptor, while 34 and 35 to free (^{˜}150 kDa) Raptor.

FIG. 18B. Rescue of free Raptor prevents aging- and obesity-dependent hepatic steatosis. Hepatic TG in young or adult (n=6/group) male mice. *P<0.05, **P<0.01 as compared to the indicated control.

FIG. 18C. Rescue of free Raptor prevents aging- and obesity-dependent hepatic steatosis. Hepatic TG in aged (10- to 12-month-old) (n=7/group) male mice. *P<0.05, **P<0.01 as compared to the indicated control.

FIG. 18D. Rescue of free Raptor prevents aging- and obesity-dependent hepatic steatosis. Hepatic TG in diet-induced obese (DIO, n=5/group) male mice. *P<0.05, **P<0.01 as compared to the indicated control.

FIG. 18E. Rescue of free Raptor prevents aging- and obesity-dependent hepatic steatosis. Plasma TG in young or adult, Ad-GFP and Ad-Raptor mice, sacrificed after a 16 h fast followed by 4 h refeeding (n=6/group). *P<0.05, **P<0.01 as compared to the indicated control.

FIG. 18F. Rescue of free Raptor prevents aging- and obesity-dependent hepatic steatosis. Hepatic fatty acid (FA) synthesis in young or adult, Ad-GFP and Ad-Raptor mice, sacrificed after a 16 h fast followed by 4 h refeeding (n=6/group). *P<0.05, **P<0.01 as compared to the indicated control.

FIG. 18G. Rescue of free Raptor prevents aging- and obesity-dependent hepatic steatosis. Lipogenic gene expression in young or adult, Ad-GFP and Ad-Raptor mice, sacrificed after a 16 h fast followed by 4 h refeeding (n=6/group). *P<0.05, **P<0.01 as compared to the indicated control.

FIG. 19A. Western blots from livers of young or adult Ad-GFP and Ad-Raptor mice sacrificed after a 16 h fast followed by 4 h refeeding.

FIG. 19B. Western blots from livers of young or adult Ad-GFP and Ad-Raptor mice sacrificed after a 16 h fast followed by 4 h refeeding.

FIG. 19C. Free Raptor prevents prolonged Akt activity by stabilizing PHLPP2. Akt activity on recombinant GSK3β peptide, and normalization to immunoprecipitated Akt levels.

FIG. 19D. Free Raptor prevents prolonged Akt activity by stabilizing PHLPP2. Western blots from liver of adult Ad-GFP and Ad-Raptor mice sacrificed after a 16 h fast followed by 4 h refeeding, and normalization to β-actin.

FIG. 19E. Western blots from Hepa1c1c7 cells treated with Rapamycin (20 nM) or Torin1 (250 nM).

FIG. 19F. Western blots from primary hepatocytes transduced with Ad-shControl or Ad-shmTOR.

FIG. 19G. Western blot of primary hepatocytes transduced with GFP or Raptor adenovirus, treated for 0-8 h with cycloheximide (CHX, 50 mg/ml), and PHLPP2 quantification, normalized to β-actin and expressed as % baseline (t=0 h).

FIG. 19H. Western blots of Raptor (or GFP control)-transduced and/or HA/Ub-transfected primary hepatocytes following immunoprecipitation with anti-PHLPP2, with or without MG-132. Blots are representative of three independent experiments.

FIG. 19I. Western blots of Raptor (or GFP control)-transduced and/or HA/Ub-transfected primary hepatocytes following immunoprecipitation with anti-PHLPP2, with or without MG-132. Blots are representative of three independent experiments.

FIG. 20A. Liver mRNA expression from young and adult mice (n=6/group).

FIG. 20B. Western blots from livers of young and adult mice, and normalization to β-actin.

FIG. 20C. Western blots from livers of chow- and HFD-fed mice, and normalization to β-actin.

FIG. 20D. Western blots from livers of lean and ob/ob mice, and normalization to β-actin.

FIG. 20E. Rescue of lower PHLPP2 in aging or obesity reduces lipogenesis. Liver Western blots in adult, HFD-fed Ad-GFP or Ad-PHLPP2 mice (n=6-7/group).

FIG. 20F. Rescue of lower PHLPP2 in aging or obesity reduces lipogenesis. Hepatic TG in adult, HFD-fed Ad-GFP or Ad-PHLPP2 mice (n=6-7/group).

FIG. 20G. Rescue of lower PHLPP2 in aging or obesity reduces lipogenesis. Lipogenic gene expression in adult, HFD-fed Ad-GFP or Ad-PHLPP2 mice (n=6-7/group). *P<0.05, **P<0.01 as compared to the indicated control.

FIG. 20H. Rescue of lower PHLPP2 in aging or obesity reduces lipogenesis. Plasma TG in adult, HFD-fed Ad-GFP or Ad-PHLPP2 mice (n=6-7/group). *P<0.05, **P<0.01 as compared to the indicated control.

FIG. 21A. Reduced liver TG by free Raptor is PHLPP2-dependent. Western blots from liver of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6/group).

FIG. 21B. Reduced liver TG by free Raptor is PHLPP2-dependent. Hepatic TG of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6/group). *P<0.05, **P<0.01 as compared to the indicated control.

FIG. 21C. Reduced liver TG by free Raptor is PHLPP2-dependent. Plasma TG of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6/group). *P<0.05, **P<0.01 as compared to the indicated control.

FIG. 21D. Reduced liver TG by free Raptor is PHLPP2-dependent. Liver lipogenic gene expression of adult Ad-GFP and Ad-Raptor mice co-transduced with control (Ad-shControl), Ad-shPHLPP1 or Ad-shPHLPP2 adenoviruses, sacrificed after a 16 h fast followed by 4 h refeeding (n=6/group). *P<0.05, **P<0.01 as compared to the indicated control.

FIG. 21E. Hepatic TG of adult Ad-GFP and Ad-Raptor mice co-transduced with control (GFP) or myrAkt adenovirus, sacrificed after a 16 h fast followed by 4 h refeeding (n=6/group). *P<0.05, **P<0.01 as compared to the indicated control.

FIG. 21F. GTT in adult, HFD-fed Ad-GFP or Ad-PHLPP2 mice (n=6-7/group).

FIG. 22. A proposed model of free Raptor-mediated regulation of hepatic lipogenesis.

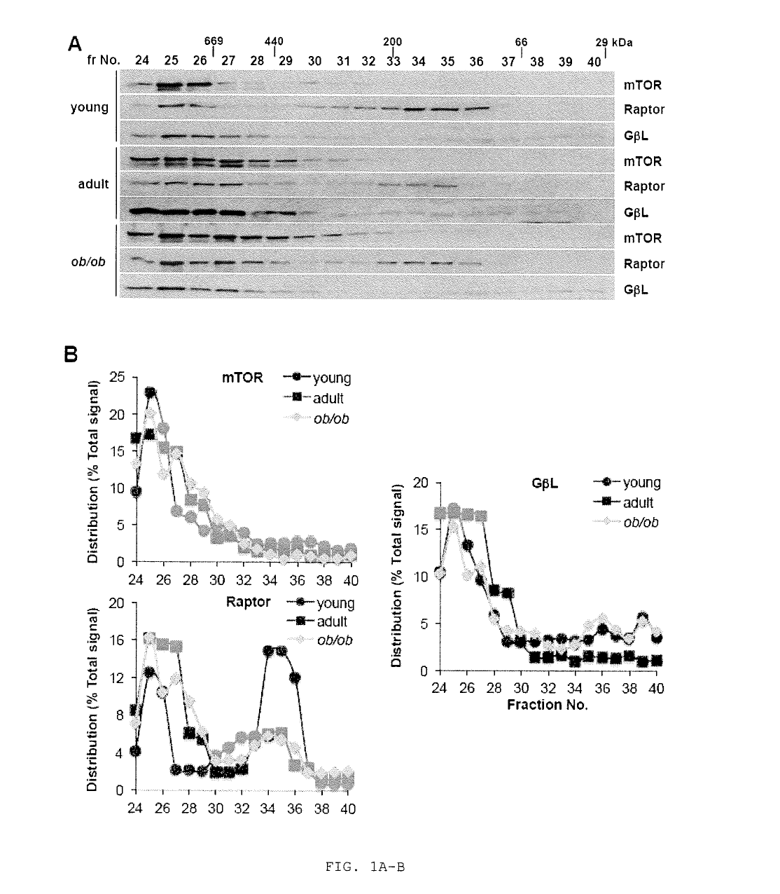

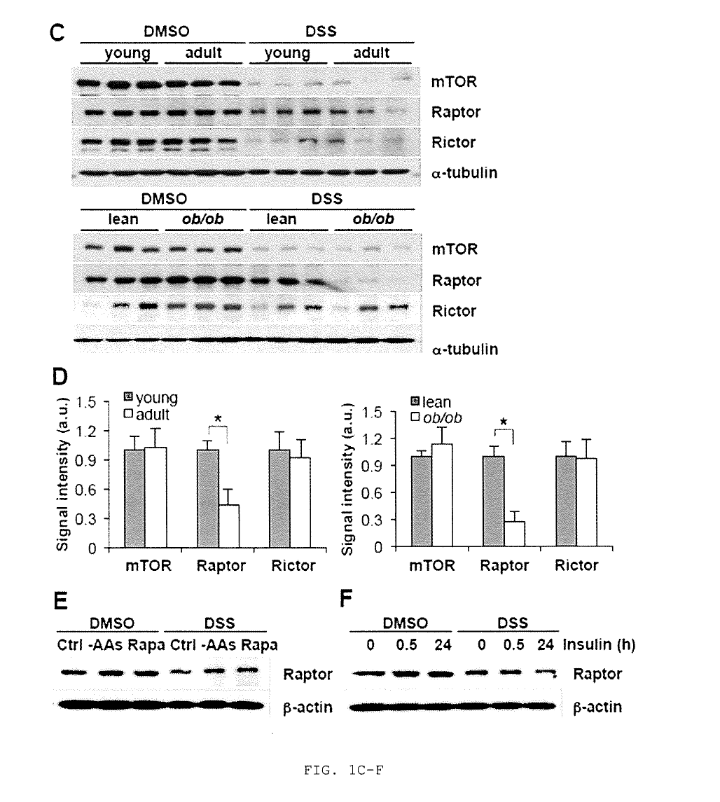

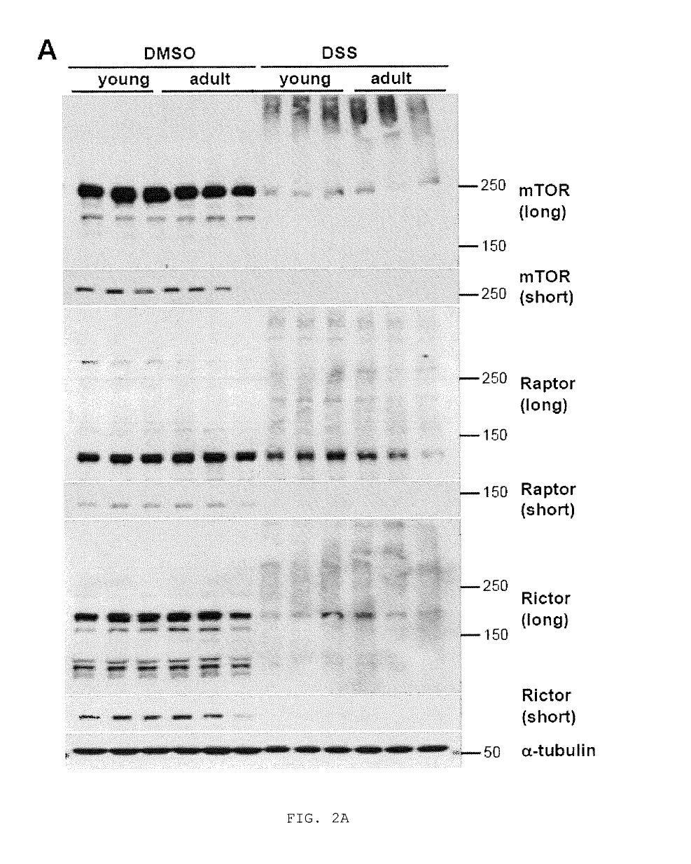

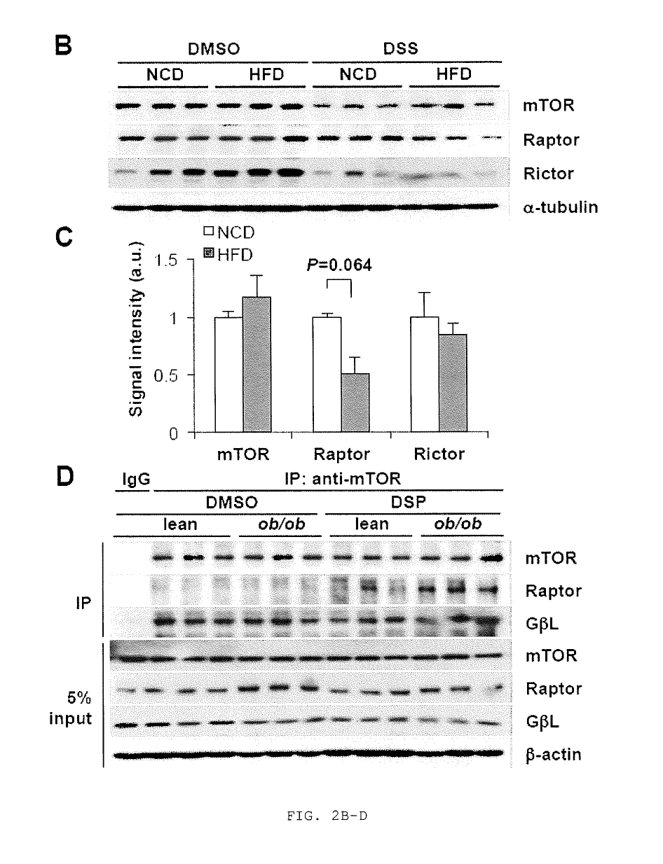

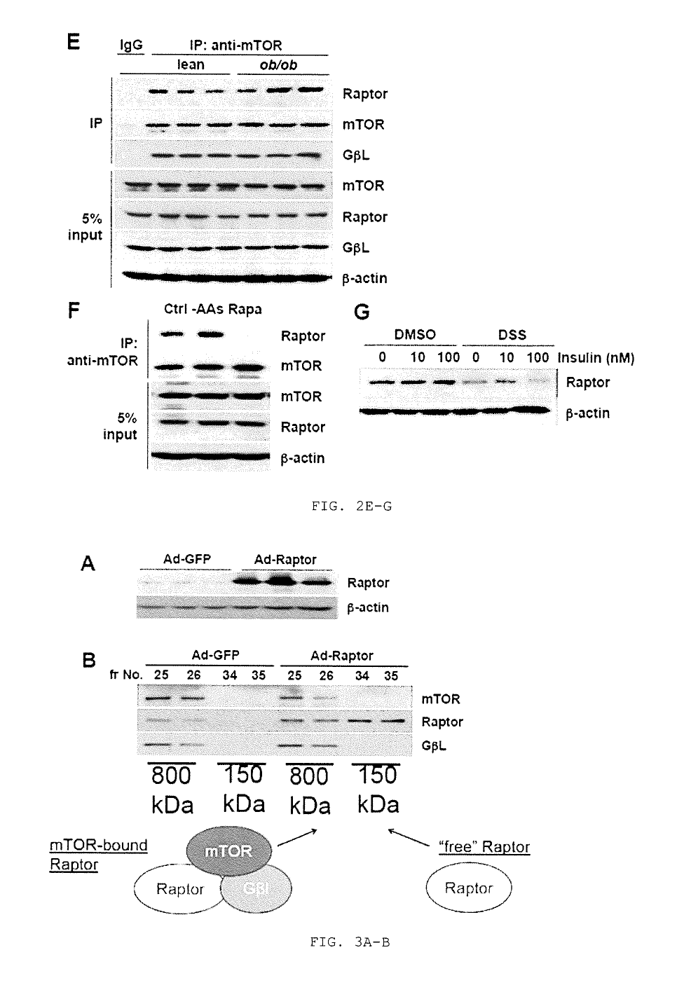

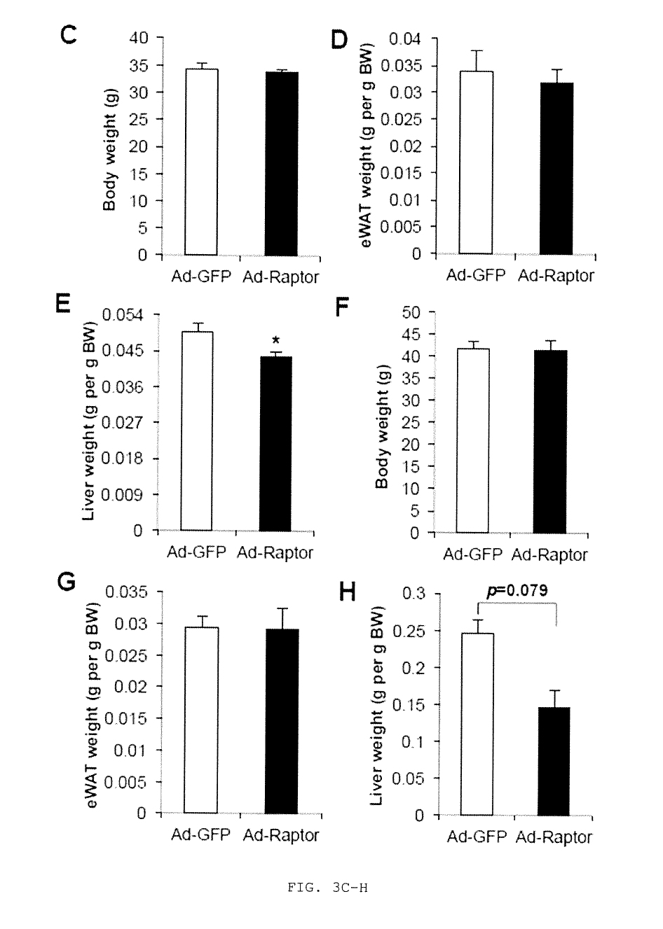

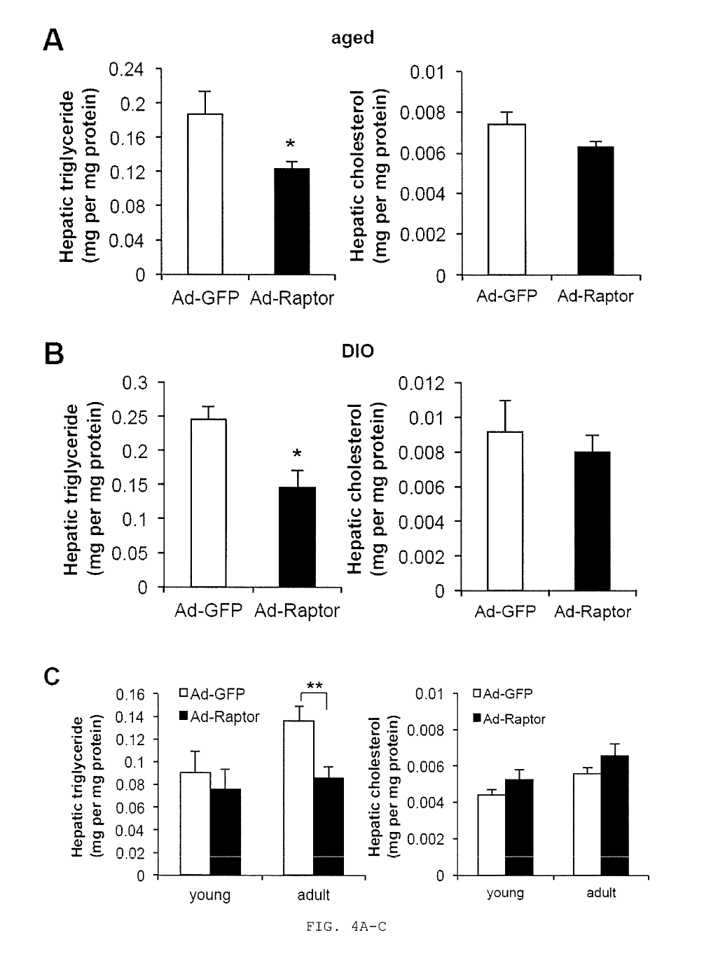

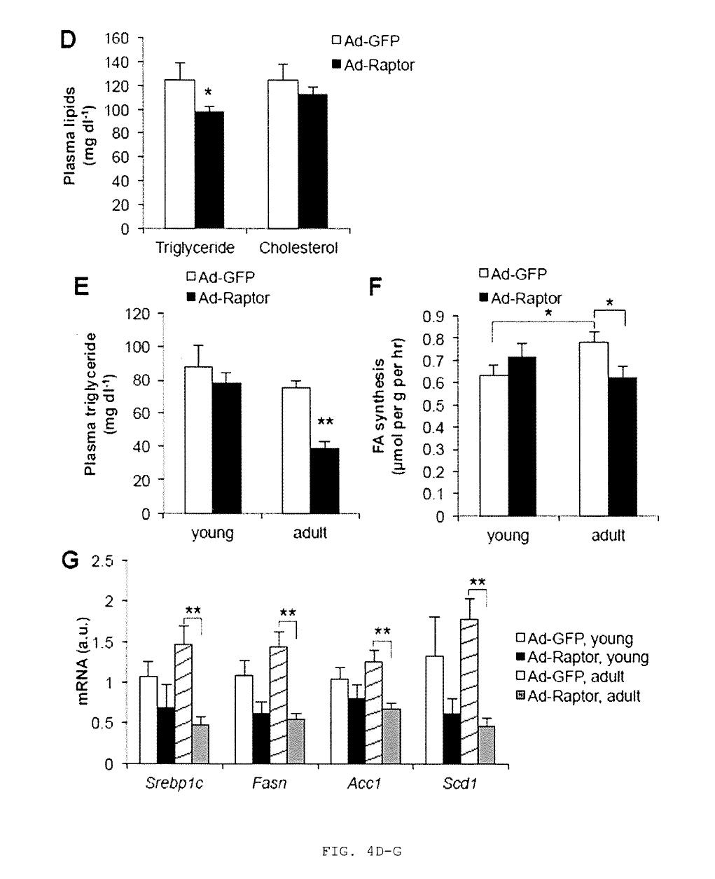

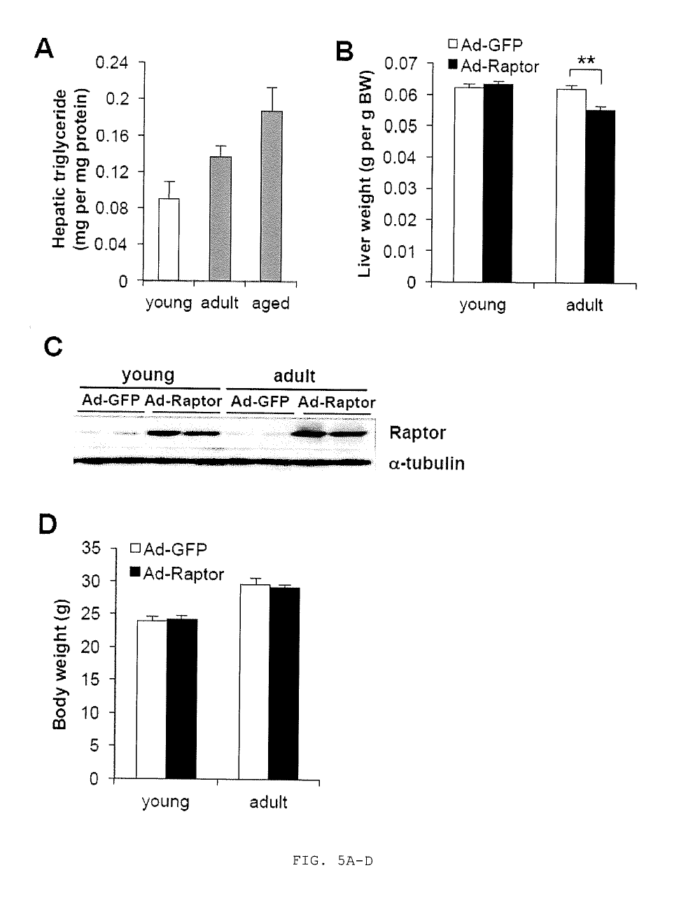

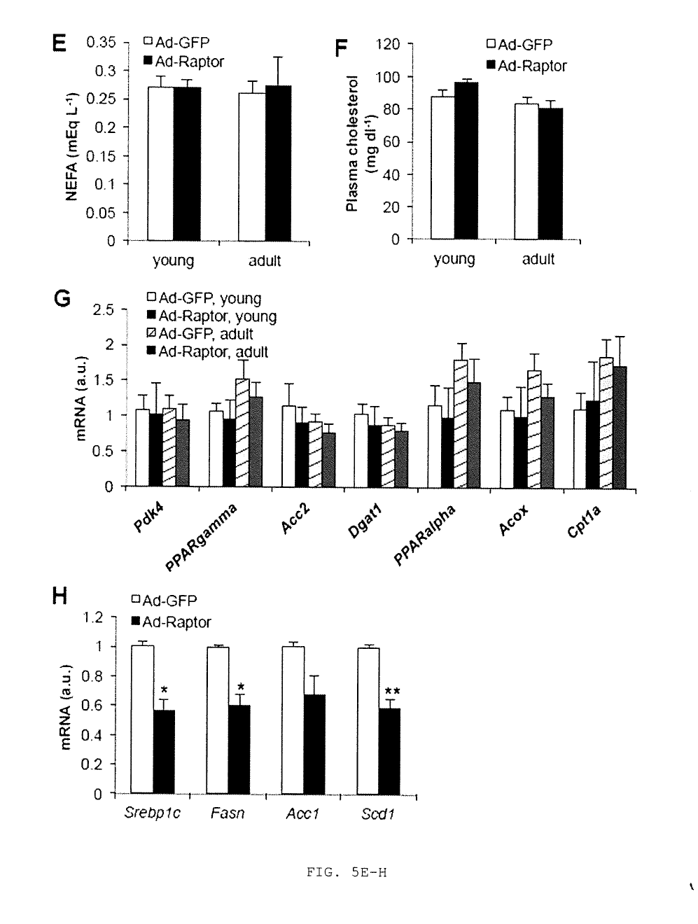

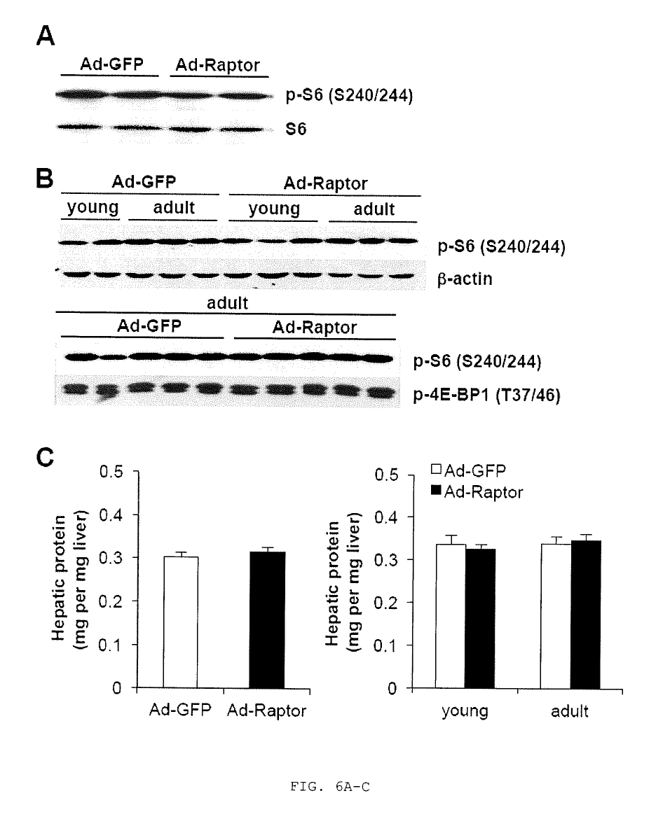

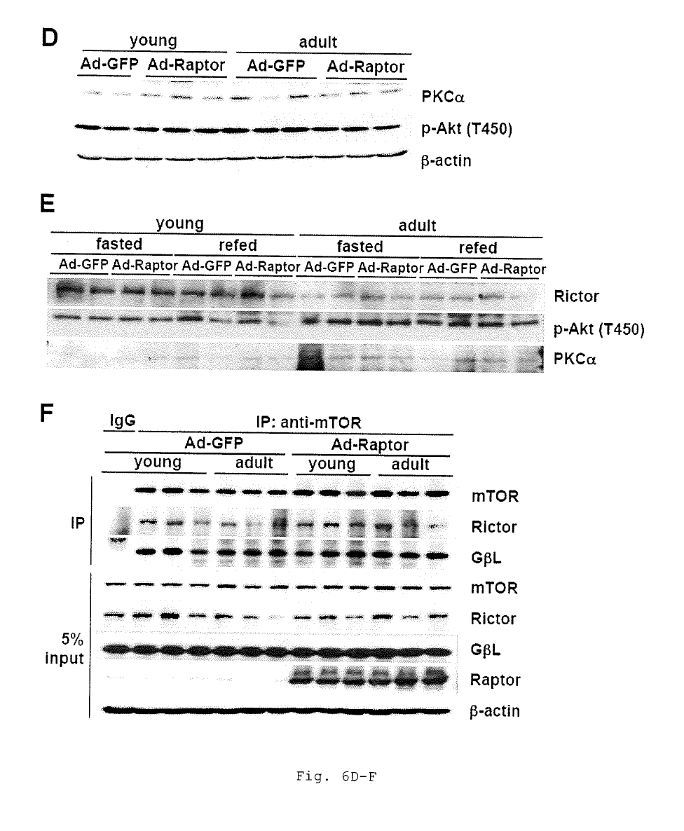

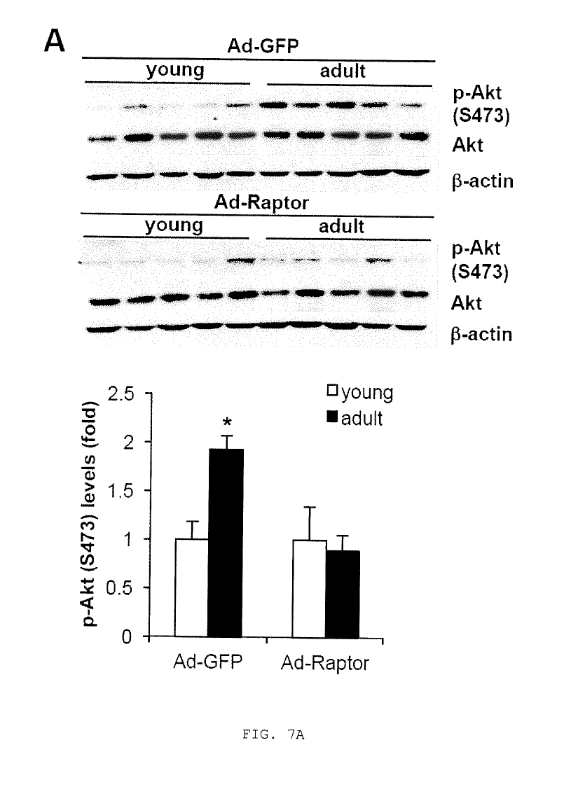

FIG. 23A. mTORC1-independent Raptor is reduced in insulin-resistant liver. Western blots from livers of young (8-week-old) or adult (24-week-old) male mice, showing the whole blots corresponding to FIG. 1C.

FIG. 23B. mTORC1-independent Raptor is reduced in insulin-resistant liver. Western blots from livers of normal chow diet (NCD) or HFD-fed male mice, crosslinked with DSS.

FIG. 23C. mTORC1-independent Raptor is reduced in insulin-resistant liver. Quantitation of signal as a percentage of control (DMSO-treated liver lysate).

FIG. 23D. Western blots from livers of lean or ob/ob mice (8-week-old) following immunoprecipitation (IP) with anti-mTOR, with or without prior crosslinking with DSP.

FIG. 23E. Western blots from livers of lean or ob/ob mice (8-week-old) following immunoprecipitation (IP) with anti-mTOR, in the presence of CHAPS to sustain mTOR-Raptor interaction.

FIG. 23F. Western blot from primary hepatocytes deprived of amino acids (-AA) or treated with rapamycin (Rapa) for 1 h prior to IP with anti-mTOR.

FIG. 23G. Western blot from Hepa1c1c7 cells, treated with varying concentrations of insulin for 24 hours, prior to DSS crosslinking. Blots are representative of three independent experiments.

FIG. 24A. Western blot from livers of young or adult Ad-GFP and Ad-Raptor mice sacrificed after a 16 h fast followed by 4 h refeeding (n=6 per each group).

FIG. 24B. Free Raptor reduces liver weight and TG content in older or obese mice. Hepatic TG content in young (8-week-old), adult (24-week-old) or aged (10- to 12-month-old) male mice sacrificed after a 16 h fast followed by 4 h refeeding (n=6/group).

FIG. 24C. Free Raptor reduces liver weight and TG content in older or obese mice. Liver weight in aged (10- to 12-month-old, n=7/group). *P<0.05 as compared to the indicated control.

FIG. 24D. Free Raptor reduces liver weight and TG content in older or obese mice. Liver weight in DIO (n=5/group) Ad-GFP and Ad-Raptor mice.

FIG. 25A. Free Raptor reduces lipogenesis without affecting body weight, adiposity or fatty acid oxidation. Body weight in young or adult Ad-GFP and Ad-Raptor mice, sacrificed after a 16 h fast followed by 4 h refeeding (n=6/group).

FIG. 25B. Free Raptor reduces lipogenesis without affecting body weight, adiposity or fatty acid oxidation. Epididymal fat pad (eWAT) weight in young or adult Ad-GFP and Ad-Raptor mice, sacrificed after a 16 h fast followed by 4 h refeeding (n=6/group).

FIG. 25C. Free Raptor reduces lipogenesis without affecting body weight, adiposity or fatty acid oxidation. Non-esterified fatty acid (NEFA) levels in young or adult Ad-GFP and Ad-Raptor mice, sacrificed after a 16 h fast followed by 4 h refeeding (n=6/group).

FIG. 25D. Free Raptor reduces lipogenesis without affecting body weight, adiposity or fatty acid oxidation. Liver mRNA expression in young or adult Ad-GFP and Ad-Raptor mice, sacrificed after a 16 h fast followed by 4 h refeeding (n=6/group).

FIG. 25E. Free Raptor reduces lipogenesis without affecting body weight, adiposity or fatty acid oxidation. Plasma β-hydroxybutyrate levels in young or adult Ad-GFP and Ad-Raptor mice, sacrificed after a 16 h fast followed by 4 h refeeding (n=6/group).

FIG. 25F. Free Raptor reduces lipogenesis without affecting body weight, adiposity or fatty acid oxidation. Srebp1c-dependent lipogenic gene expression in primary hepatocytes transduced with Ad-GFP or Ad-Raptor (n=4 biologic replicates). *P<0.05 and **P<0.01 as compared to the indicated control.

FIG. 26A. Increase in free Raptor levels does not affect mTORC1 or mTORC2 activity. mTORC1 kinase activity on 4E-BP1 substrate.

FIG. 26B. Liver Western blots of adult Ad-GFP and Ad-Raptor male mice, sacrificed after a 16 h fast followed by 4 h refeeding (n=6/group).

FIG. 26C. Protein concentration of adult Ad-GFP and Ad-Raptor male mice, sacrificed after a 16 h fast followed by 4 h refeeding (n=6/group).

FIG. 26D. Liver Western blots of adult Ad-GFP and Ad-Raptor male mice, sacrificed after a 16 h fast followed by 4 h refeeding (n=6/group).

FIG. 26E. Western blots from livers of young or adult Ad-GFP and Ad-Raptor male mice, sacrificed after a 16 h fast followed by 4 h refeeding after IP with IP with anti-mTOR antibody.

FIG. 26F. Western blots from livers of young or adult Ad-GFP and Ad-Raptor male mice, sacrificed after a 16 h fast followed by 4 h refeeding after IP without IP with anti-mTOR antibody.

FIG. 26G. Western blots from livers of young or adult Ad-GFP and Ad-Raptor male mice, sacrificed after a 16 h fast with or without 4 h refeeding.

FIG. 27A. Free Raptor reduces hepatocyte Akt activity. Western blots from liver of adult Ad-GFP or Ad-Raptor male mice, sacrificed after a 16 h fast with or without 4 h refeeding.

FIG. 27B. Free Raptor reduces hepatocyte Akt activity. Western blots from liver of adult Ad-GFP or Ad-Raptor male mice, sacrificed after a 16 h fast with or without 4 h refeeding.

FIG. 27C. Free Raptor reduces hepatocyte Akt activity. Liver mRNA expression in adult Ad-GFP and Ad-Raptor mice (n=6/group).

FIG. 27D. Free Raptor reduces hepatocyte Akt activity. Western blots from eWAT of young or adult Ad-GFP and Ad-Raptor male mice sacrificed after a 16 h fast followed by 4 h refeeding.

FIG. 27E. Free Raptor reduces hepatocyte Akt activity. Plasma insulin levels in young or adult Ad-GFP and Ad-Raptor male mice sacrificed after a 16 h fast followed by 4 h refeeding (n=6/group).

FIG. 28A. Raptor, but not mTORC1 activity, post-transcriptionally regulates PHLPP2. Western blots of PHLPP isoforms from Hepa1c1c7 cells and liver.

FIG. 28B. Raptor, but not mTORC1 activity, post-transcriptionally regulates PHLPP2. Western blots from primary hepatocytes transduced with Ad-GFP or Ad-Raptor, then treated with vehicle, Rapamycin, or Torin1.

FIG. 28C. Raptor, but not mTORC1 activity, post-transcriptionally regulates PHLPP2. Western blots from Tsc+/+ and Tsc2−/− MEFs. Blots are representative of three independent experiments.

FIG. 28D. Western blot from adult Raptor fl/fl liver transduced with AAV8-TBG-GFP or AAV8-TBG-Cre, normalized to β-actin.

FIG. 28E. Western blot from HFD-fed Raptor fl/fl liver transduced with AAV8-TBG-GFP or AAV8-TBG-Cre, normalized to β-actin.

FIG. 28F. Western blot from primary hepatocytes transduced with Ad-shControl, Ad-shRaptor, or Ad-shRictor. Blots are representative of three independent experiments.

FIG. 28G. Phlpp1 and Phlpp2 gene expression in Ad-GFP or Ad-Raptor-transduced liver.

FIG. 28H. Phlpp1 and Phlpp2 gene expression in Ad-GFP or Ad-Raptor-transduced primary hepatocytes.

FIG. 28I. Western blot of primary hepatocytes co-transduced with PHLPP1 or PHLPP2 and Ad-Raptor (or Ad-GFP control).

FIG. 28J. Western blot of Raptor (or GFP control)-transduced Hepa1c1c7 cells following immunoprecipitation with anti-PHLPP2, with or without MG-132.

FIG. 29A. PHLPP2 knockdown increases Akt S473 phosphorylation and causes hepatic steatosis. Western blot from primary hepatocytes transduced with Ad-shControl, Ad-shPhlpp1, or Ad-shPhlpp2.

FIG. 29B. PHLPP2 knockdown increases Akt S473 phosphorylation and causes hepatic steatosis. Liver weight in livers of adult Ad-shControl, Ad-shPhlpp1, or Ad-shPhlpp2 mice sacrificed after a 16 h fast followed by 4 h refeeding (n=6/group). **P<0.01 as compared to the indicated control.

FIG. 29C. PHLPP2 knockdown increases Akt S473 phosphorylation and causes hepatic steatosis. (Oil-Red-O or H&E staining in livers of adult Ad-shControl, Ad-shPhlpp1, or Ad-shPhlpp2 mice sacrificed after a 16 h fast followed by 4 h refeeding (n=6/group).

FIG. 30A. Rescue of aging/obesity-reduced PHLPP2 levels reduces hepatic steatosis. Body weight in adult, HFD-fed Ad-GFP or Ad-PHLPP2 male mice, sacrificed after a 16 h fast followed by 4 h refeeding (n=6-7/group).

FIG. 30B. Rescue of aging/obesity-reduced PHLPP2 levels reduces hepatic steatosis. Relative eWAT in adult, HFD-fed Ad-GFP or Ad-PHLPP2 male mice, sacrificed after a 16 h fast followed by 4 h refeeding (n=6-7/group).

FIG. 30C. Rescue of aging/obesity-reduced PHLPP2 levels reduces hepatic steatosis. Liver weight in adult, HFD-fed Ad-GFP or Ad-PHLPP2 male mice, sacrificed after a 16 h fast followed by 4 h refeeding (n=6-7/group). *P<0.05, **P<0.01 compared to the indicated control.

FIG. 31A. Metabolic effects of free Raptor are PHLPP2-dependent. Body weight of adult Ad-GFP and Ad-Raptor mice co-transduced with Ad-shControl, Ad-shPHLPP1 or Ad-shPHLPP2 adenoviruses, sacrificed after a 16 h fast followed by 4 h refeeding (n=6/group).

FIG. 31B. Metabolic effects of free Raptor are PHLPP2-dependent. eWAT weight of adult Ad-GFP and Ad-Raptor mice co-transduced with Ad-shControl, Ad-shPHLPP1 or Ad-shPHLPP2 adenoviruses, sacrificed after a 16 h fast followed by 4 h refeeding (n=6/group).

FIG. 31C. Metabolic effects of free Raptor are PHLPP2-dependent. Plasma β-hydroxybutyrate of adult Ad-GFP and Ad-Raptor mice co-transduced with Ad-shControl, Ad-shPHLPP1 or Ad-shPHLPP2 adenoviruses, sacrificed after a 16 h fast followed by 4 h refeeding (n=6/group).

FIG. 31D. Metabolic effects of free Raptor are PHLPP2-dependent. NEFA levels of adult Ad-GFP and Ad-Raptor mice co-transduced with Ad-shControl, Ad-shPHLPP1 or Ad-shPHLPP2 adenoviruses, sacrificed after a 16 h fast followed by 4 h refeeding (n=6/group).

FIG. 31E. Metabolic effects of free Raptor are PHLPP2-dependent. Hepatic protein concentration of adult Ad-GFP and Ad-Raptor mice co-transduced with Ad-shControl, Ad-shPHLPP1 or Ad-shPHLPP2 adenoviruses, sacrificed after a 16 h fast followed by 4 h refeeding (n=6/group).

FIG. 32A. Rescue of free Raptor or PHLPP2 does not affect glucose homeostasis. Glucose levels in young or adult Ad-GFP and Ad-Raptor male mice sacrificed after a 16 h fast with or without 4 h refeeding (n=6/group).

FIG. 32B. Rescue of free Raptor or PHLPP2 does not affect glucose homeostasis. Hepatic gluconeogenic gene expression in young or adult Ad-GFP and Ad-Raptor male mice sacrificed after a 16 h fast with or without 4 h refeeding (n=6/group).

FIG. 32C. Intraperitoneal glucose tolerance test (GTT) in young or adult Ad-GFP and Ad-Raptor mice (n=6/group).

FIG. 32D. Blood glucose in adult, HFD-fed Ad-GFP and Ad-PHLPP2 male mice, sacrificed after a 16 h fast followed by 4 h refeeding (n=6-7/group).

FIG. 32E. Plasma insulin levels in adult, HFD-fed Ad-GFP and Ad-PHLPP2 male mice, sacrificed after a 16 h fast followed by 4 h refeeding (n=6-7/group).

FIG. 33A. Hepatic PHLPP2 protein levels are declined in acute refed or aged and obese livers. Western blots from livers of fasted and re-fed mice, and normalization to β-actin

FIG. 33B. Hepatic PHLPP2 protein levels are declined in acute refed or aged and obese livers. Quantification of PHLPP1 and PHLPP2 signal.

FIG. 33C. Hepatic PHLPP2 protein levels are declined in acute refed or aged and obese livers. Western blots from livers of chow- and HFD-fed mice, and normalization to β-actin.

FIG. 33D. Hepatic PHLPP2 protein levels are declined in acute refed or aged and obese livers. Western blots from livers of lean and ob/ob mice, and normalization to β-actin.

FIG. 34. Identification of PHLPP2 protein modification. C57BL/6 mice were injected with GFP (control) or HA/Flag/PHLPP2 adenovirus and sacrificed on day 5 after overnight fast, 2 hour re-fed, or 12 hour re-fed. Immunoprecipitation (IP) with anti-Flag (harsher condition) and liquid chromatography-tandem mass spectrometry (LC/MS-MS) were performed.

FIG. 35. Phosphorylation sites identified in PHLPP2.

FIG. 36. BasePeak Chromatogram. Overlay if base peak chromatogram of trypsin digest of the bottom band (enriched in PHLPP2). To no surprise the dominant peaks are identical between the three samples.

FIG. 37. Amino acid sequence alignment of PHLPP2 among various species. Site-directed mutagenesis of Ser/Thr to Ala was performed and the phosphorylation of each mutant was tested.

FIG. 38A. Schematic of phos-tag-based mobility shift detection of phosphorylated proteins.

FIG. 38B. Phos-tag-based mobility shift detection of phosphorylated proteins in the absence of phos-tag.

FIG. 38C. Phos-tag-based mobility shift detection of phosphorylated proteins in the presence of 25 μM phos-tag.

FIG. 39. Identification of upstream kinases for PHLPP2 phosphorylation.

FIG. 40A. Identification of phosphorylation sites in PHLPP2. Each identified Ser/Thr site from LC/MS-MS-based phospho-peptide mapping results was individually mutated to Alanine, then transfected WT or Ala-mutant PHLPP2 in hepatocytes. Results in the presence of serum.

FIG. 40B. Identification of phosphorylation sites in PHLPP2. Each identified Ser/Thr site from LC/MS-MS-based phospho-peptide mapping results was individually mutated to Alanine, then transfected WT or Ala-mutant PHLPP2 in hepatocytes. Results in the absence of serum.

FIG. 41A. Forskolin induced phosphorylation overexpressed PHLPP2 in primary hepatocytes.

FIG. 41B. cAMP activator induces PHLPP2 phosphorylation on two Serine residues. WT, S1119A, and S1210A mutant of PHLPP2 were all phosphorylated with forskolin treatment.

FIG. 41C. cAMP activator induces PHLPP2 phosphorylation on two Serine residues. Ablation of both sites rendered the S1119A/S1210A mutant insensitive to forskolin-induced phosphorylation.

FIG. 42A. Identification of phosphorylation sites in PHLPP2 by Glucagon/PKA signaling. Forskolin-mediated PHLPP2 phosphorylation.

FIG. 42B. Identification of phosphorylation sites in PHLPP2 by Glucagon/PKA signaling. Forskolin-mediated PHLPP2 phosphorylation was reduced by pretreatment of PKA inhibitor, H89.

FIG. 43A. Inhibition of Glucagon signaling increases PHLPP2 protein levels in db/db mice. Glucagon receptor (Gcgr) expression from livers of db/db mice transduced with AAV8-shControl or AAV8-shGcgr.

FIG. 43B. Inhibition of Glucagon signaling increases PHLPP2 protein levels in db/db mice. Western blots from livers of db/db mice transduced with AAV8-shControl or AAV8-shGcgr.

FIG. 43C. Inhibition of Glucagon signaling increases PHLPP2 protein levels in db/db mice. Quantification of Western blots in FIG. 43B, normalized to either total PHLPP2 or β-actin.

FIG. 44A. Inhibition of Glucagon signaling increases PHLPP2 protein levels in DIO mice. Western blots from livers of diet-induced obese (DIO) C57BL6 mice transduced with either AAV8-shControl or AAVB-shGcgr.

FIG. 44B. Inhibition of Glucagon signaling increases PHLPP2 protein levels in DIO mice. Quantification of PHLPP2 levels, normalized to β-actin.

FIG. 45A. Generation of PHLPP2 KO cells using Genome-scale CRISPR Knock-Out (GeCKO) system. PHLPP2 KO HepG2 hepatoma cells were generated using the CRISPR (Clustered Regularly Interspaced Short Palindromic Repeats)-Cas9 system. Using three different single guide RNA (sgRNA) for PHLPP2, significantly reduced PHLPP2 protein and mRNA levels

FIG. 45B. Generation of PHLPP2 KO cells using Genome-scale CRISPR Knock-Out (GeCKO) system. Quantification of PHLPP2 levels in FIG. 45A.

FIG. 45C. Generation of PHLPP2 KO cells using Genome-scale CRISPR Knock-Out (GeCKO) system. Quantification of off-target gene expression.

FIG. 46A. Generation of PHLPP2 KO cells using Genome-scale CRISPR Knock-Out (GeCKO) system. HepG2 stable cell lines were generated expressing the most efficient PHLPP2 sgRNA (#3) sgRNA3.

FIG. 46B. Generation of PHLPP2 KO cells using Genome-scale CRISPR Knock-Out (GeCKO) system. Mouse hepatoma cell lines, Hepa1c1c7 cells, were generated.

FIG. 47A. Characterization of PHLPP2 KO cells. PHLPP2-deficient cells showed sustained insulin signaling.

FIG. 47B. Characterization of PHLPP2 KO cells. PHLPP2 levels “rescued” in CRISPR-induced knockout cell lines with either WT or S1119/1210A PHLPP2.

FIG. 48. Identification of novel interaction partners with PHLPP2.

FIG. 49A. Identification of novel interaction partners with PHLPP2-KCTD17 interaction with PHLPP2. PHLPP2 associated with KCTD17 in a proteasome-dependent manner.

FIG. 49B. Identification of novel interaction partners with PHLPP2-KCTD17 interaction with PHLPP2. Association of endogenous PHLPP2 with KCTD17 was significantly enhanced by treatment of forskolin and simultaneous treatment with forskolin and the proteasome inhibitor, MG132.

FIG. 50. Identification of novel interaction partners with PHLPP2-KCTD17 interaction with PHLPP2, phosphorylation-dependent manner.

FIG. 51 Notch loss-of-function—genetic approaches.

FIG. 52A. Notch inhibitors reduce obesity-induced fatty liver.

FIG. 52B. Notch inhibitors reduce obesity-induced glucose intolerance.

FIG. 53A. Activation of hepatocyte Notch causes glucose intolerance.

FIG. 53B. Activation of hepatocyte Notch causes fatty liver.

FIG. 54A. Notch-induced fatty liver can be prevented with the mTORC1 inhibitor rapamycin.

FIG. 54B. Notch-induced glucose intolerance cannot be prevented with the mTORC1 inhibitor rapamycin.

FIG. 55. Working model: Notch increases hepatic glucose and lipid production.

FIG. 56. Hepatic Notch activity correlated with hepatic Raptor levels without change in mRNA levels.

FIG. 57. Hypothesis: Raptor overexpression will “rescue” decreased liver TG in L-Rbpj mice.

FIG. 58. Raptor lowers hepatic TG in control mice.

FIG. 59. Experiment repeated using wild-type mice.

FIG. 60. No change in mTORC1 target phosphorylation, kinase activity, or hepatic protein content.

FIG. 61. Theft of common components of mTORC1 and mTORC2 complexes was hypothesized, however no change in mTORC2 activity was determined.

FIG. 62. Raptor overexpression terminates insulin action by reducing Akt activity.

FIG. 63A. Free Raptor reduces Akt activity. No change in insulin, hepatic pIR/p-IRS.

FIG. 63B. Free Raptor reduces Akt activity to inhibit lipogenesis, and fatty liver. Aging was associated with a trend towards higher expression of key lipogenic genes, Srebp1c, Fasn (fatty acid synthase), Acc1 (acetyl-CoA carboxylase 1), and Scd1 (steroyl-CoA desaturase 1), which was reversed by increased free Raptor.

FIG. 63C. Free Raptor reduces Akt activity to inhibit lipogenesis, and fatty liver. Aging was associated with increased DNL, which was reversed by increased free Raptor.

FIG. 63D. Free Raptor reduces Akt activity to inhibit lipogenesis, and fatty liver. Ad-Raptor reduced TG content.

FIG. 64A. Aging/obesity leads to decreased hepatic PHLPP2. Western blots from livers of young and adult mice, and normalization to β-actin.

FIG. 64B. Aging/obesity leads to decreased hepatic PHLPP2. Western blots from livers of chow- and HFD-fed mice, and normalization to β-actin.

FIG. 64C. Aging/obesity leads to decreased hepatic PHLPP2. Western blots from livers of lean and ob/ob mice, and normalization to β-actin.

FIG. 64D. Aging/obesity leads to decreased hepatic PHLPP2. PHLPP1 and PHLPP2 protein levels from control, aged, HFD-fed, and ob/ob mice.

FIG. 64E. Aging/obesity leads to decreased hepatic PHLPP2. Western blot from liver of Ad-GFP and Ad-Raptor mice.

FIG. 65. Model—Insulin resistance.

### DETAILED DESCRIPTION OF THE INVENTION

Embodiments of the Invention

The present invention provides a method of reducing a subject's hepatic and plasma triglyceride levels comprising administering to a subject in need thereof a pharmaceutical composition comprising a pharmaceutical carrier and a compound that increases PHLPP2 in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that increases free Raptor in liver cells in an amount effective to reduce the subject's hepatic and plasma triglyceride levels.

In some embodiments, the pharmaceutical composition increases Raptor expression, thereby increasing free Raptor in the liver cells.

In some embodiments, the pharmaceutical composition inhibits interaction of Raptor and mTORC1, thereby increasing free Raptor in the liver cells.

In some embodiments, the compound reduces the expression of at least one lipogenic gene.

In some embodiments, the at least one lipogenic gene is Srebp1c, Fasn, Acc1, or Scd1.

In some embodiments, the subject is afflicted with a metabolic disease.

In some embodiments, the pharmaceutical composition comprises a polynucleotide.

In some embodiments, the pharmaceutical composition is targeted to the liver of the subject.

In some embodiments, the metabolic disease is obesity.

In some embodiments, the metabolic disease is hypertriglyceridemia.

In some embodiments, the metabolic disease is hyperinsulinemia.

In some embodiments, the metabolic disease is Type 2 Diabetes.

In some embodiments, the metabolic disease is fatty liver disease.

In some embodiments, the fatty liver disease is nonalcoholic fatty liver disease or nonalcoholic steatohepatitis.

In some embodiments, the subject is afflicted with cirrhosis or hepatocellular carcinoma.

In some embodiments, the subject is a human.

In some embodiments, the subject's hepatic or plasma triglyceride levels are >150 mg/dL.

In some embodiments, the subject's hepatic or plasma triglyceride levels are >500 mg/dL, about 200 to 499 mg/dL, or about 150 to 199 mg/dL.

In some embodiments, the subject's hepatic or plasma triglyceride levels are reduced by at least 20%, at least 25%, at least 30%, at least 35%, at least 40%, at least 45%, at least 50%, at least 55%, at least 60%, at least 65%, at least 70%, or at least 75%, relative to the level prior to the administration.

The present invention also provides a process for determining the amount of free Raptor in a subject's liver comprising:
d) obtaining a biological sample comprising liver cells of the subject;
e) separating free Raptor and mTORC1-associated Raptor in the sample; and
f) determining the amount of free Raptor in the sample.

The present invention also provides a process for diagnosing whether a subject is afflicted with decreased free Raptor comprising:
a) determining the amount of free Raptor in the subject according to the process of the claimed invention;
b) determining the amount of free Raptor in a reference subject according to the process of the claimed invention; and
d) diagnosing the subject to be afflicted with decreased free Raptor if the amount of free Raptor in step (a) is substantially decreased compared to the amount of free Raptor in step (b).

The present invention also provides a method of treating a subject diagnosed to be afflicted with decreased free Raptor according to the process of the claimed invention comprising reducing the subject's hepatic and plasma triglyceride levels according to the method of the claimed invention.

In some embodiments, the pharmaceutical composition reduces β-TrCP-mediated degradation of PHLPP2, thereby increasing free Raptor in the liver cells.

In some embodiments, the pharmaceutical composition comprises a Notch antagonist.

In some embodiments, the Notch antagonist comprises a γ-secretase inhibitor, anti-DLL4 mAB, or a DLK peptide.

In some embodiments, the pharmaceutical composition decreases PHLPP2 phosphorylation at Serine 1119 or Serine 1210 residues.

In some embodiments, the pharmaceutical composition inhibits interaction of PHLPP2 and KCTD17.

In some embodiments, the pharmaceutical composition decreases Akt signaling.

In some embodiments, the pharmaceutical composition decreases Akt phosphorylation at Serine 473 residue.

In some embodiments, the pharmaceutical composition prevents PHLPP2 degradation by inhibiting Glucagon signaling.

In some embodiments, the pharmaceutical composition reduces PHLPP2 degradation, thereby increasing free Raptor in the liver cells.

In some embodiments, the pharmaceutical composition prevents PHLPP2 degradation, thereby increasing free Raptor in the liver cells.

Methods of Inhibiting Glucagon Signaling

As used herein, “inhibiting glucagon signaling” includes reducing glucagon receptor expression, reducing glucagon receptor activation, blocking glucagon receptor activation, reducing endogenous glucagon action, or blocking glucagon action.

As used herein, “prevents PHLPP2 degradation” includes inhibiting PHLPP2 phosphorylation, thereby increasing PHLPP2 levels compared to controls.

As used herein, “reduces PHLPP2 degradation” includes inhibiting PHLPP2 phosphorylation, thereby increasing PHLPP2 levels compared to controls.

In some embodiments, the pharmaceutical composition inhibits Glucagon signaling, thereby reducing PHLPP2 degradation.

In some embodiments, the pharmaceutical composition comprises a Glucagon receptor (Gcgr) antagonizing antibody.

In some embodiments, the pharmaceutical composition comprises a Glucagon receptor (Gcgr) antagonist.

In some embodiments, the Glucagon receptor antagonist reduces PHLPP2 degradation.

In some embodiments, the Glucagon receptor antagonist is des-His¹-[Glu⁹]-Glucagon (1-29) amide, or L-168,049.

In some embodiments, the Glucagon receptor antagonist is 4-[3-(5-Bromo-2-propoxyphenyl)-5-(4-chlorophenyl)-1H-pyrrol-2-yl]pyridine.

In some embodiments, glucagon is suppressed by somatostatin infusion.

The present invention also provides a method of reducing a subject's hepatic and plasma triglyceride levels comprising administering to a subject in need thereof a pharmaceutical composition comprising a pharmaceutical carrier and a compound that prevents PHLPP2 degradation in liver cells in an amount effective to reduce the subject's hepatic and plasma triglyceride levels.

The present invention also provides a method of reducing a subject's hepatic and plasma triglyceride levels comprising administering to a subject in need thereof a pharmaceutical composition comprising a pharmaceutical carrier and a compound that inhibits Glucagon signaling in liver cells in an amount effective to reduce the subject's hepatic and plasma triglyceride levels.

In some embodiments, the pharmaceutical composition prevents PHLPP2 degradation, thereby increasing free Raptor in the liver cells.

In some embodiments, each compound administered to the subject is, independently, an organic compound having a molecular weight less than 1000 Daltons, a DNA aptamer, an RNA aptamer, a polypeptide, an antibody, an oligonucleotide, an interfering RNA (RNAi) molecule, a ribozyme, or a small molecule inhibitor.

In some embodiments, a compound that is capable of inhibiting glucagon signaling is administered to the subject.

In some embodiments, the compound which is capable of inhibiting glucagon signaling is an organic compound having a molecular weight less than 1000 Daltons.

In some embodiments, the oligonucleotide is an antisense oligonucleotide, an RNA-interference inducing compound, or a ribozyme.

In some embodiments, the oligonucleotide is targeted to hepatocytes.

In some embodiments, the oligonucleotide comprises 1, 2, 3, 4, or 5 or more stretches of nucleotides in a sequence that is complementary to glucagon-encoding mRNA or glucogon receptor-encoding mRNA, wherein each stretch of complementary continguous nucleotides is at least at least 4, 5, 6, 7, 8, 9, 10, 11, 12, 13, 14, 15, 16, 17, 18, 19, 20, 21, 22, 23, 24, 25 or more nucleotides in length.

In some embodiments, the oligonucleotide is modified to increase its stability in vivo.

Aspects of the present invention relate to the use of compounds effective to increase free RAPTOR can be used treat metabolic diseases such as hypertriglyceridemia, hyperinsulinemia, Type 2 Diabetes, or fatty liver disease.

Aspects of the present invention relate to the use of compounds effective to increase PHLPP2 can be used treat metabolic diseases such as hypertriglyceridemia, hyperinsulinemia, Type 2 Diabetes, or fatty liver disease.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Methods

Experimental Animals

Male wild-type C57/BL/6 mice, fed on standard chow or HFD (Harlan Laboratories TD.06414), were purchased from Jackson Labs. We injected AAV8-TBG-GFP or AAV8-TBG-Cre (Penn Vector Core) into male Raptorfl/fl (Jackson Laboratory; stock number 013188) mice, and characterized the mice 2 weeks after adeno-associated virus (AAV) injection. Number of animals used in experiments was chosen to ensure adequate power to detect experimental difference with alpha set to 0.05. All animal experiments were conducted in accordance with guidelines of the Columbia University Institutional Animal Care and Use Committee.

Metabolic Analyses

Blood glucose was measured using a glucose meter (OneTouch), and plasma insulin by mouse insulin ELISA kit (Mercodia). Glucose tolerance tests were performed by intraperitoneal injection of 2 g per kg body weight glucose after a 16 h fast. Hepatic lipids were extracted (Folch et al., 1957), and plasma and hepatic triglyceride, Cholesterol E and NEFA were measured using a colorimetric assay from Thermo or Wako Chemicals, according to the manufacturer's protocol. We measured de novo lipogenesis as previously described (Zhang et al., 2006).

Adenovirus Studies

The GFP, Raptor and PHLPP1/2 adenoviruses have been described (Miyamoto et al., 2010; Pajvani et al., 2013). The HA-Flag-PHLPP2 adenovirus was constructed by inserting a Flag sequence into HA-PHLPP2 vector (Addgene #22403, courtesy of Alexandra Newton) and adenoviruses encoding HA-Flag-PHLPP2 generated by Welgen, Inc. (Worcester, Mass.). Mouse shPhlpp1 or 2 target sequences were selected among three candidate sequences, respectively and adenoviruses encoding shPhlpp1 or 2 generated by Welgen, Inc. For in vivo studies, we injected 5×108 or 2.5×108 purified viral particles per g body weight; we performed metabolic analysis on days 3-5 and euthanized the mice at day 8 or 10 after injection. Infection with adenovirus in primary hepatocytes or Hepa1c1c7 cells was performed at 2.5, 5, or 10 multiplicity of infection (MOI).

Primary Hepatocyte Cultures

We isolated and cultured primary mouse hepatocytes as previously described (Kim et al., 2012). For inhibitor experiments, we treated hepatocytes with 20 nM rapamycin (Cell Signaling), 250 nM Torin1, or vehicle for either 1 h or 24 h.

Western Blotting and Immunoprecipitation

Tissues and cells were lysed in 0.3% CHAPS lysis buffer (Kim et al., 2002) unless otherwise stated, and whole cell lysates obtained by centrifugation. Immunoblots were conducted on samples randomly chosen within each experimental cohort with antibodies against Raptor, Akt, p-Akt (S473), p-Akt (T308), p-Akt1 (S473), p-Akt2 (S474), p-Akt (T450), p-Akt substrate, PKCα, p-GSK3β (S9), GSK3β, mTOR, Rictor, GβL, p-S6 (S240/244), S6, β-TrCP and β-actin from Cell Signaling; mTOR and α-tubulin from SantaCruz Biotechnology Inc.; PHLPP1 and PHLPP2 from Bethyl Laboratories Inc.; as well as a polyclonal antibody against Raptor from Invitrogen. For immunoprecipitation (IP) experiments, liver or cellular lysate was incubated with anti-mTOR or PHLPP2 antibody immobilized on Protein A or G-Sepharose (Invitrogen).

Gel Filtration Chromatography

Livers from three mice per group were pooled and lysed in hypotonic buffer (40 mM Tris-HCl, pH 7.5) with protease inhibitor (Pierce) by repeated passage in a 27-gauge needle. Resultant lysates were repeatedly centrifuged at 14,000 rpm for 15 min, and the supernatant fraction filtered through a PVDF membrane prior to application to a Superose 6 column (Amersham Biosciences) calibrated with the Gel Filtration Marker Kit (Sigma), and eluted with 40 mM Tris-HCl (pH 7.5) and 150 mM NaCl.

Crosslinking Assays

Liver was lysed in 1% Triton X-100 lysis buffer (Kim et al., 2002) with or without 2.5 mg/ml DSP or DSS, and incubated for 2 h on ice. Crosslinking was quenched with 1 M Tris-HCl (pH 7.5) for 30 min on ice. For in vitro crosslinking assays, DSP or DSS were added to a final concentration of 1 mg/ml culture medium, and quenched by Tris-HCl as above. Equal protein (BCA assay, Thermo) was then subjected to Western blot, with or without IP.

Quantitative RT-PCR

We isolated RNA with TRIzol (Invitrogen) or an RNeasy Mini kit (Qiagen), synthesized cDNA with High-Capacity cDNA Reverse Transcription kit (Applied Biosystems) and performed quantitative RT-PCR with a GoTaq SYBR Green qPCR kit (Promega) in a CFX96 Real-Time PCR detection system (Bio-Rad).

In Vitro Akt Kinase Assay

Liver lysates were immunoprecipitated with anti-Akt antibody (sepharose bead conjugated); bound protein was incubated with a recombinant GSK-3 fusion protein substrate in kinase buffer for 30 min at 30° C., prior to termination with SDS loading buffer and SDS-PAGE, with p-GSK3β (S9) detected by immunoblot.

Statistical Analysis

We performed comparisons using two-way ANOVA. All data are shown as the means±s.e.m.

Terms

As used herein, “about” in the context of a numerical value or range means ±10% of the numerical value or range recited or claimed, unless the context requires a more limited range.

As used herein, “a subject afflicted with” a disease, e.g. nonalcoholic fatty liver disease, means a human patient who was been affirmatively diagnosed to have the disease.

As used herein, “effective” when referring to an amount of a compound or compounds refers to the quantity of the compound or compounds that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the physical condition of the patient, the type of subject being treated, the duration of the treatment, the nature of concurrent therapy (if any), and the specific formulations employed and the structure of the compounds or its derivatives.

In some embodiments, “a subject in need” includes a subject with elevated triglyceride levels, e.g., a subject with hepatic or plasma or serum triglyceride levels greater than 150 mg/dL, greater than 200 mg/dL, or to greater than 500 mg/dL.

In some embodiments, “a subject in need” includes a subject with decreased free Raptor, e.g., a subject afflicted with obesity.

In some embodiments, “a subject in need” encompasses, e.g., a subject with plasma or serum triglyceride level greater than 150 mg/dL, greater than 200 mg/dL, or to greater than 500 mg/dL.

In some embodiments, a subject afflicted with a metabolic disease, such as obesity, tends to have elevated triglyceride levels.

In some embodiments, a pharmaceutical composition comprises a pharmaceutical carrier and a compound.

In some embodiments, the pharmaceutical composition is manufactured, wherein the pharmaceutical composition comprises an isolated or purified naturally occurring compound.

In some embodiments, the pharmaceutical composition is manufactured, wherein the pharmaceutical composition comprises a compound which was not produced by a process in nature.

In some embodiments, a reference subject includes a subject with hepatic or plasma or serum triglyceride levels less than 150 mg/dL.

As described herein, young mice (9-week old, equivalent to an immediately post-pubertal human) tend to have “normal” Raptor levels, which decline by 24-weeks (equivalent to adulthood).

In some embodiments, a reference subject includes a subject that has not reached adulthood.

In some embodiments, a reference subject includes a subject younger than 18 years old.

In some embodiments, a reference subject is not afflicted with a metabolic disease and is younger than 18 years old.

Methods of Increasing Raptor or PHLPP2

In some embodiments, each compound administered to the subject is, independently, an organic compound having a molecular weight less than 1000 Daltons, a polypeptide, an oligonucleotide, or a small molecule.

In some embodiments, the pharmaceutical composition which is capable of increasing PHLPP2 or free Raptor enhances expression of a gene or enhances transcription.

In some embodiments, the pharmaceutical composition which is capable of increasing free Raptor inhibits interaction of Raptor and mTORC1.

In some embodiments, a pharmaceutical composition that is capable of increasing free Raptor is administered to the subject.

In some embodiments, a pharmaceutical composition that is capable of increasing PHLPP2 is administered to the subject.

In some embodiments, a pharmaceutical composition that is capable of increasing Raptor expression is administered to the subject.

In some embodiments, a pharmaceutical composition that is capable of increasing PHLPP2 expression is administered to the subject.

Small Molecule

A small molecule may be administered herein to increase free Raptor or increase activity of Raptor.

A small molecule may be administered herein to increase PHLPP2 or increase activity of PHLPP2.

Non-limiting examples of PHLPP2 activators such as anti-miRNA oligonucleotides to miR-190, or small-molecular or other inhibitors of the B-TrCP degradative pathway are described, for example, in the following publications: Beezhold, et al, Toxicological Sciences, 2011, and Li, Liu and Gao, Mol Cell Biology, 2009, which are hereby incorporated by reference in their entireties.

Oligonucleotide

Non-limiting examples of oligonucleotides capable of increasing Raptor or PHLPP2 expression include antisense oligonucleotides, polynucleotides, and adenoviral vectors.

The amino acid sequence of Raptor, or KIAA1303, is accessible in public databases by the GenBank accession number Q8N122.1, and is set forth herein as SEQ ID NO: 1.

The amino acid sequence of, PH domain leucine-rich repear-containing protein phosphatase 2 (PHLPP2), is accessible in public databases by the GenBank accession number Q6ZVD8.3, and is set forth herein as SEQ ID NO: 2.

In some embodiments, the pharmaceutical composition which is capable of increasing free Raptor or PHLPP2 comprises a polynucleotide or an adenovirus.

In some embodiments, the pharmaceutical composition which is capable of increasing Raptor or PHLPP2 expression comprises a polynucleotide or an adenovirus.

Non-limiting examples of oligonucleotides capable of decreasing Glucagon signaling include antisense oligonucleotides, polynucleotides, and adenoviral vectors.

The sequence of, Glucagon (GCG), is accessible in public databases by the GenBank accession number NM_002054.4, and is set forth herein as SEQ ID NO: 3.

The amino acid sequence of Glucagon Receptor (gcgr) is accessible in public databases by the GenBank accession number L20316.1, and is set forth herein as SEQ ID NO: 4.

As used herein, “a subject afflicted with” a disease, e.g. nonalcoholic fatty liver disease, means a human patient who was been affirmatively diagnosed to have the disease.

As used herein, “a subject afflicted with” a disease, e.g. nonalcoholic fatty liver disease, means a human patient who was been affirmatively diagnosed to have the disease.

Antisense Oligonucleotide

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of target gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). An antisense oligonucleotide also can be designed to block translation of mRNA by preventing the transcript from binding to ribosomes.

Precise complementarity is not required for successful complex formation between an antisense oligonucleotide and the complementary sequence of a target polynucleotide. Antisense oligonucleotides which comprise, for example, 1, 2, 3, 4, or 5 or more stretches of contiguous nucleotides which are precisely complementary to a target polynucleotide, each separated by a stretch of contiguous nucleotides which are not complementary to adjacent nucleotides, can provide sufficient targeting specificity for a target mRNA. Preferably, each stretch of complementary contiguous nucleotides is at least 4, 5, 6, 7, 8, 9, 10, 11, 12, 13, 14, 15, 16, 17, 18, 19, 20, 21, 22, 23, 24, 25, or more nucleotides in length. Noncomplementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular target polynucleotide sequence. Antisense oligonucleotides can be modified without affecting their ability to hybridize to a target polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art.

Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to mRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease target gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional teminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

RNA Interference

An interfering RNA (RNAi) molecule involves mRNA degradation. The use of RNAi has been described in Fire et al., 1998, Carthew et al., 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to a siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

RNAi is a powerful tool for in vitro and in vivo studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Multiple mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

Another mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

Another mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects.

The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

Isolated RNA molecules can mediate RNAi. That is, the isolated RNA molecules of the present invention mediate degradation or block expression of mRNA that is the transcriptional product of the gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

In some embodiments, an RNAi molecule of the invention is introduced into a mammalian cell in an amount sufficient to attenuate target gene expression in a sequence specific manner. The RNAi molecules of the invention can be introduced into the cell directly, or can be complexed with cationic lipids, packaged within liposomes, or otherwise delivered to the cell. In certain embodiments the RNAi molecule can be a synthetic RNAi molecule, including RNAi molecules incorporating modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications. In some embodiments, an RNAi molecule of the invention contains modified nucleotides that increase the stability or half-life of the RNAi molecule in vivo and/or in vitro. Alternatively, the RNAi molecule can comprise one or more aptamers, which interact(s) with a target of interest to form an aptamer:target complex. The aptamer can be at the 5′ or the 3′ end of the RNAi molecule. Aptamers can be developed through the SELEX screening process and chemically synthesized. An aptamer is generally chosen to preferentially bind to a target. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23.

Adenoviral Vector

An adenoviral vecor encodes an oligonucleotide. The use of adenoviral vectors in gene therapy and tissue-specific targeting has been described in Beatty and Curiel, 2012, Barnett et al., 2002, and Rots et al., 2003, the contents of which are incorporated herein by reference.

Methods of Administration

“Administering” compounds in embodiments of the invention can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject.

Injectable Drug Delivery

Injectable drug delivery systems may be employed in the methods described herein include solutions, suspensions, gels.

Oral Drug Delivery

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

For oral administration in liquid dosage form, a PHLPP2 or Raptor activator may be combined with any oral, non-toxic, pharmaceutically acceptable inert carrier such as ethanol, glycerol, water, and the like.

Pharmaceutically Acceptable Carrier

The compounds used in embodiments of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes such as small unilamellar vesicles, large unilamallar vesicles, and multilamellar vesicles are also a pharmaceutically acceptable carrier. Liposomes can be formed from a variety of phospholipids, such as cholesterol, stearylamine, or phosphatidylcholines. The compounds may be administered as components of tissue-targeted emulsions. Examples of lipid carriers for antisense delivery are disclosed in U.S. Pat. Nos. 5,855,911 and 5,417,978, which are incorporated herein by reference. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

A compound of the invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297, issued Sept. 2, 1975.

Tablets

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Specific Administration to Liver

Embodiments of the invention relate to specific administration to the liver or hepatocytes.

In some embodiments, a compound may specifically target the liver.

In some embodiments, a compound may specifically target hepatocytes.

In some embodiments, a compound may be specifically targeted to the liver by coupling the compound to ligand molecules, targeting the compound to a receptor on a hepatic cell, or administering the compound by a bio-nanocapsule.

A compound of the invention can also be administered by coupling of ligand molecules, such as coupling or targeting moieties on preformed nanocarriers, such as (PGA-PLA nanoparticles, PLGA nanoparticles, cyclic RGD-doxorubicin-nanoparticles, and poly(ethylene glycol)-coated biodegradable nanoparticles), by the post-insertion method, by the Avidin-Biotin complex, or before nanocarriers formulation, or by targeting receptors present on various hepatic cell, such as Asialoglycoproein receptor (ASGP-R), HDL-R, LDL-R, IgA-R, Scavenger R, Transferrin R, and Insulin R, as described in: Mishra et al., (2013) Efficient Hepatic Delivery of Drugs: Novel Strategies and Their Significance, BioMed Research International 2013: 382184, dx.doi.org/10.1155/2013/382184, the entire contents of which are incorporated herein by reference.

A compound of the invention can also be administered by bio-nanocapsule, as described in: Yu et al., (2005) The Specific delivery of proteins to human liver cells by engineered bio-nanocapsules, FEBS Journal 272: 3651-3660, dx.doi.org/10.1111/j.1742-4658.2005.04790.x, the entire contents of which are incorporated herein by reference.

In some embodiments, an oligonucleotide specifically targets the liver.

In some embodiments, an oligonucleotide specifically targets hepatocytes.

Antisense oligonucleotides of the invention can also be targeted to hepatocytes, as described in: Prakash et al., (2014) Targeted delivery of antisense oligonucleotides to hepatocytes using triantennary N-acetyl galactosamine improves potency 10-fold in mice, Nucleic Acids Research 42(13): 8796-8807, dx.doi.org/10.1093/nar/gku531, the entire contents of which are incorporated herein by reference.

As used herein, the term “effective amount” refers to the quantity of a component that is sufficient to treat a subject without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of subject being treated, the duration of the treatment, the nature of concurrent therapy (if any), and the specific formulations employed and the structure of the compounds or its derivatives.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The dosage of a compound of the invention administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the invention may comprise a compound alone, or mixtures of a compound with additional compounds used to treat cancer. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

In an embodiment, the pharmaceutical composition may be administered once a day, twice a day, every other day, once weekly, or twice weekly.

A subject's triglyceride level may be expressed herein as hepatic triglyceride or plasma triglyceride or serum triglyceride.

Where a range is given in the specification it is understood that the range includes all integers and 0.1 units within that range, and any sub-range thereof. For example, a range of 1 to 5 is a disclosure of 1.0, 1.1, 1.2, etc.

This invention will be better understood by reference to the Examples which follow, but those skilled in the art will readily appreciate that the specific experiments detailed are only illustrative of the invention as described more fully in the claims which follow thereafter.

### Example 1

“Free” Raptor Levels Decline in Aged and Obese Liver

Although mTORC1 activity is canonically and obligatorily activated by nutrient availability (Kim et al., 2002), another layer of regulation has recently been demonstrated at the level of mTORC1 complex stability (Pajvani et al., 2013). To clarify the mechanism underlying mTOR component association in liver, size-exclusion chromatography was used to fractionate detergent-free liver lysate from young, chow-fed mice. Interestingly, while the vast majority of mTOR and GβL protein was found in high-molecular weight (^{˜}800 kDa) fractions, consistent with mTORC1 complex dimers (Jain et al., 2014; Menon et al., 2014; Yip et al., 2010), Raptor is distributed evenly in mTORC1-associated and -free Raptor (^{˜}150 kDa) fractions (FIGS. 1A and 1B). As mTORC1 function in liver is known to be modulated by aging and diet—L-Raptor mice show aging-dependent defects in ketogenesis (Sengupta et al., 2010), as well as reduced hepatic triglyceride (TG) in response to Western-type diet feeding (Peterson et al., 2011)—it was hypothesized that mTORC1 complex assembly may differentially be affected in these pathophysiologic processes. Indeed, livers from older or leptin-deficient (ob/ob) obese mice showed unchanged size distribution of mTOR and GβL, but a marked reduction of free Raptor (FIGS. 1A and 1B).

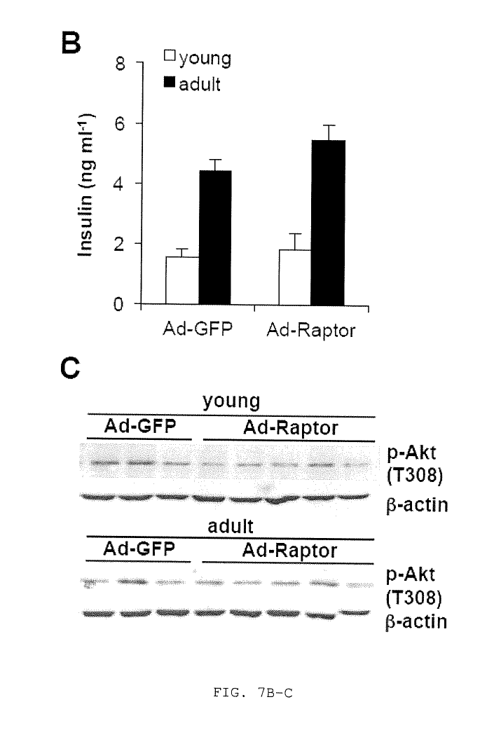

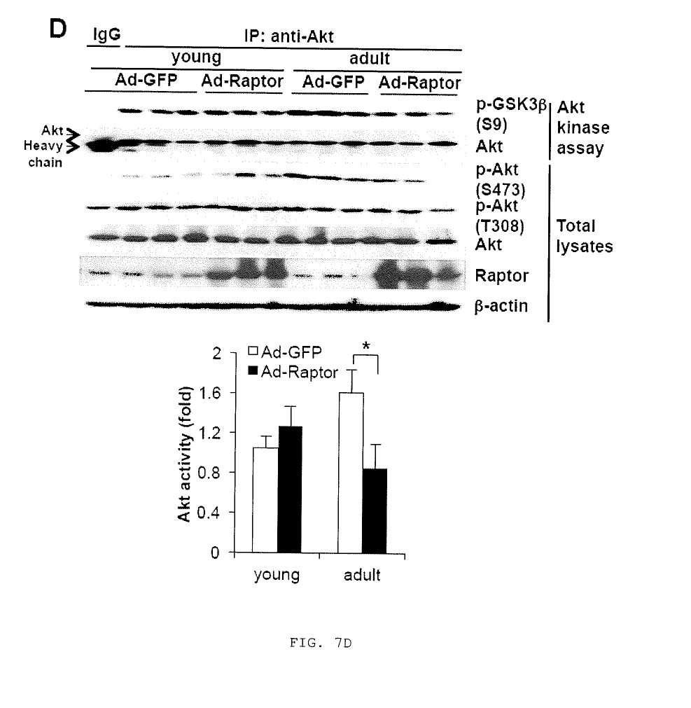

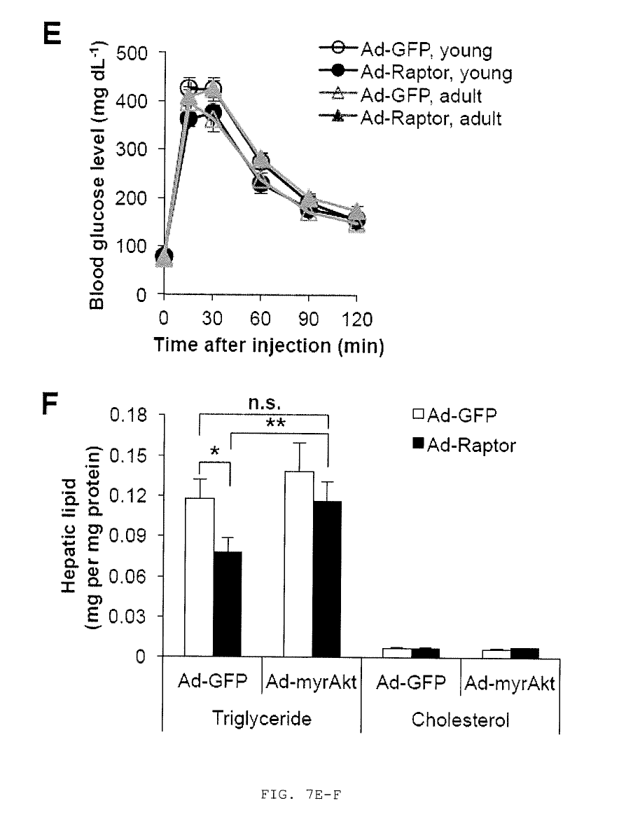

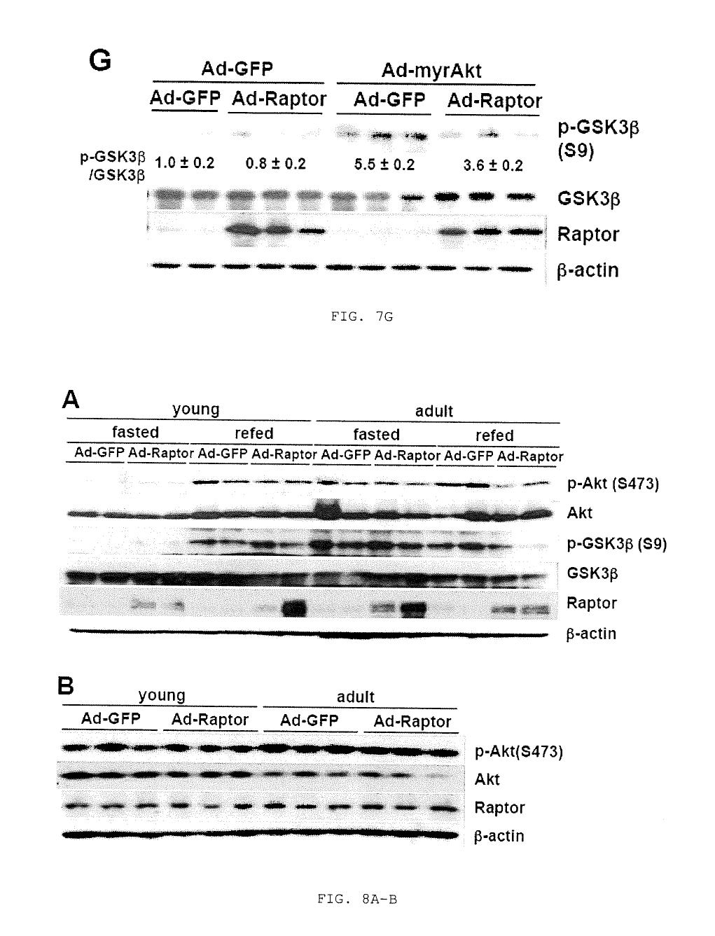

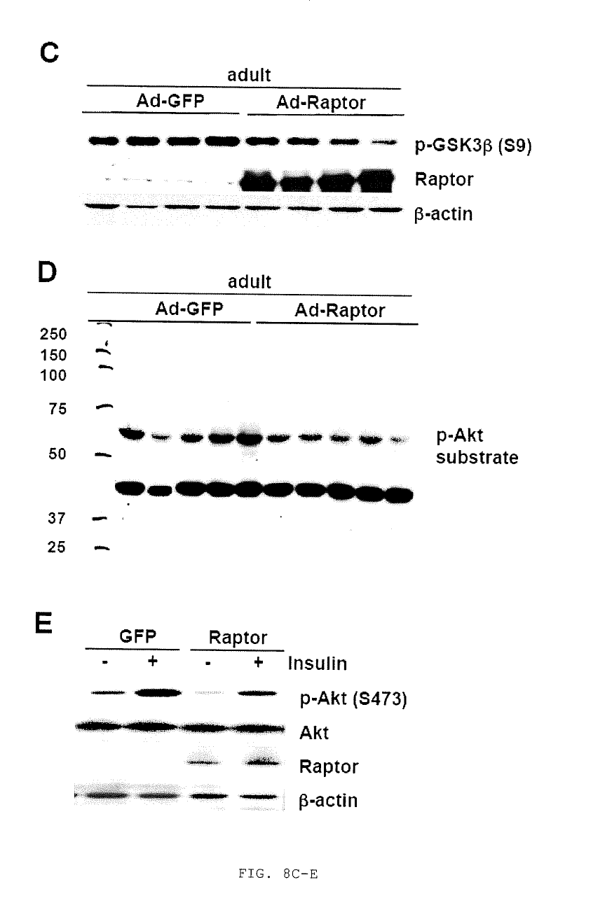

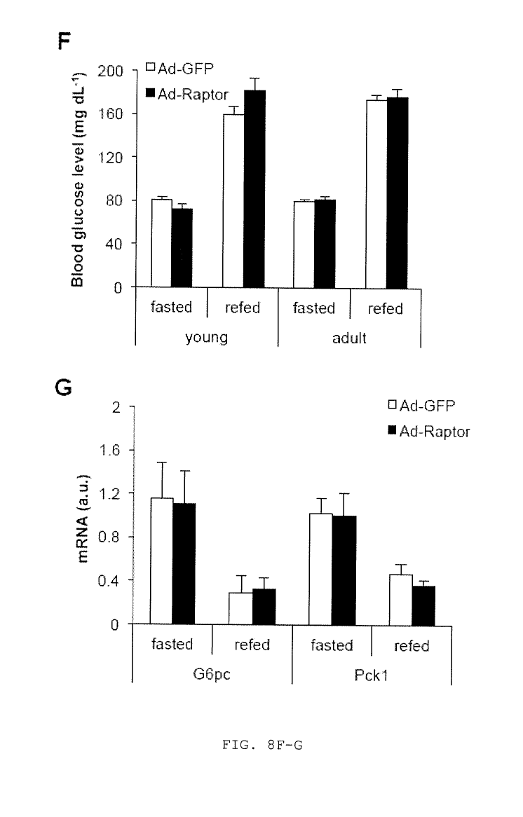

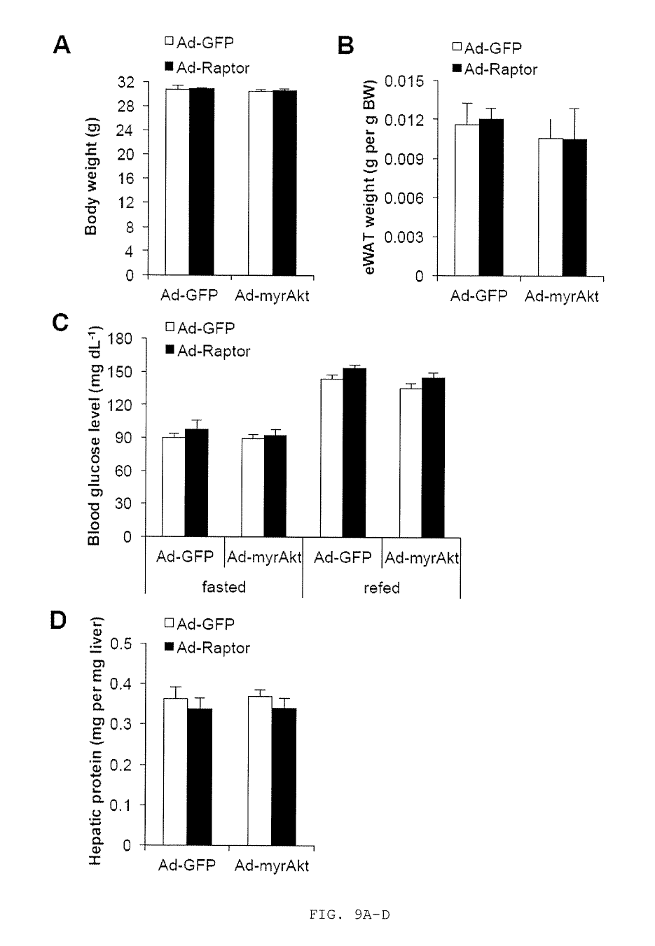

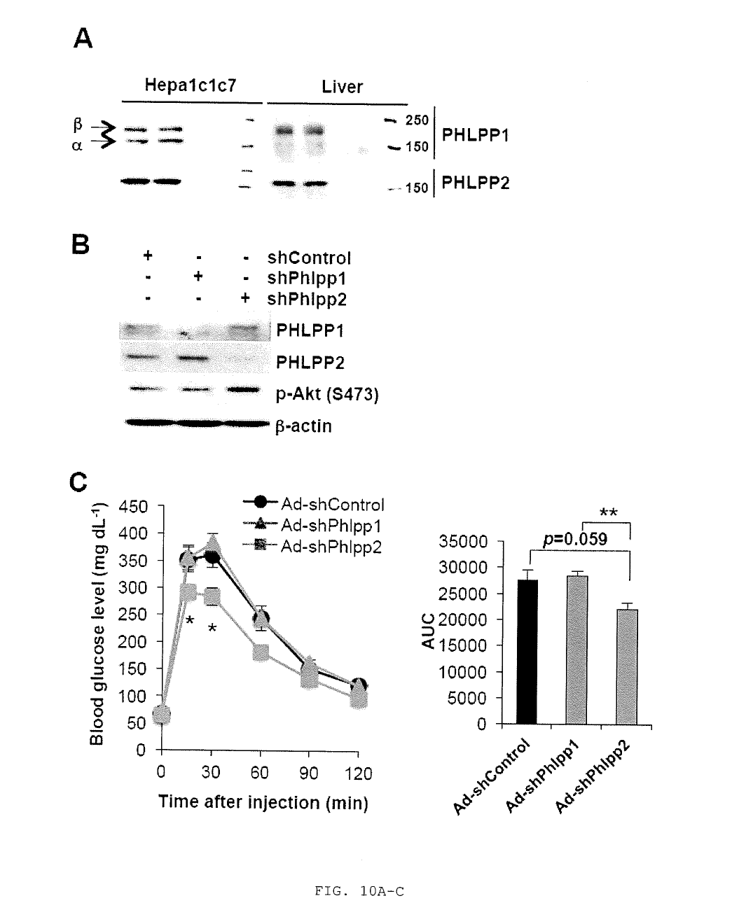

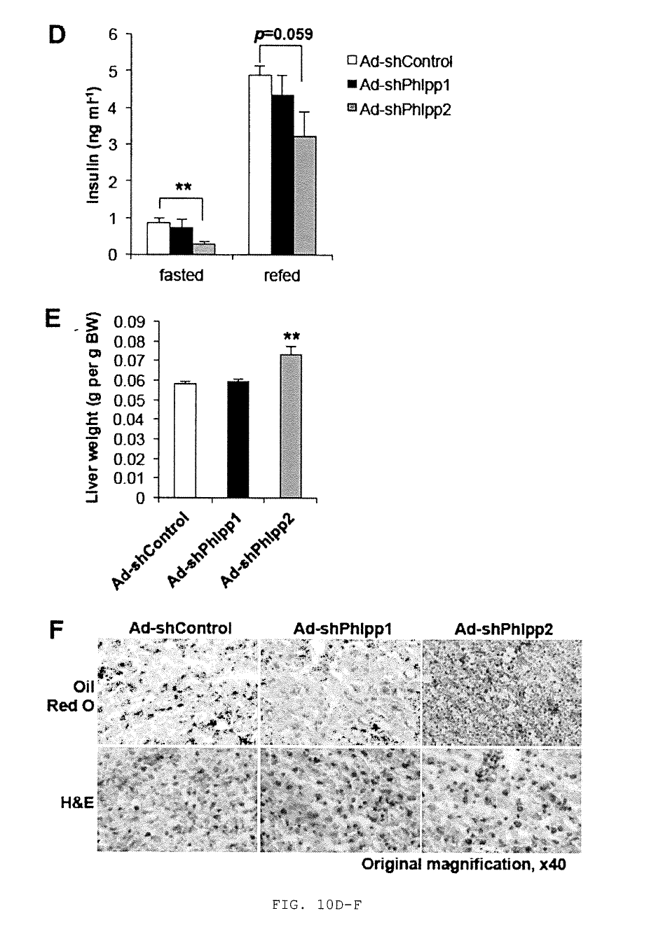

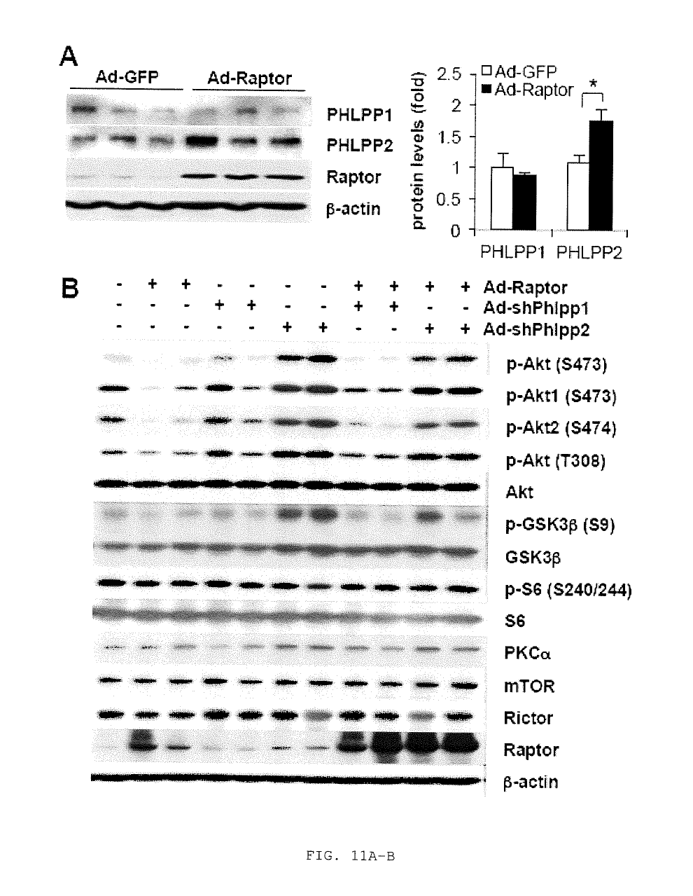

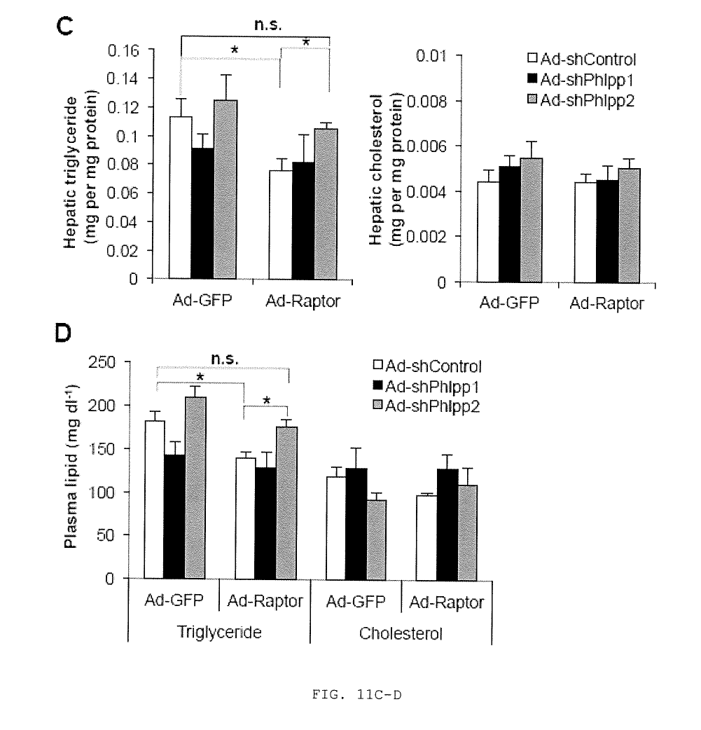

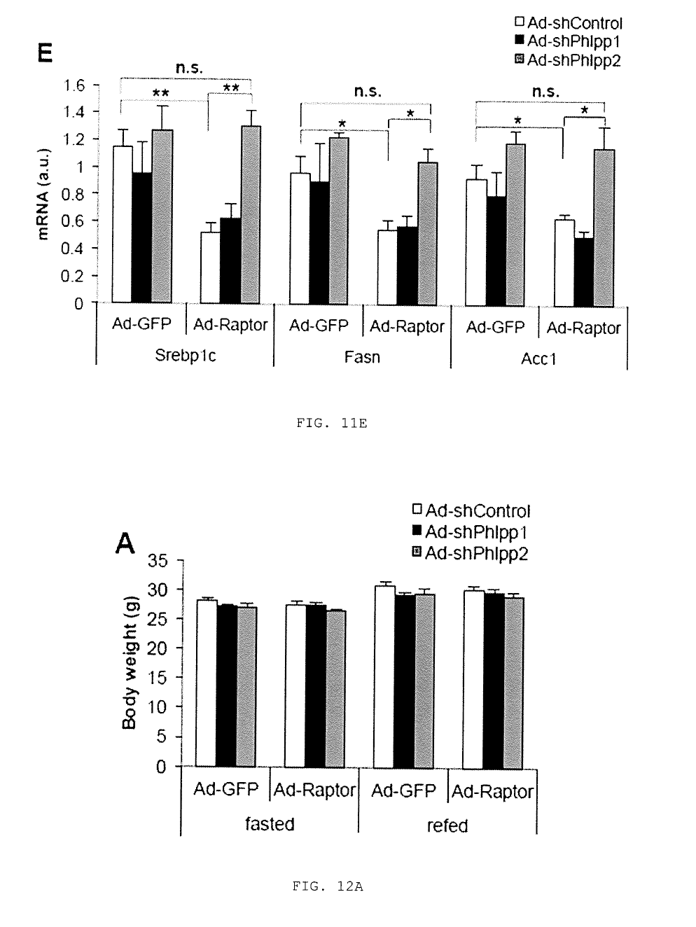

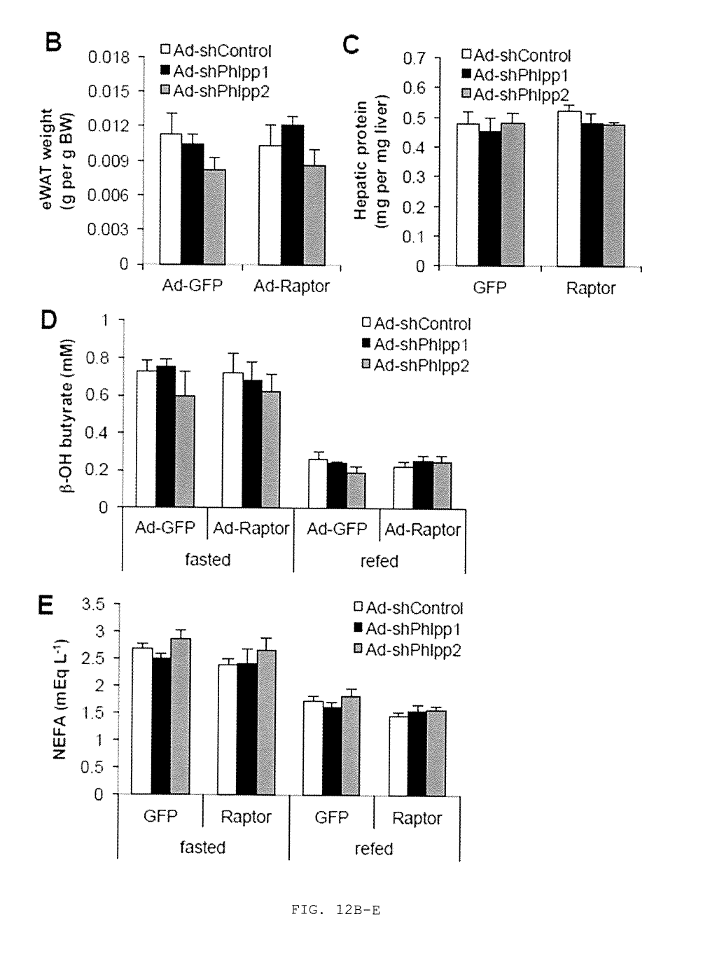

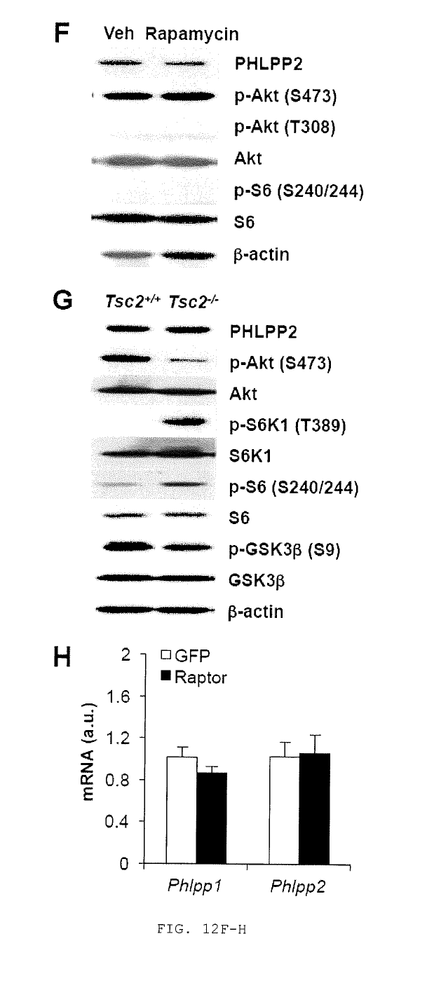

To confirm this surprising conclusion using alternative biochemical methods, a cell-permeable, non-cleavable chemical cross-linker, disuccinimidyl suberate (DSS), was used to trap native mTORC1 complexes and separate free and bound components. Similar to chromatography results (described above), while mTOR and the mTORC2-defining Rictor were predominantly found in the bound state in all mice (FIG. 2A), free Raptor was prevalent in young, chow-fed mice, but markedly decreased with aging or obesity (FIGS. 1C, 1D and 2A-2C). Again, as total levels of mTOR and Raptor did not substantially change, it was hypothesized that altered free Raptor reflected greater mTOR-Raptor association in aging or obesity. For these experiments, the cleavable cross-linker dithiobis[succinimidyl propionate] (DSP) was used to ensure complete recovery of mTORC1 components. As predicted, whereas the interaction between mTOR and GβL was consistent between lean and obese mice, mTOR-Raptor association was found to be markedly increased in ob/ob mice (FIG. 2D). Consistently, performing anti-mTOR immunoprecipitation using the zwitterionic detergent CHAPS instead of Triton X-100 to sustain the mTOR-Raptor interaction in the absence of cross-linker (Kim et al., 2002), we found greater bound Raptor in ob/ob mice (FIG. 2E).

To isolate the mechanism underlying alteration in free Raptor in obesity, a cross-linking assay was applied to hepatocytes and hepatoma cells. As it had been observed that the mTOR-Raptor complex is looser in the presence of amino acids (Kim et al., 2002), it was hypothesized that nutrient and/or hormonal signals may prompt altered Raptor-mTOR association. As previously described by the Sabatini group and others (Kim et al., 2002; Kim et al., 2003), brief amino acid deprivation increased while rapamycin treatment decreased mTORC1 recovery (FIG. 2F), but neither affected free Raptor levels (FIG. 1E). On the contrary, prolonged high-dose insulin treatment to induce insulin resistance (Cook et al., 2015) reduced free Raptor levels in primary hepatocytes or Hepa1c1c7 hepatoma cells (FIGS. 1F and 2G). Collectively, these data suggest that mTORC1 complex stability is dynamically regulated, and that the observed free Raptor deficit in steatotic liver is likely due to insulin resistance.

### EXAMPLE 2

Rescue of Free Raptor Levels Prevents Aging and Obesity-Dependent Hepatic Steatosis

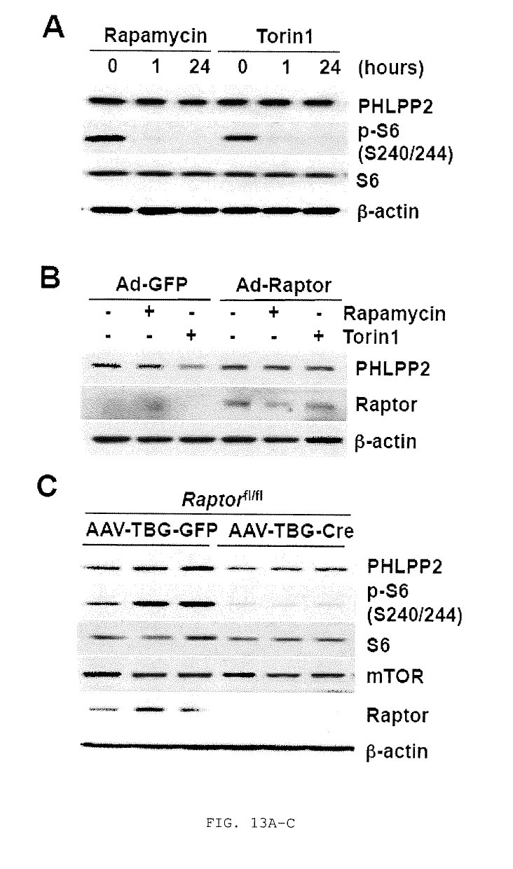

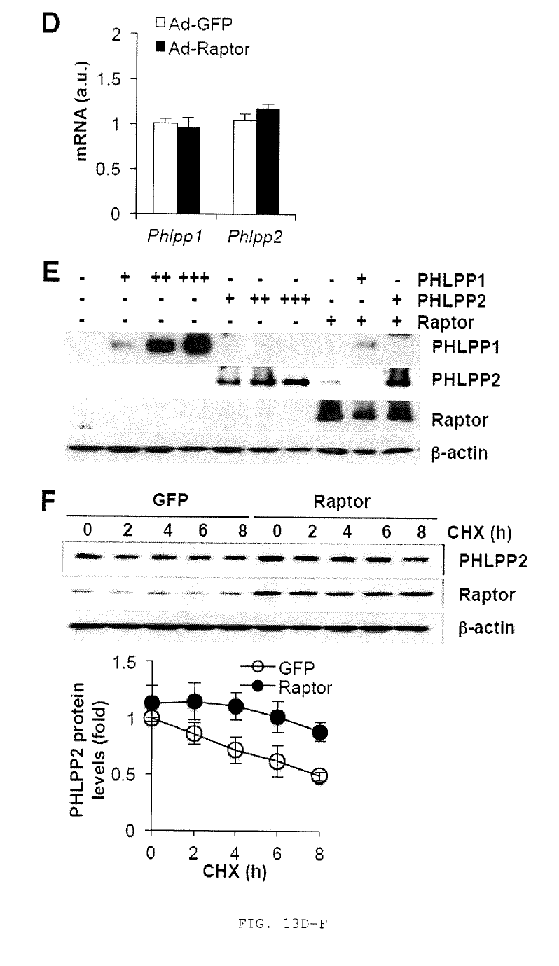

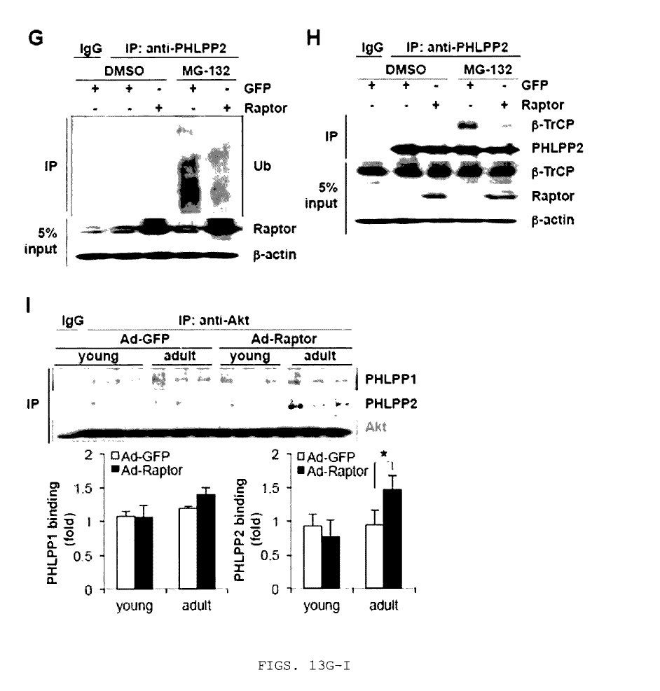

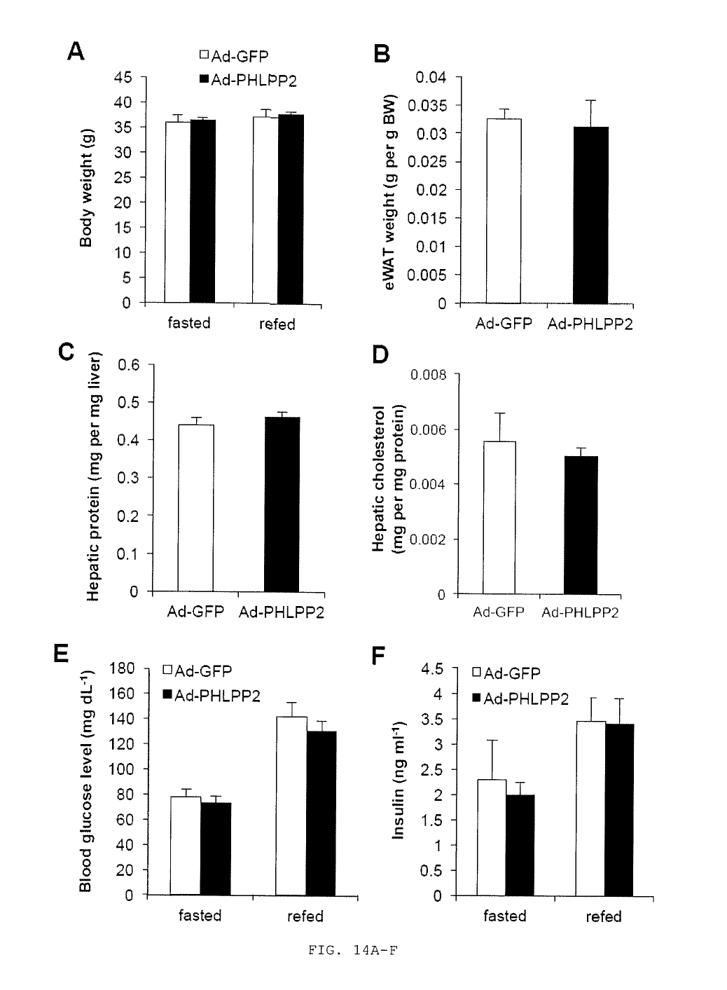

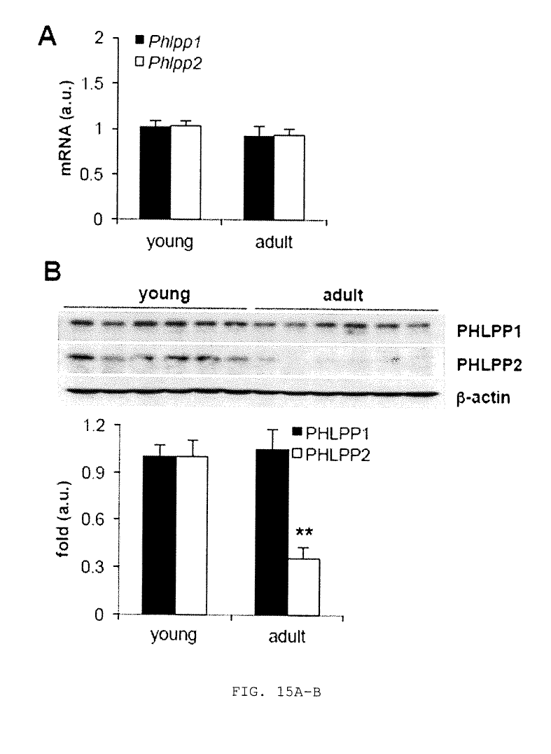

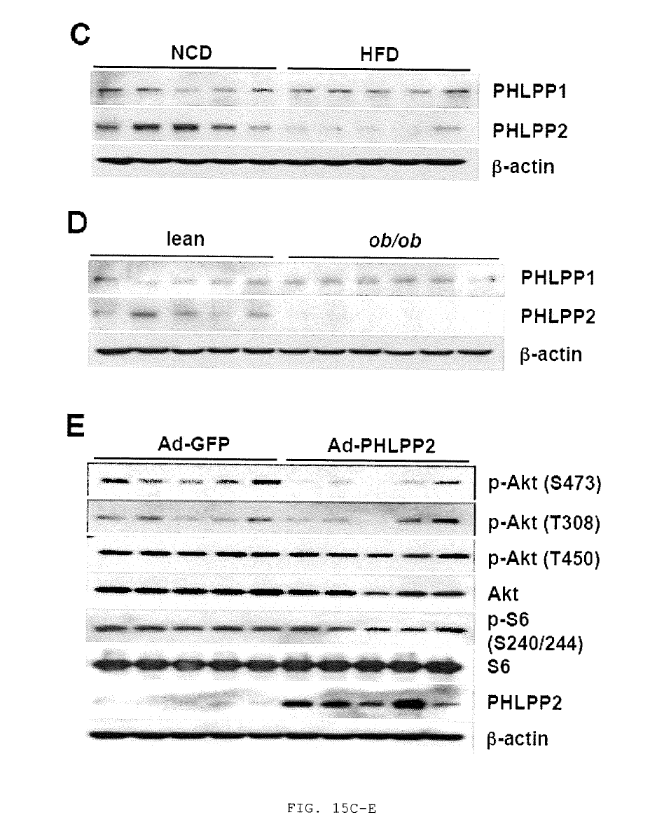

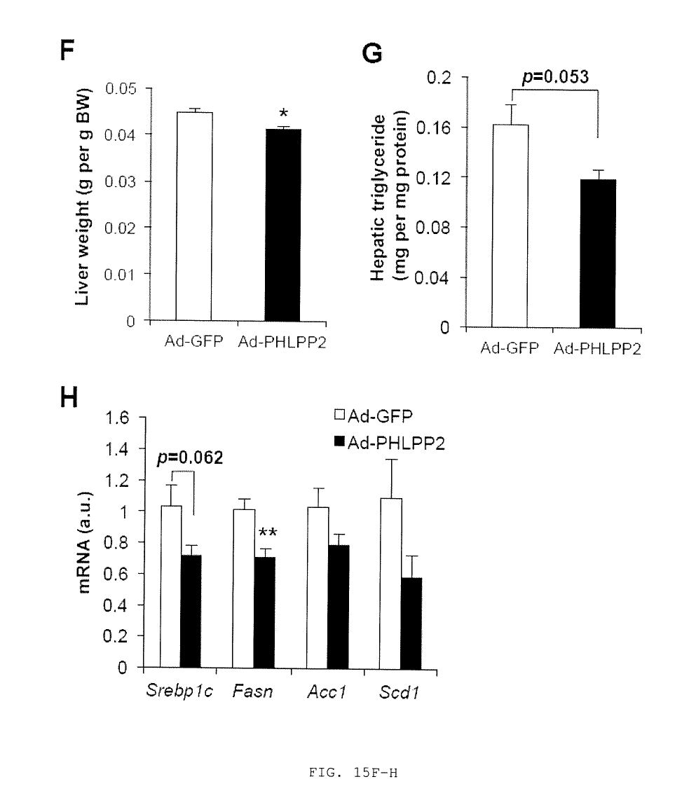

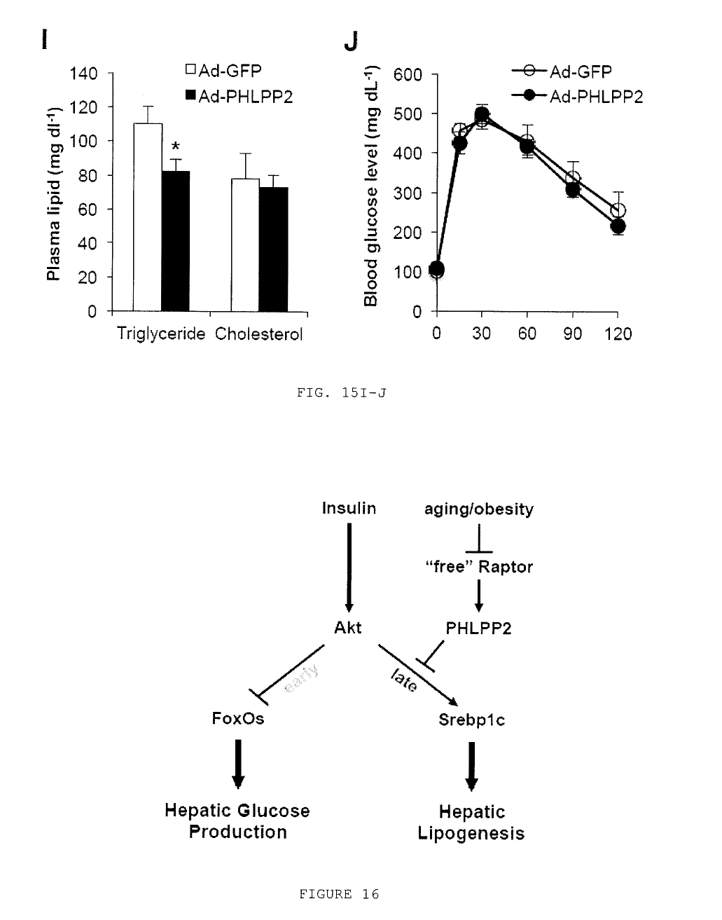

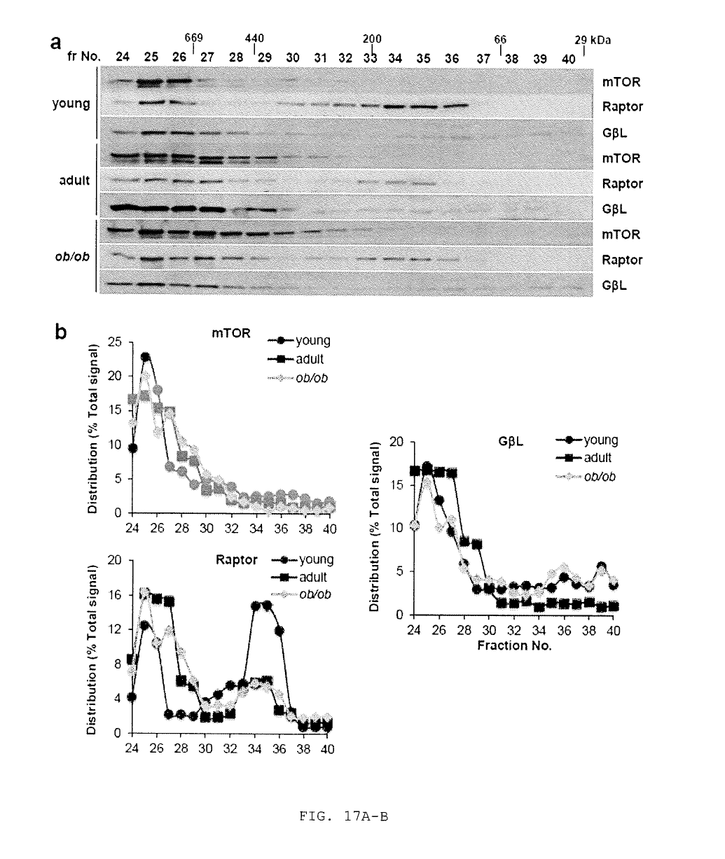

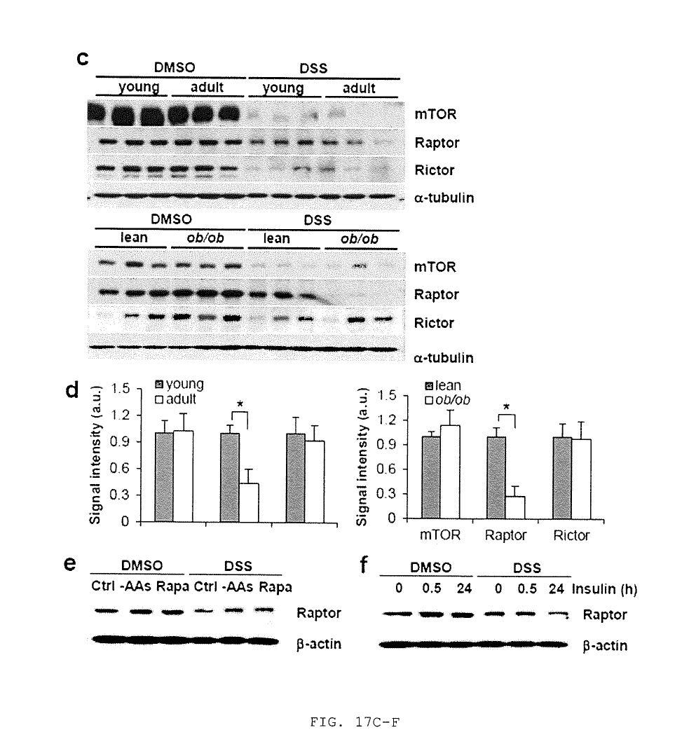

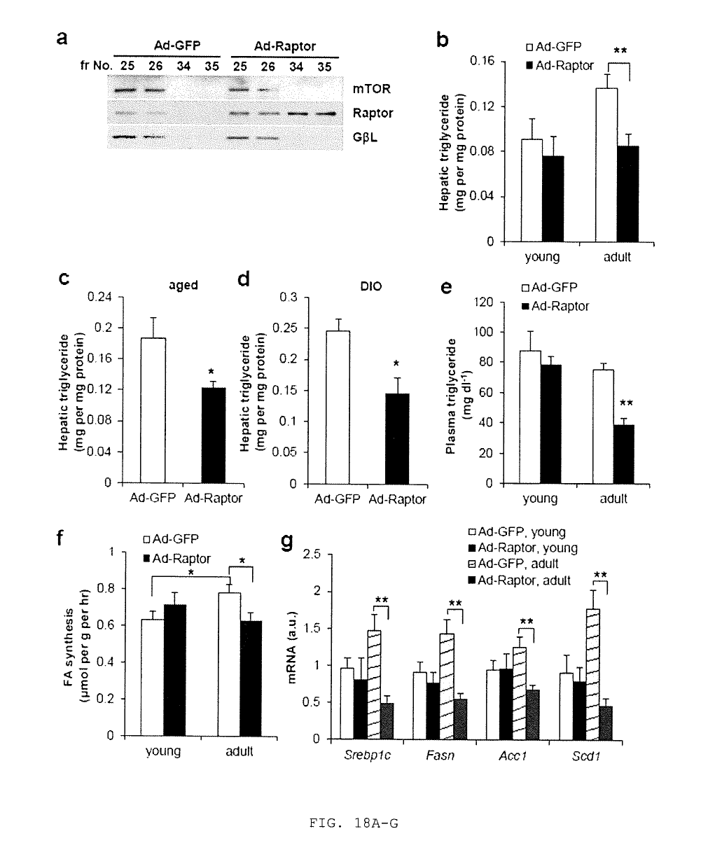

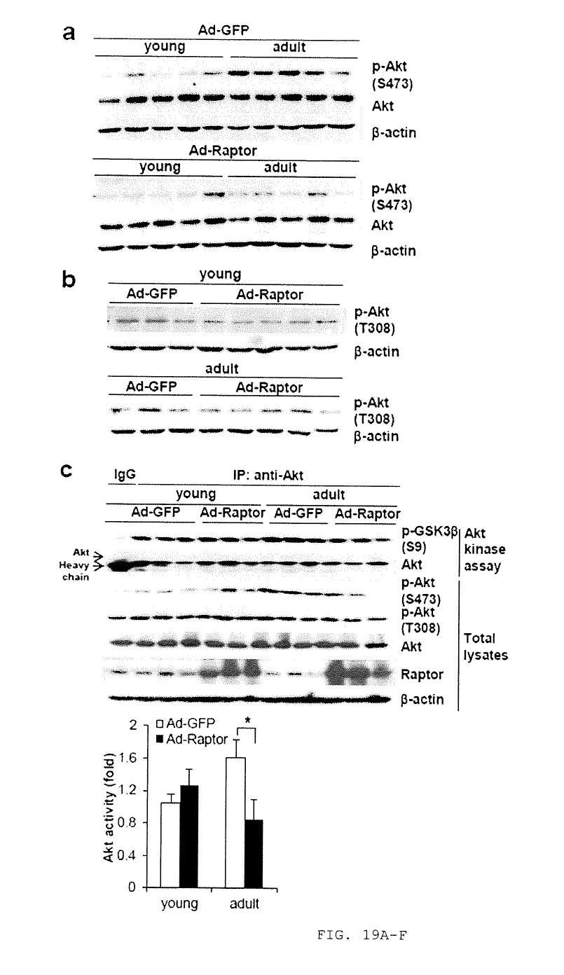

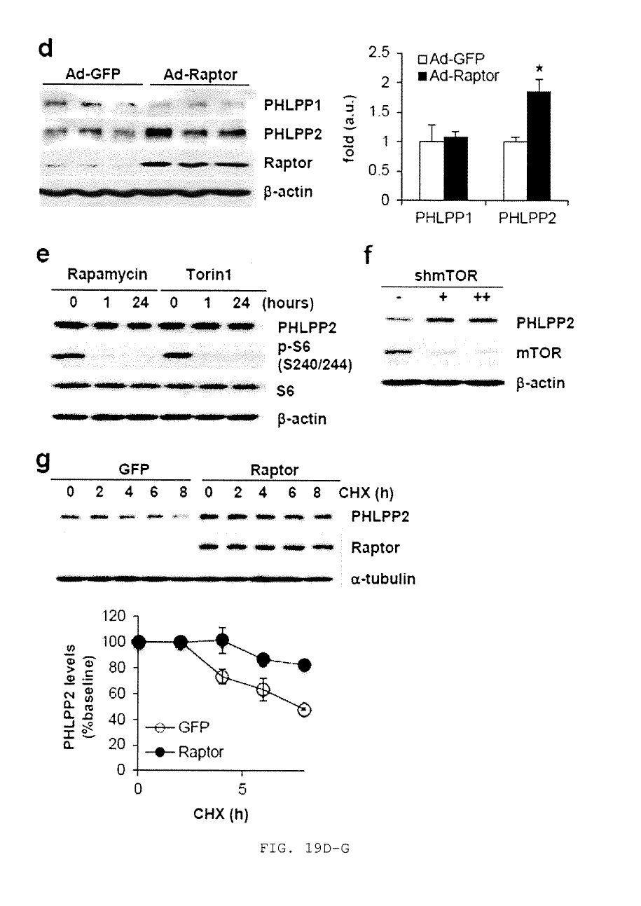

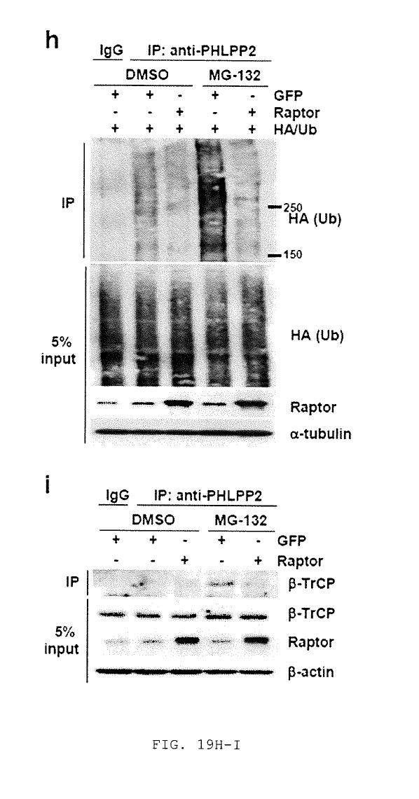

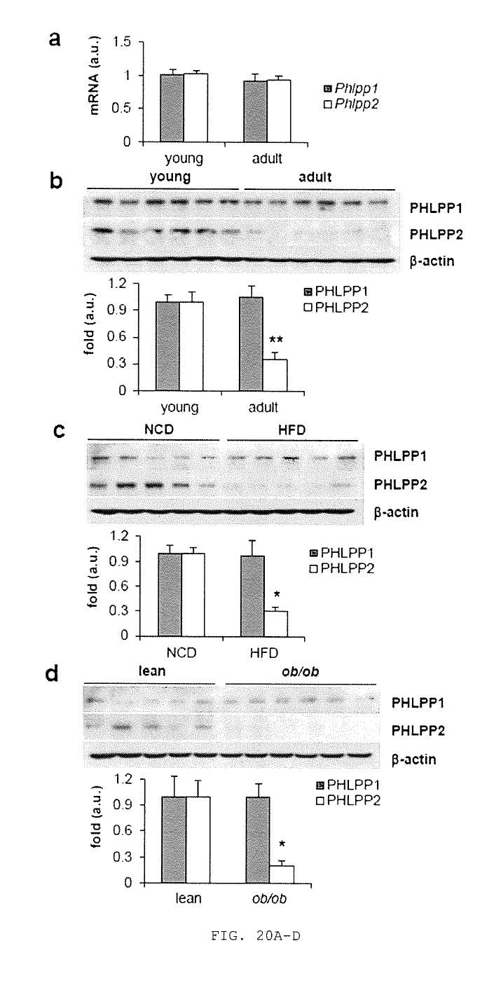

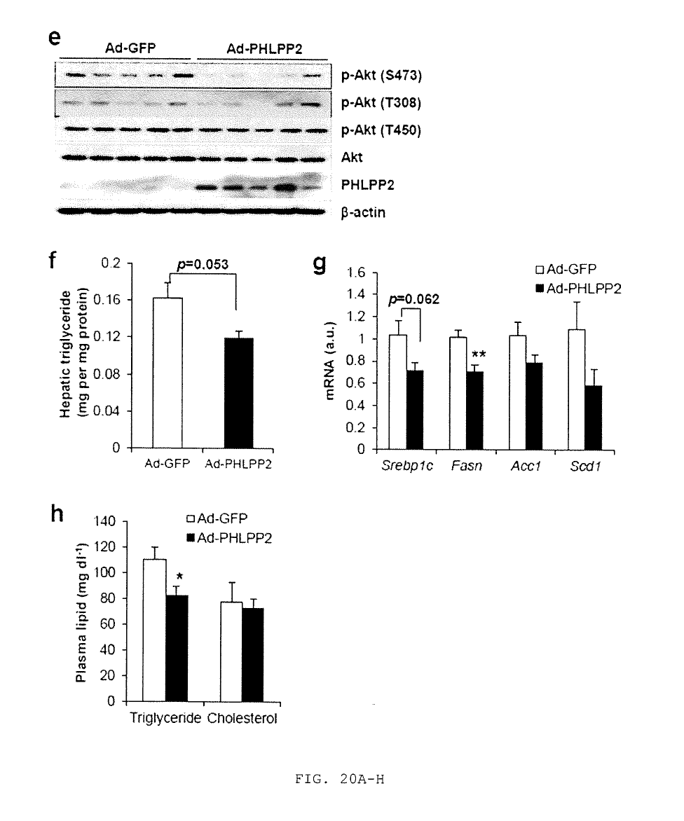

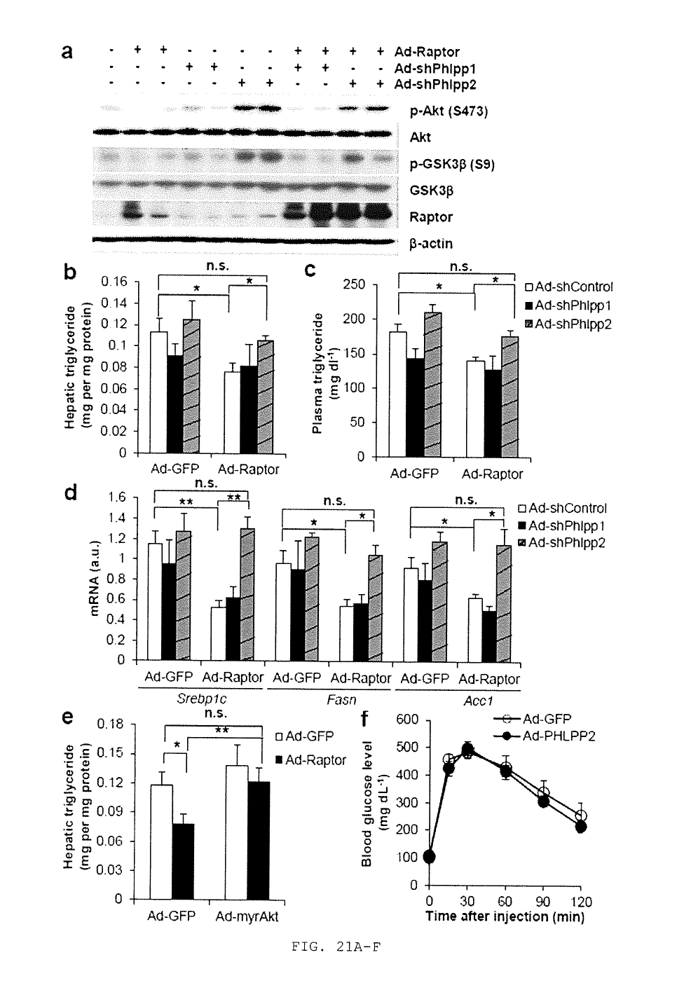

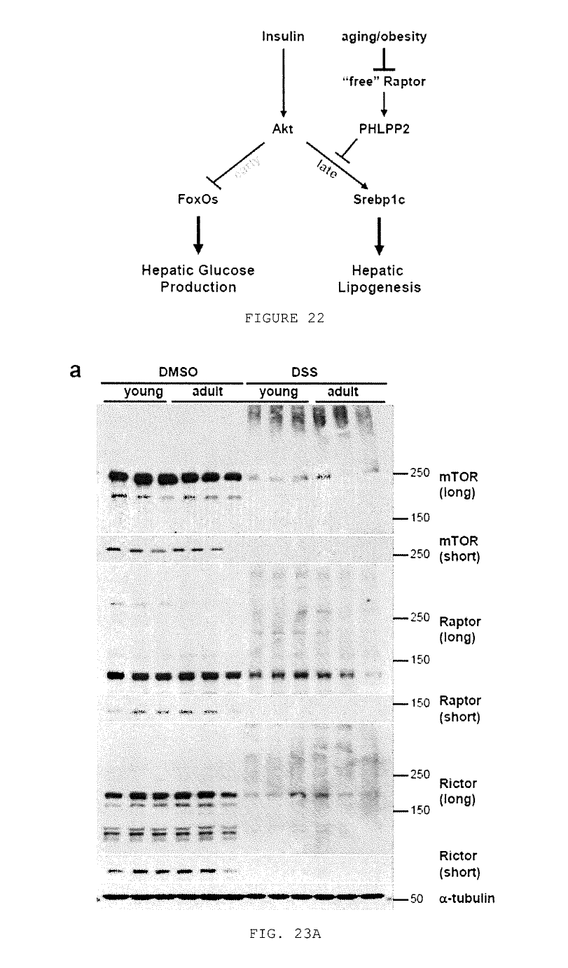

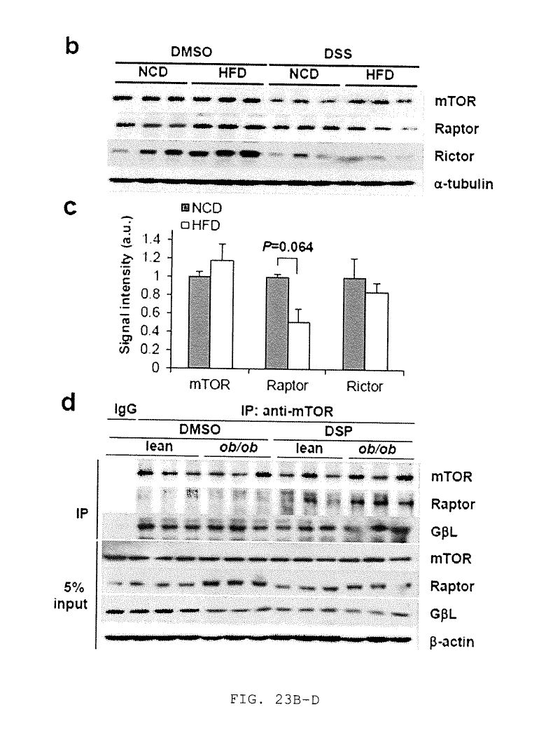

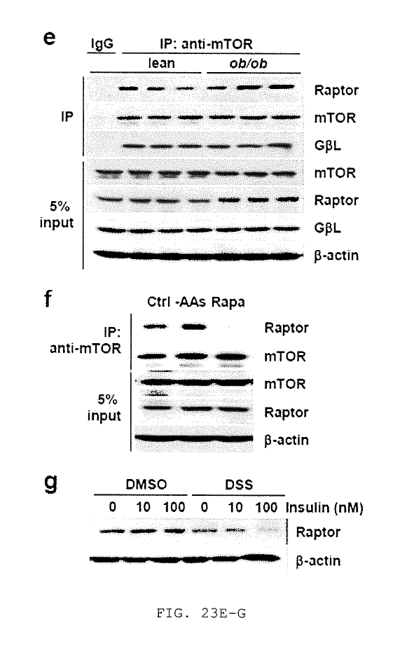

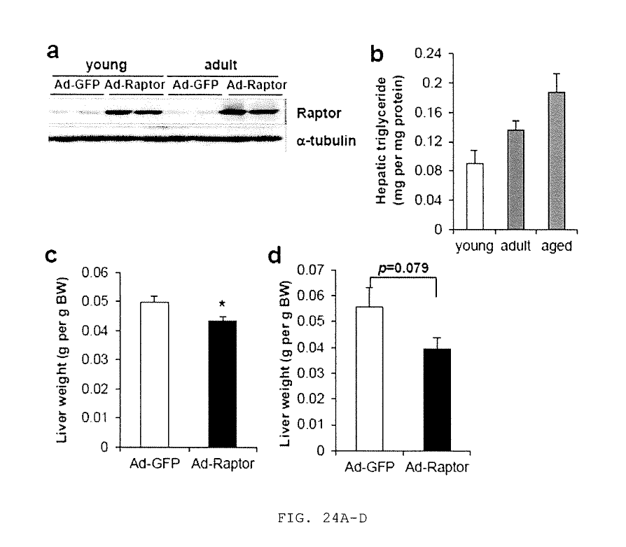

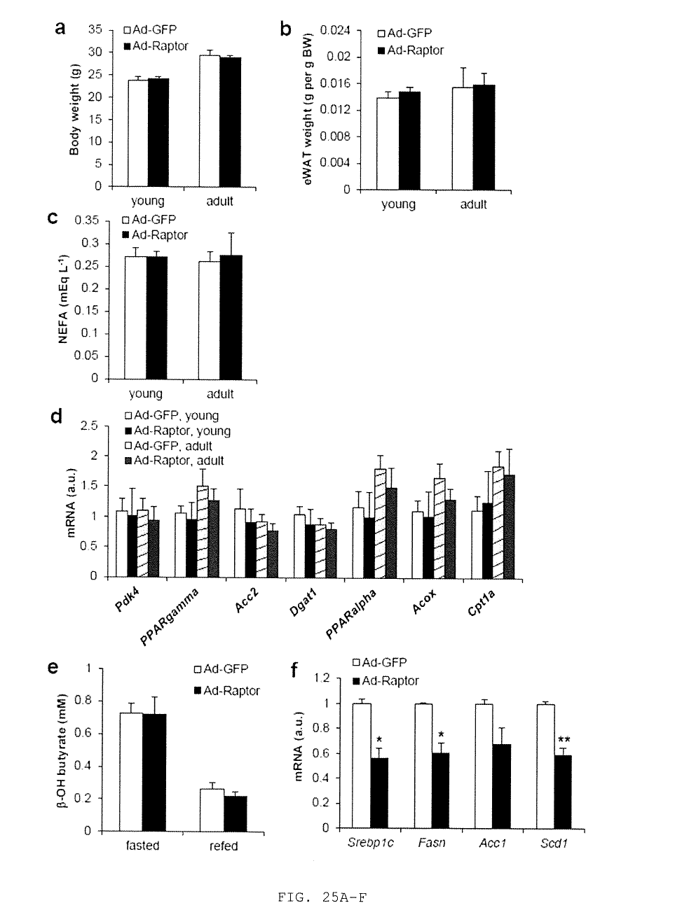

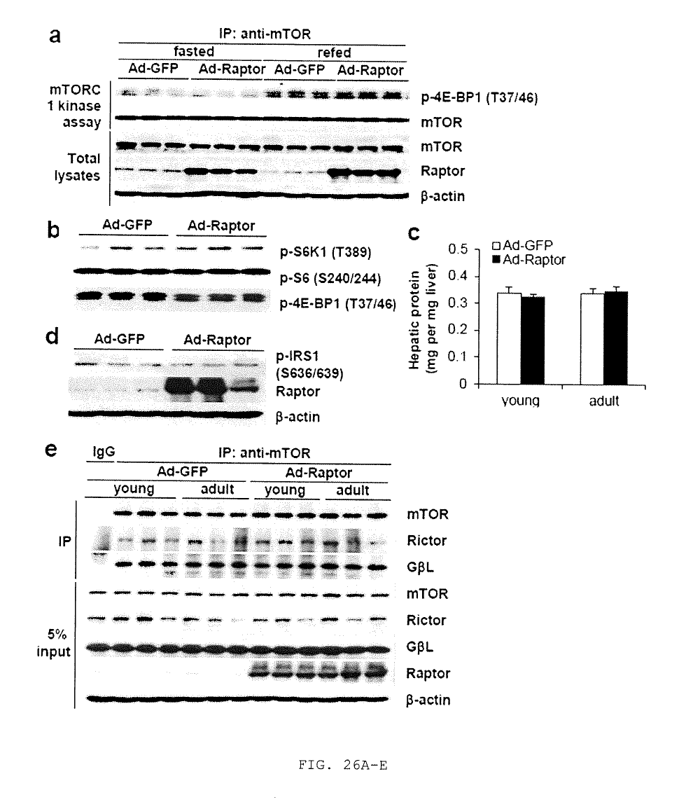

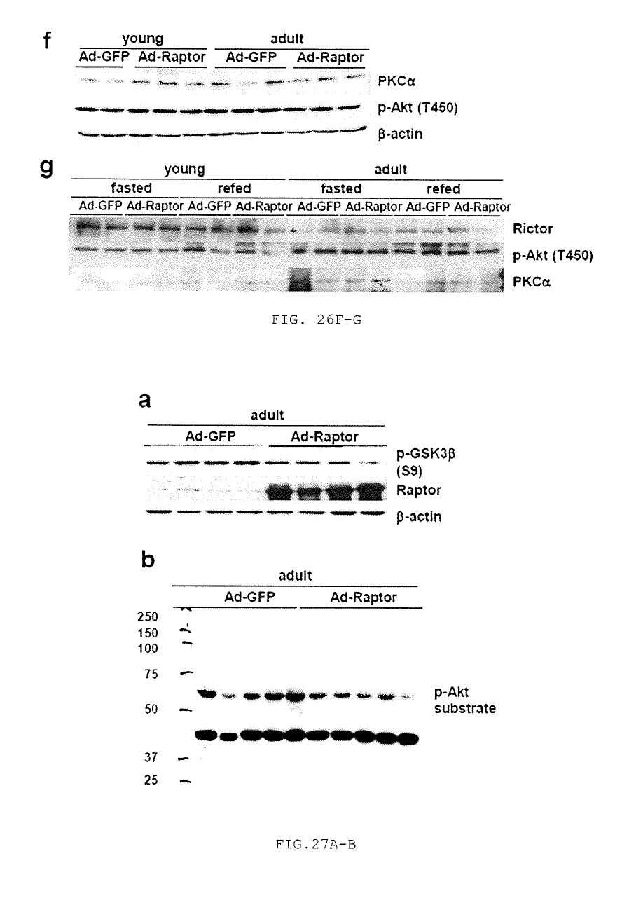

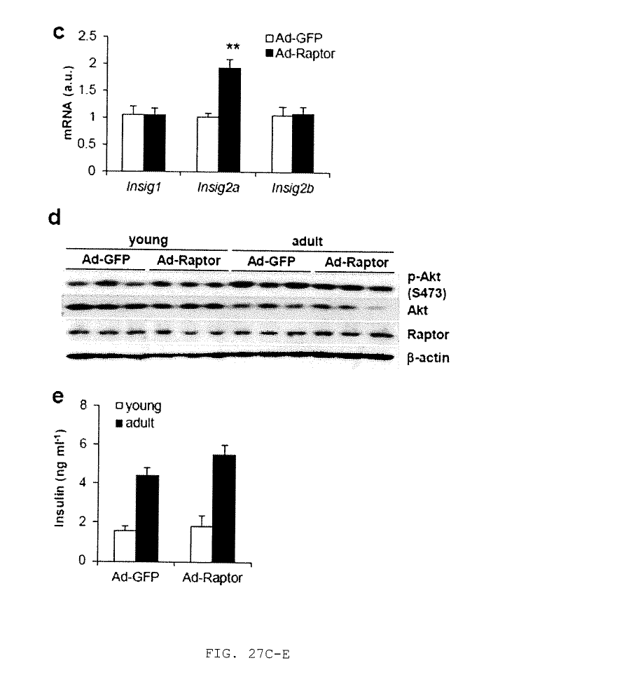

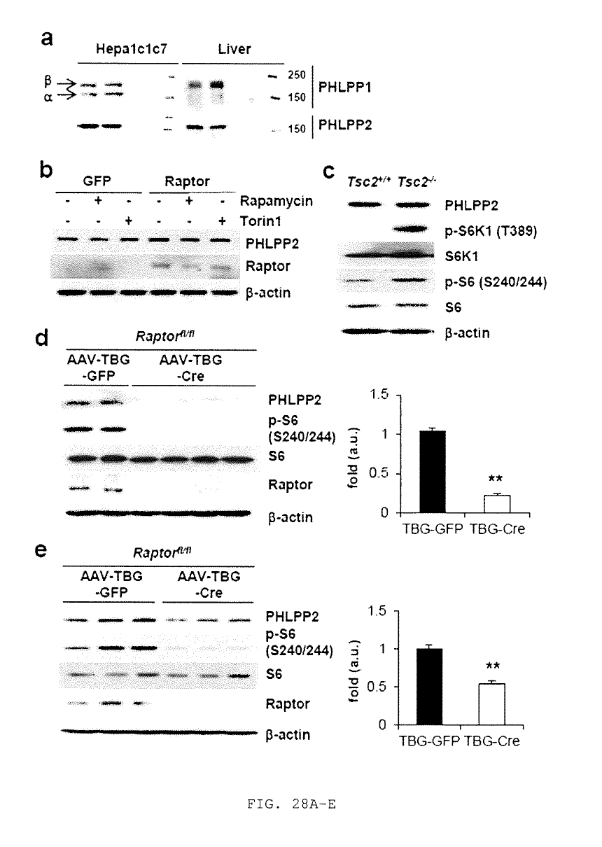

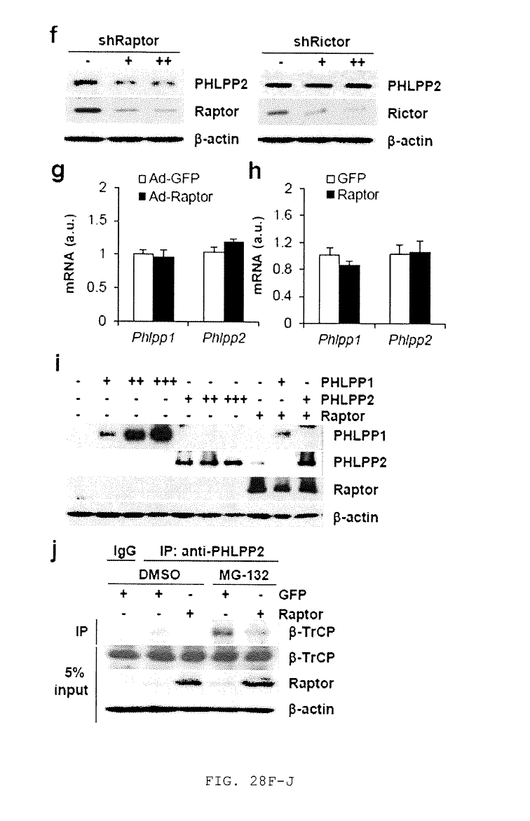

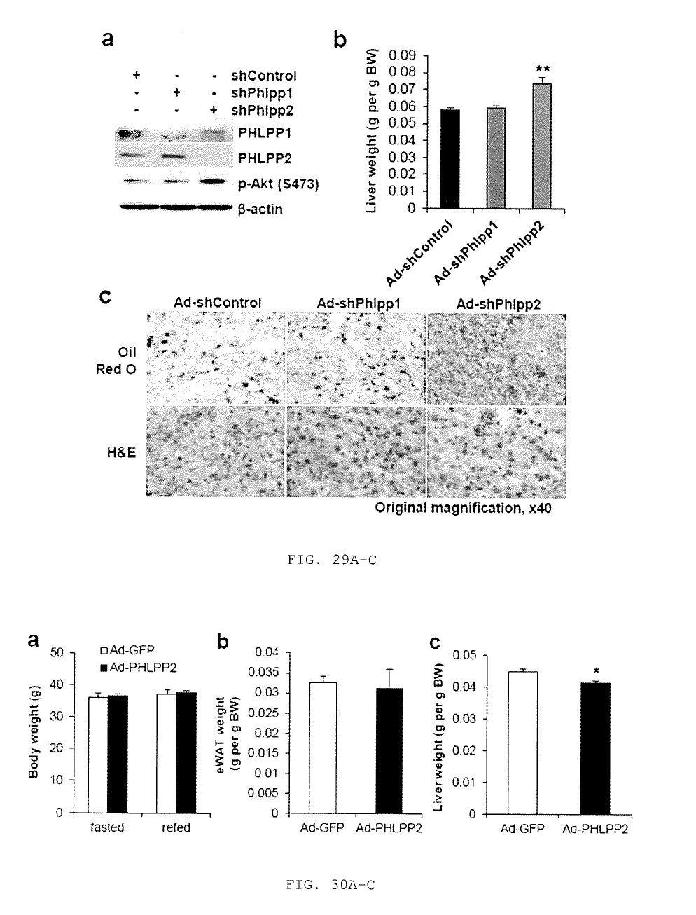

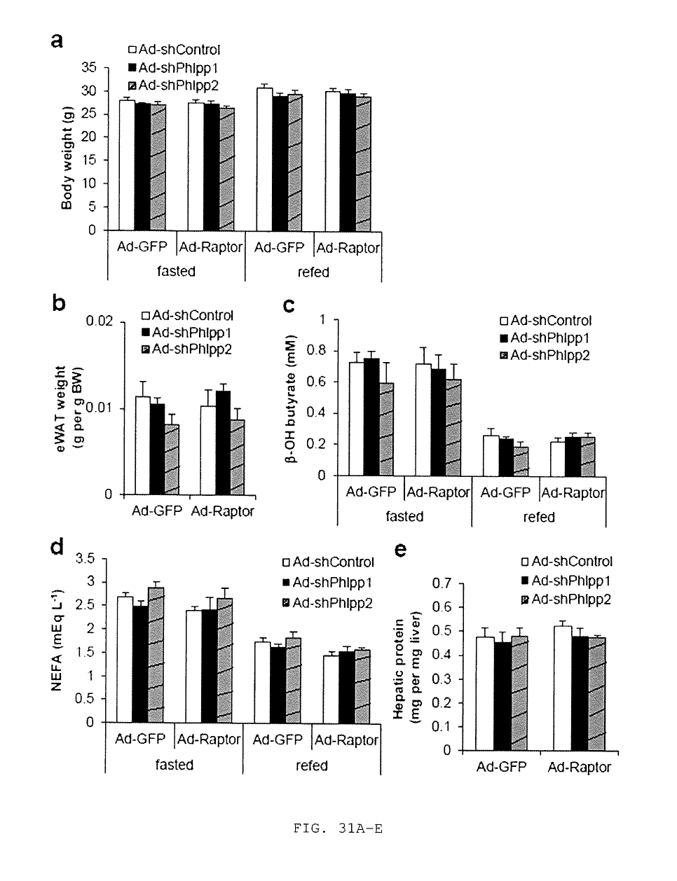

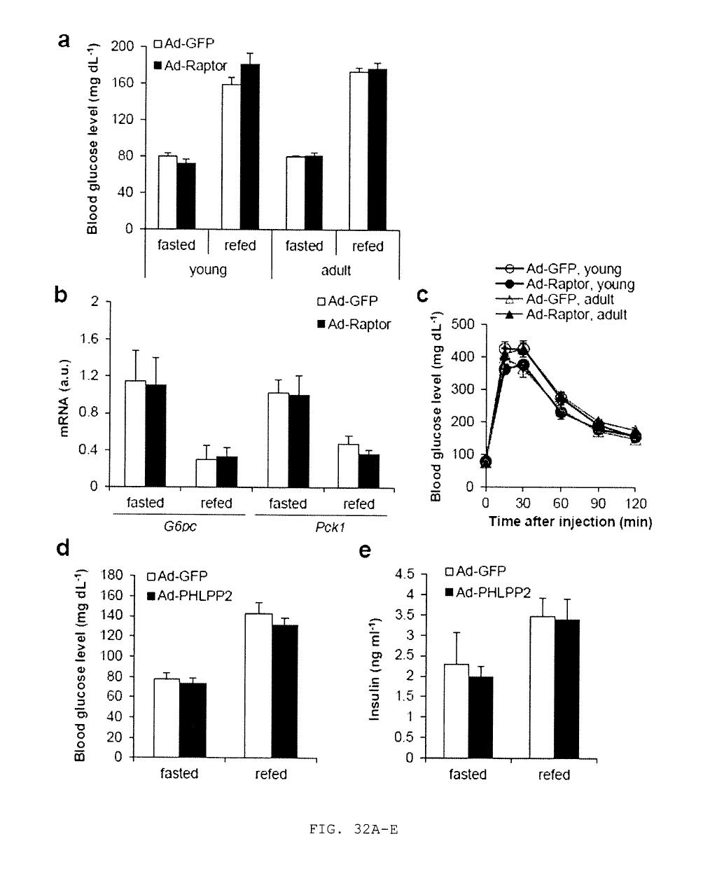

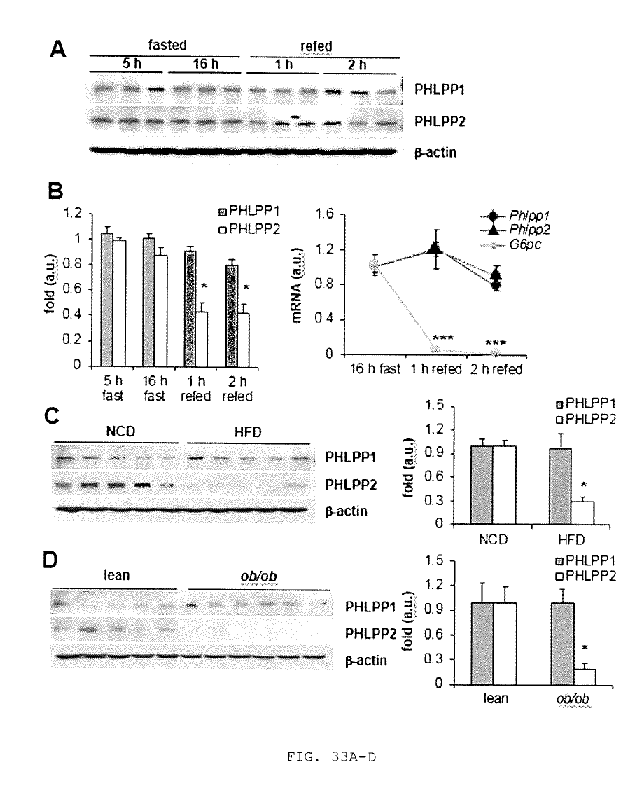

It was predicted that exogenous delivery of Raptor would increase the Raptor/mTOR ratio, and acutely, free Raptor levels. Adenoviruses expressing control (Ad-GFP) or Raptor (Ad-Raptor) were generated to induce moderate liver-specific Raptor overexpression (FIG. 3A). As predicted, the majority of overexpressed Raptor eluted at ˜150 kDa, distinct from mTOR-containing fractions (FIG. 3B). Next, whether increasing free Raptor levels would prevent hepatic steatosis associated with insulin resistance induced by two different pathophysiologic conditions—aging and obesity were tested. First, aged (10-12 month-old), wild-type mice were transduced with Ad-GFP or Ad-Raptor. No difference in body weight or adiposity was found (FIGS. 3C and 3D), but observed that livers from Ad-Raptor mice were smaller (FIG. 3E) and less pale in color (data not shown). As predicted from the gross anatomic appearance, Ad-Raptor mice showed lower (˜34%) hepatic TG as compared to control mice, without change in hepatic cholesterol (FIG. 4A). Next, the experiment was repeated in diet-induced obese (DIO) mice and again a weight- and adiposity-independent reduction in liver weight was observed (FIGS. 3F-3H) and hepatic TG (FIG. 4B) in Ad-Raptor mice.

These data show that hepatic Raptor overexpression reduces aging and obesity-related hepatic TG accumulation. It was hypothesized that the lipid-sparing effect may be specific to aged or overfed mice, and repeated these experiments in both young (8-week-old) and adult (24-week-old) chow-fed animals. As expected, hepatic TG content was found to be increased incrementally from young (0.09 mg/mg protein) to adult (0.14 mg/mg protein) to aged (0.19 mg/mg protein) (FIG. 5A). Ad-Raptor reduced liver weight (FIG. 5B) and TG content (FIG. 4C, FIG. 63D) in adult mice, to a surprisingly similar degree as in aged or obese mice (˜10% reduction in liver weight, ˜35% reduction in liver TG), but did not affect either parameter in young animals despite a similar increase in Raptor levels (FIG. 5C). Collectively, these data indicate that free Raptor proportionately prevents hepatic TG accumulation, even relatively early in the morbid state. As such, to avoid potential confounding induced by advanced age and/or obesity, the remainder of the studies were restricted to a comparison of young and adult mice.

### EXAMPLE 3

Increasing Free Raptor Levels Reduces Fatty Acid Synthesis

Given the known hepatocyte tropism of adenovirus (Wang et al., 2001), it was hypothesized that Raptor overexpression reduced hepatic TG in a cell-autonomous manner. Indeed, no difference were observed in food intake, body weight, non-esterified fatty acid (NEFA) or plasma cholesterol levels (not shown and FIGS. 5D-5F), although plasma TG was lowered in parallel with hepatic TG in both aged (FIG. 4D) and adult (FIG. 4E) Ad-Raptor mice. Next, 3H2O was injected into Ad-Raptor and control mice, and incorporation of label was measured into newly synthesized hepatic fatty acid by de novo lipogenesis (DNL).

Expectedly, aging was associated with increased DNL (FIG. 4F, FIG. 63C), and a trend towards higher expression of key lipogenic genes, Srebp1c, Fasn (fatty acid synthase), Acc1 (acetyl-CoA carboxylase 1), and Scd1 (steroyl-CoA desaturase 1) (FIG. 4G, FIG. 63B), all of which was reversed by increased free Raptor. Expression of fatty acid oxidation and other lipogenic genes were not significantly altered (FIG. 5G). Raptor overexpression in primary hepatocytes isolated from adult mice led to similar reduction in lipogenic gene expression (FIG. 5H), confirming that this effect is cell-autonomous. In sum, this data suggest that rescue of aging-dependent free Raptor deficit reduces Srebp1c-dependent DNL, leading to less hepatic and plasma TG.

### EXAMPLE 4

Increase in Free Raptor Levels Does Not Affect mTORC1 or mTORC2 Activity

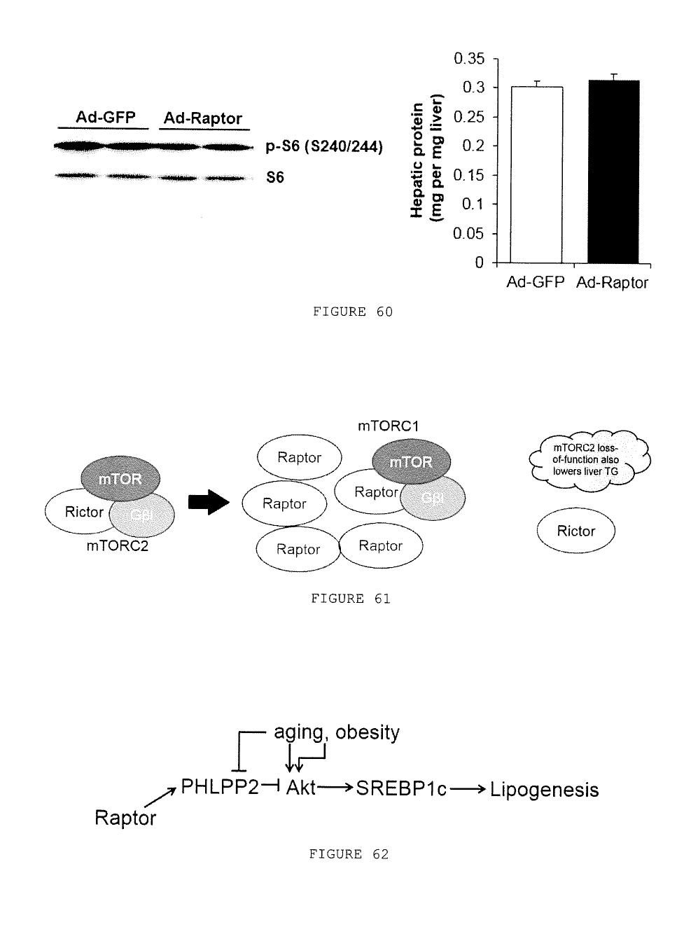

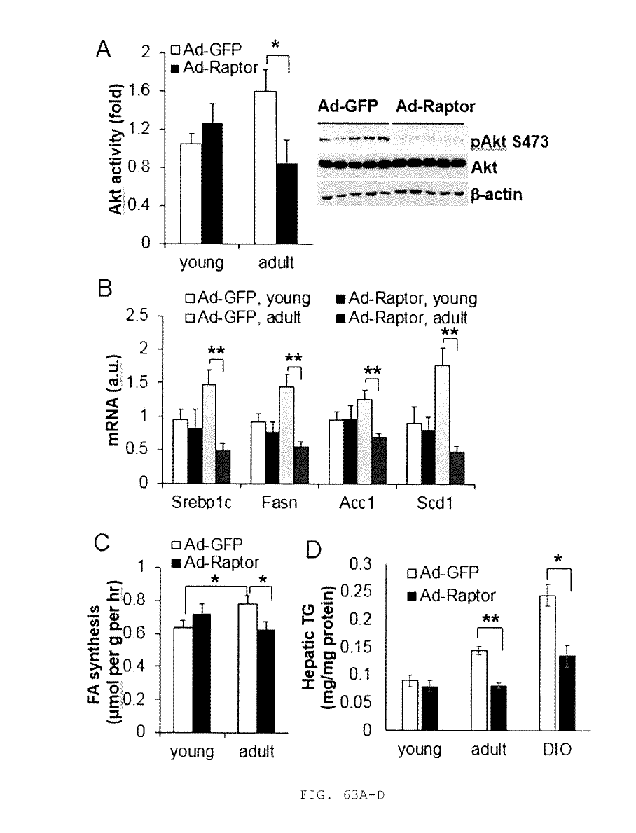

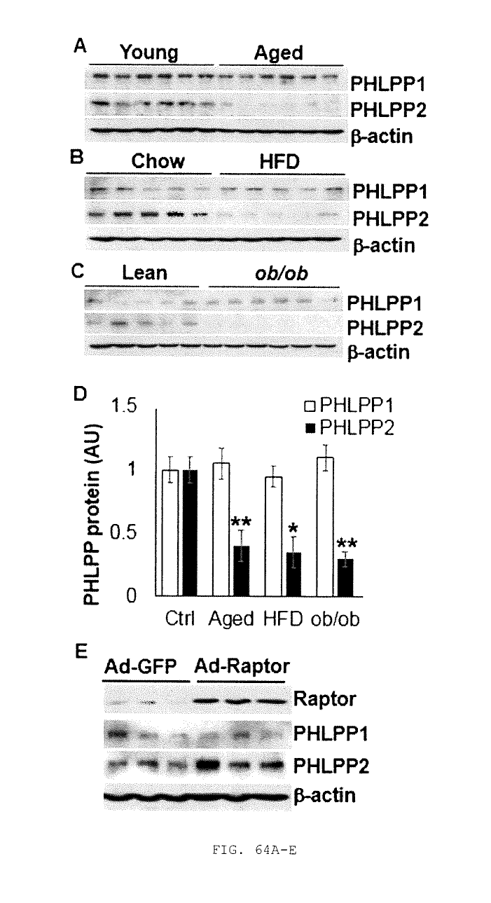

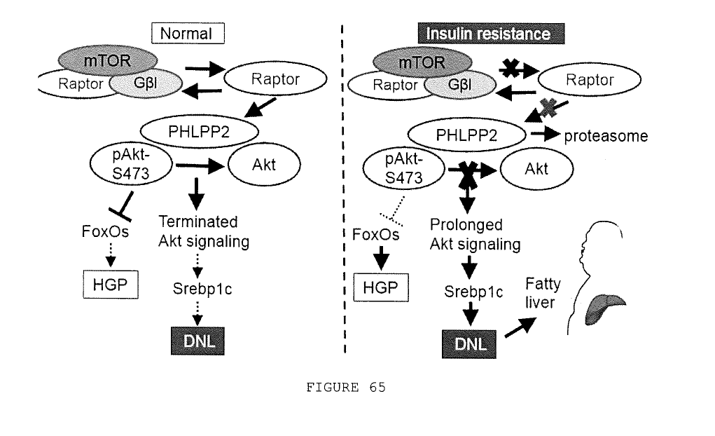

As mTORC1 activity is thought to increase with aging (Hands et al., 2009), and genetic or pharmacologic inhibition of mTORC1 reduces Srebp1c activity (Li et al., 2010; Peterson et al., 2011), it was hypothesized that the specificity of Ad-Raptor to prevent steatosis in older or obese mice was due to reduced mTORC1 function. This would be consistent with in vitro data suggesting that Raptor overexpression can decrease mTORC1 kinase activity (Kim et al., 2002). Surprisingly, phosphorylation of canonical mTORC1 targets was found to be unchanged (FIGS. 6A and 6B) and normal hepatic protein content (FIG. 6C) in young, adult or aged Ad-Raptor mice as compared to controls, in stark contrast with the 40% reduction in hepatic protein content and lower mTORC1 target phosphorylation observed in L-Raptor mice (Sengupta et al., 2010). Next, an alternative hypothesis was tested whereby the addition of excess Raptor could “steal” shared mTOR components, leading to reduced mTORC2 activity and recapitulate the lipid-sparing phenotype of L-Rictor mice (Hagiwara et al., 2012). mTORC2 has been shown to phosphorylate the turn (Thr450) motifs in Akt and PKCα to regulate stability of these proteins (Facchinetti et al., 2008; Ikenoue et al., 2008). Ad-Raptor mice showed normal p-Akt Thr450 and PKCα levels (FIGS. 6D and 6E), commensurate with normal mTOR-Rictor interaction in Ad-Raptor liver (FIG. 6F). In sum, these data suggest that altered mTORC1 or mTORC2 complex stability and activity is unlikely to explain free Raptor-mediated reduction in hepatic TG.

### EXAMPLE 5

Free Raptor Decreases Akt Ser473 Phosphorylation and Activity

In parallel with the above results, it was noted that refeeding-induced phosphorylation of Akt at Ser473, another known mTORC2 substrate (Sarbassov et al., 2005), was increased by aging in Ad-GFP, but not Ad-Raptor mice (FIG. 7A). We repeated the experiment in a separate cohort, and again observed abrogation of aging-induced refed Akt Ser473 phosphorylation in Ad-Raptor mice (FIG. 8A), without significant changes in plasma insulin levels (FIG. 7B) or PI3K/PDK1-mediated phosphorylation at Thr308 (FIG. 7C). Reduced Akt Ser473 phosphorylation was not observed in extra-hepatic tissues (FIG. 8B), suggesting a cell-autonomous effect of Ad-Raptor as opposed to a whole-body change in insulin sensitivity. As both Thr308 and Ser473 phosphorylation events are important for full activation of Akt (Bhaskar and Hay, 2007), it was next examined whether Raptor overexpression reduces Akt activity. Indeed, we observed decreased phosphorylation of GSK3β at Ser9 (FIGS. 8A and 8C) and general Akt substrate phosphorylation (FIG. 8D) in livers of refed Ad-Raptor mice, as well as reduced immunoprecipitated Akt kinase activity on recombinant GSK3β (FIG. 7D, FIG. 63A). Similarly, Raptor overexpression in primary hepatocytes was accompanied by diminished basal and insulin-stimulated Akt Ser473 phosphorylation (FIG. 8E). Interestingly, plasma glucose (FIG. 8F), gluconeogenic gene expression (FIG. 8G), and glucose tolerance (FIG. 7E) were unchanged in Ad-Raptor mice, suggesting that significant reductions in Akt activity can be tolerated without altered glucose homeostasis. These data demonstrate a selective regulation of Akt's lipogenic action by free Raptor.

### EXAMPLE 6

Rescue of Akt Activity Increases Hepatic TG in Ad-Raptor Mice

Given the known role of Akt in inducing DNL and fatty liver (Li et al., 2010), it was hypothesized that defective hepatic Akt activation by free Raptor caused the observed reduced hepatic TG. To prove this, Ad-Raptor (or control) mice were co-transduced with either Ad-GFP or constitutively active, myristoylated Akt (Ad-myrAkt). A low dose of myrAkt was intentionally chosen as higher doses of this potent activator promotes hepatic steatosis (Calvisi et al., 2011; Han et al., 2009; Li et al., 2013). As hypothesized, free Raptor protection from fatty liver was abrogated by co-transduction with Ad-myrAkt (FIG. 7F), without change in body or adipose weight, blood glucose or hepatic protein concentration (FIGS. 9A-9D). Taken together, these data indicate that free Raptor in liver maintains normal hepatic TG by restricting excessive or prolonged Akt activation.

### EXAMPLE 7

Raptor Increases PHLPP2 Levels to Terminate Akt Signaling and Reduce Liver TG

Insulin levels and mTORC2 activity were unaffected by hepatic Raptor overexpression. As such, it was hypothesized that termination of the Akt signal, as opposed to decreased activation by insulin/PDK1 and mTORC2, is increased in Ad-Raptor mice. This is consistent with the finding that even myrAkt activity on endogenous GSK3β was mildly attenuated by free Raptor (FIG. 7G). PHLPPs are recently described phosphatases that terminate Akt signaling by dephosphorylation of Ser473; in cancer lines, PHLPPs have been shown to suppress tumor growth via this action (Gao et al., 2005). The PHLPP family is encoded by two different genes—PHLPP1, with two splice variants (PHLPP1α and PHLPP1β) and PHLPP2 (Brognard et al., 2007; Gao et al., 2005). To test the hypothesis free Raptor may alter PHLPP activity, isoform distribution in liver was first surveyed. PHLPP1β and PHLPP2 (but not PHLPP1α) were found to be expressed (FIG. 10A), and that Raptor overexpression selectively increased hepatic PHLPP2 levels (FIG. 11A).

These data suggest that PHLPP2 may regulate hepatic Akt activity, similar to its role in cancer. To test this, adenoviruses were contructed encoding shRNA targeting either Phlpp1 (Ad-shPHLPP1) or Phlpp2 (Ad-shPHLPP2). In primary hepatocytes, knockdown of PHLPP2 but not PHLPP1 increased Akt Ser473 phosphorylation (FIG. 10B). Similar data was obtained with 2 other independent targeting sequences (data not shown). Correspondingly, mice transduced with Ad-shPHLPP2 were more glucose tolerant with lower plasma insulin levels than control or Ad-shPHLPP1-transduced mice (FIGS. 10C and 10D). Upon sacrifice, livers from Ad-shPHLPP2 mice were larger and steatotic (FIGS. 10E and 10F). This phenotype mirrors that of mice transduced with high-dose myr-Akt (Calvisi et al., 2011; Li et al., 2013), and suggests that PHLPP2 is necessary to terminate endogenous liver Akt activity. These results allowed testing whether reduced Akt activity conferred by free Raptor is PHLPP2-dependent. Ad-Raptor (or Ad-GFP control) mice were co-transduced with Ad-shControl, Ad-shPHLPP1 or Ad-shPHLPP2 adenoviruses. PHLPP2 knockdown restored Akt Ser473 phosphorylation in Ad-Raptor mice (FIG. 11B), resulting in hepatic and plasma TG levels comparable to control mice (FIGS. 11C and 11D), without altering body weight/adiposity, hepatic protein concentration, β-hydroxybutyrate or NEFA levels (FIGS. 12A-12E). Similarly, Ad-Raptor-reduced lipogenic gene expression was corrected with PHLPP2 knockdown (FIG. 11E), without affecting canonical mTORC1/2 signaling (FIG. 11B). It was concluded that induction of PHLPP2 by free Raptor is necessary to reduce DNL and consequent hepatic steatosis.

### EXAMPLE 8

Free Raptor Blocks PHLPP2 Degradation and Increases its Interaction with Akt

Next, attention was turned to the mechanism by which free Raptor increases PHLPP2, which based on earlier results, was predicted to be independent of mTOR catalytic activity. Indeed, neither short-term amino acid deprivation (not shown), nor short- or long-term treatment with two different mTOR inhibitors, rapamycin or Torin1, decreased basal (FIGS. 13A and 12F) or Raptor-increased PHLPP2 levels (FIG. 13B). PHLPP2 levels were also unaffected by increased mTORC1 activity in Tsc2 knockout (Tsc2^{-/-}) MEFs (FIG. 12G). Nevertheless, livers from Raptor^{fl/fl} mice injected with AAV-TBG-Cre, to induce hepatocyte-specific Raptor deletion, showed reduced PHLPP2 levels (FIG. 13C). Thus, Raptor, but not mTORC1/2 activity, is required for PHLPP2 regulation.

Interestingly, Raptor overexpression did not increase Phlpp2 mRNA in either liver or primary hepatocytes (FIG. 13D and FIG. 12H), even as it induced protein levels of both basal and exogenous PHLPP2 (FIG. 13E). Given the discordant PHLPP2 protein and mRNA expression patterns, it was hypothesized that Raptor may affect PHLPP2 stability, which was tested by treatment with cycloheximide (CHX), an inhibitor of protein translation. As predicted, Raptor overexpression maintained PHLPP2 levels early in the face of CHX treatment (FIG. 13F). Although this effect wanes with prolonged CHX exposure, this data suggests that free Raptor protects PHLPP2 from degradation. Correspondingly, treatment with the proteosomal inhibitor MG-132 revealed that PHLPP2 ubiquitination is blocked by Raptor overexpression (FIG. 13G). Next, the mechanism for this proteosomal-mediated PHLPP2 degradation was investigated, with the hypothesis that it may be a substrate of β-TrCP (beta-transducin repeat-containing protein). β-TrCP is an E3 ligase that serves as the substrate recognition subunit in the SCF (Skp1-Cullin 1-F-box protein) protein complex shown to mediate PHLPP1 degradation in tumors, via the PP2C domain conserved in both PHLPPs (Li et al., 2009). As hypothesized, Raptor overexpression markedly diminishes MG-132-exposed PHLPP2-β-TrCP association (FIG. 13H). Importantly, this results in increased interaction of PHLPP2 with Akt in adult mice (FIG. 13I). The absence of effect of free Raptor on PHLPP1 levels and Akt interaction proves the specificity of this interaction, and closes the molecular circuit between Raptor-β-TrCP and downstream effects on Akt activity by PHLPP2.

### EXAMPLE 9

Hepatic PHLPP2 Levels Decline in Parallel with Free Raptor with Aging and in Obesity

These data prove the necessity of Raptor to maintain normal PHLPP2 levels. Thus, it was hypothesized that the decline in free Raptor levels with aging and obesity would post-transcriptionally reduce PHLPP2 levels, and explain excessive DNL in these states. Indeed, despite unchanged Phlpp2 gene expression (FIG. 15A), liver PHLPP2 protein levels decline with aging (FIG. 15B). PHLPP2 protein levels are similarly lower in dietary (FIG. 15C) or genetic (FIG. 15D) models of obesity, suggesting that decreased free Raptor-driven PHLPP2 stability may explain increased Akt activity and hepatic TG in aging or nutrient excess.

### EXAMPLE 10

PHLPP2 Overexpression Prevents Aging/Obesity-Induced Hepatic Steatosis

Next, it was tested whether exogenous rescue of PHLPP2 expression would inhibit hepatic lipid accumulation by aging and obesity in vivo. Adult HFD-fed mice were transduced with adenovirus expressing control (Ad-GFP) or PHLPP2 (Ad-PHLPP2) to induce moderate liver-specific PHLPP2 overexpression (FIG. 15E). Ad-PHLPP2 mice showed normal body weight, adiposity, hepatic protein and cholesterol content (FIGS. 14A-14D), but as expected, decreased Akt Ser473 phosphorylation (FIG. 15E). Similar to Ad-Raptor mice, Ad-PHLPP2 mice showed decreased liver weight (FIG. 15F), as well as a trend towards lower hepatic TG (FIG. 15G) and lipogenic gene expression (FIG. 15H), and an accompanying decrease in plasma TG (FIG. 15I) as compared to control mice. Further, and again consistent with Ad-Raptor mice, Ad-PHLPP2 mice showed normal glucose tolerance (FIG. 15J) and similar glucose and insulin levels as compared to control mice (FIGS. 14E and 14F). Collectively, these results prove that hepatic PHLPP2 negatively regulates Akt lipogenic activity, while leaving intact Akt repression of hepatic glucose production.

### Discussion—Examples 1-10

Canonical mechanistic target of rapamycin complex 1 (mTORC1) signaling, defined by Raptor-mediated scaffolding of mTOR kinase with substrate, is necessary for obesity-induced hepatic steatosis. Levels of mTORC1-independent (“free”) Raptor were found to decline with aging and obesity, and that exogenous rescue of free Raptor reduces hepatic triglyceride content by interfering with β-TrCP-mediated degradation of the Akt phosphatase, PHLPP2. Whereas PHLPP2 levels are reduced by both aging and nutrient excess, modest Raptor overexpression protects normal PHLPP2-Akt interaction to promote Akt Ser473 dephosphorylation, reducing hepatic de novo lipogenesis without adversely affecting glucose tolerance. Commensurately, forced PHLPP2 expression reverses hepatic steatosis in diet-induced obese mice. The data herein suggest that timely termination of Akt signaling is necessary to maintain lipid homeostasis in aging and obesity, and uncovers the first-described role of mTORC1-independent Raptor (or “free Raptor”) as a regulator of PHLPP2, a novel therapeutic target for non-alcoholic fatty liver disease (NAFLD).

The mechanistic target of rapamycin (mTOR) is a phosphatidylinositol 3-kinase (PI3K)-like serine/threonine kinase that has an essential role in cell growth in all eukaryotes, as well as a recently discovered regulatory role in lipid homeostasis. Mice with liver-specific deletion of the mTOR complex 1 (mTORC1)-defining subunit Raptor (L-Raptor) show reduced SREBP (sterol regulatory element-binding protein)-1c-dependent de novo lipogenesis (DNL) and are thus protected from fatty liver (Peterson et al., 2011). Inhibition of mTOR/Raptor interaction with the allosteric mTORC1 inhibitor rapamycin similarly blocks insulin-dependent SREBP1c activation in primary rat hepatocytes and liver (Brown et al., 2007; Porstmann et al., 2008; Yecies et al., 2011). Consistently, sterol regulatory elements (SREs) are highly enriched in promoters of rapamycin-sensitive genes (Duvel et al., 2010), leading to the conclusion that mTORC1 is required for the lipogenic actions of insulin.

Surprisingly, mice with liver-specific deletion of Tsc1 (L-Tsc1), the endogenous inhibitor of mTORC1, are also spared from both age- and diet-induced hepatic steatosis (Yecies et al., 2011). The molecular mechanism underlying this apparent contradictory result was discovered to be feedback inhibition preventing normal activation of the serine-threonine kinase Akt (Yecies et al., 2011), which then interferes with SREBP1c function via modulation of Insig2a expression (Yabe et al., 2002; Yabe et al., 2003). Akt is a critical molecular node in insulin signal transduction to both gluconeogenic and lipogenic liver pathways (Li et al., 2010; Taniguchi et al., 2006; Yecies et al., 2011). As such, mice lacking both hepatic Akt isoforms (L-Akt1:Akt2) show marked defects in both insulin-mediated metabolic processes in liver-glucose intolerance (Lu et al., 2012), primarily but not exclusively due to loss of its inhibitory action on the gluconeogenic transcription factor FoxO1 (Lin and Accili, 2011), as well as a similar reduction in feeding-induced Srebp1c expression as mice lacking hepatic insulin receptor (Haas et al., 2012). Collectively, these results point to Akt activity as a key regulator of insulin action in liver, and suggest that it affects DNL via both mTORC1-dependent and -independent mechanisms.

Akt is primarily regulated by post-transcriptional means—after constitutive co-translational phosphorylation at Thr450 by mTORC2 (mTOR complex 2) (Ikenoue et al., 2008; Oh et al., 2010), growth factor- or insulin-stimulation of PI3K induces the production of phosphatidylinositol-3,4,5-trisphosphate (PI3P) which recruits Akt to the plasma membrane. Membrane localization unmasks Thr308 in the Akt activation loop for phosphorylation by PDK1, which triggers an additional mTORC2-mediated (Sarbassov et al., 2005) phosphorylation at Ser473 (Leslie et al., 2001; Stephens et al., 1998). The necessity for both T308/S473 stimulatory phosphorylation events in full activation of Akt is illustrated by the phenotype of mice with a liver-specific deletion of the mTORC2-defining subunit Rictor (L-Rictor). L-Rictor mice show absent Akt S473 phosphorylation, leading to FoxO1 hypophosphorylation and resultant excessive hepatic glucose production, as well as reduced hepatic TG, due to a complete loss of refeeding-induced Srebp1c activity and DNL (Hagiwara et al., 2012; Yuan et al., 2012). As expected, both glucose and lipid phenotypes were rescued by restoration of hepatic Akt activity (Hagiwara et al., 2012).

Even as the knowledge of upstream activators of Akt has advanced, the mechanisms accounting for termination or negative regulation of Akt signaling are less well-established. Altered mTORC1 complex stability has been shown to be associated with increased Akt activity (Pajvani et al., 2013)—although this phenotype may be attributed to circumventing normal nutrient-regulated mechanisms of mTORC1 catalytic activity and feedback regulation of Akt (Kim et al., 2002), we hypothesized that mTORC1 stability has an additional, indirect role to terminate the insulin/Akt signal. Herein, it is shown that Raptor exists in both the mTORC1-bound and -independent (“free”) state in livers from young, healthy mice, but that free Raptor levels decline markedly in aging and obesity, coupled with increased Akt activity and hepatic TG. Moderate hepatic Raptor overexpression to correct the free Raptor deficit reversed Akt hyperactivity, decreasing DNL and hepatic TG accumulation by increasing protein stability and Akt association of the recently identified Akt Ser473 phosphatase Pleckstrin homology (PH) domain leucine-rich repeat protein phosphatase 2 (PHLPP2) (Brognard et al., 2007; Gao et al., 2005). Raptor overexpression prevents aging- or high-fat diet (HFD)-mediated reductions in PHLPP2, without adversely affecting either mTORC1/2 activity or whole-body glucose homeostasis. Consistently, mild hepatic PHLPP2 overexpression reduced lipogenic gene expression and reversed obesity-induced fatty liver, without affecting glucose tolerance. These studies identify an unexpected intersection in the intertwined Akt/mTORC1 pathways to regulate hepatic TG content. Furthermore, the apparent selectivity of the PHLPP2 termination of Akt signal to prevent excessive fatty acid synthesis without affecting gluconeogenesis suggests an important modulatory action in the bifurcation model of hepatic insulin action.

Although mTOR and Raptor expression tends to be tightly linked (Kim et al., 2002), at fairly static levels in aging and obesity (Kim and Pajvani, unpublished observations), the association between these catalytic and primary scaffolding subunits of the mTORC1 complex is far more dynamic. Elegant biochemistry has shown that amino acid deprivation tightens, while rapamycin treatment loosens but does not break the mTOR-Raptor interaction (Kim et al., 2002; Kim et al., 2003). In this study, similar biochemical assays were used to prove the existence of mTORC1-independent Raptor, consistent with in vitro observations from other labs (Kaizuka et al., 2010; Menon et al., 2014), as well as modulation of free Raptor levels by pathophysiologic (aging, obesity) stimuli. What upstream signals (i.e., alternative protein-protein interaction, post-translational modification on Raptor or another mTORC1 complex member, etc.) govern these event(s), and whether free Raptor affects other cellular processes also remains to be seen. Nevertheless, this first report of mTORC1-independent activity suggests that the role of Raptor as a simple scaffold for mTOR catalytic activity is an oversimplification, and should prompt re-examination of the phenotypes of tissue-specific Raptor knockout mice.

A conceptually attractive hypothesis of how free Raptor protects PHLPP2 from proteosomal degradation is that insulin-Akt signaling, via free Raptor, may be responsible for its own regulation by PHLPP2, either directly or indirectly. In fact, several predicted Akt and GSK3β (not shown) phosphorylation sites exist in the shared PP2C domain of PHLPP1 and PHLPP2, which compose a β-TrCP-mediated destruction motif (phosphodegron) (Frescas and Pagano, 2008). Thus, an Akt-dependent feedback loop may indeed exist to regulate this interaction and determine the observed posttranslational PHLPP2 regulation in aging or obesity.

The Raptor/PHLPP2 axis provides yet another mechanism of interplay, albeit indirect, between the insulin/Akt and nutrient/mTOR pathways (Hagiwara et al., 2012; Sarbassov et al., 2005; Yecies et al., 2011). Insulin and nutrient-regulated Akt activation by PDK1 and mTORC2 (Alessi et al., 2009; Sarbassov et al., 2005) is subject to negative feedback by TSC1/2-mediated increase in mTORC1 activity via inhibitory phosphorylation of IRS1/2 (Harrington et al., 2004; Shah et al., 2004), but our data suggests an additional layer of regulation by PHLPPs. PHLPPs have been shown to slow Akt-dependent cancer progression (Ghalali et al., 2014; Li et al., 2014; Newton and Trotman, 2014; Wang et al., 2014), but the present study is the first to demonstrate their potential impact in obesity-related metabolic disease and isoform specificity of expression and action in livers. Knockdown of PHLPP2, but not PHLPP1, improves glucose tolerance but simultaneously induces fatty liver, phenocopying mice transduced with myrAkt (Calvisi et al., 2011; Hagiwara et al., 2012; Li et al., 2013), suggesting that endogenous PHLPP2 activity is required to terminate Akt signaling in livers. Conversely, a modest increase in PHLPP2 levels induced by Raptor-induced stabilization, or by adenoviral PHLPP2 transduction, reduces Akt activity and DNL, presumably through mTORC1-independent regulation of Insig2a (Yecies et al., 2011), but remarkably, does not impair glucose homeostasis. Integrating data from liver-specific Rictor knockout mice (Hagiwara et al., 2012) which are unable to maximally activate Akt and show consequent glucose intolerance, we propose a new model of insulin action (FIG. 16) whereby Akt must be activated to avoid aging/obesity-induced T2D, but also inactivated by PHLPP2 in a timely fashion to avoid NAFLD. Our model is consistent with the “bifurcation” model of insulin signaling proposed by Brown & Goldstein (Li et al., 2010), and others (Haas et al., 2012), but differs in the kinetics of events—inhibition of FoxO1 is an “early” event and requires the full force of Akt action, but an extension of Akt activity by maintained S473 phosphorylation induces the “late” lipogenic response by mTORC1-dependent (Li et al., 2010) and -independent (Yecies et al., 2011) pathways. PHLPPs have been the subject of considerable research in recent years, due to their therapeutic potential in neoplastic disease. Loss-of-heterozygosity has been observed at both PHLPP loci in multiple solid tumors (Newton and Trotman, 2014) and a common PHLPP2 loss-of-function variant shown to reduce Akt dephosphorylation (Brognard et al., 2009) has been observed in high-grade breast cancers. In combination with the known oncogenic role of Akt, these findings suggest that PHLPPs may have tumor suppressor activity. Enthusiasm for pharmacologic PHLPP activators, such as HDAC3 inhibitors (Bradley et al., 2013) or adenylate cyclase activators (Gao et al., 2009), has been tempered by speculated interference with normal Akt function and potential to cause new-onset T2D (Newton and Trotman, 2014). The data would suggest these fears may be unfounded, that increase in hepatic PHLPP2 levels/activity dissociate Akt functions in liver, and thus, extend the therapeutic potential of PHLPP2 activators to stem the tide of obesity-induced NAFLD.

### EXAMPLE 11

mTORC1-Independent Raptor Prevents Hepatic Steatosis by Stabilizing PHLPP2

Mechanistic target of rapamycin complex 1 (mTORC1), defined by the presence of Raptor, is an evolutionarily conserved and nutrient-sensitive regulator of cellular growth and other metabolic processes. While all known functions of Raptor involve its scaffolding mTOR kinase with substrate, herein we report the first-described role of mTORC1-independent (“free”) Raptor to negatively regulate hepatic Akt activity and lipid metabolism. Free Raptor levels in liver decline with aging and obesity; correction of this free Raptor deficit reduces liver triglyceride content, through reduced β-TrCP-mediated degradation of the Akt phosphatase, PHLPP2. Commensurately, forced PHLPP2 expression ameliorates hepatic steatosis in diet-induced obese mice. These data suggest that the balance of free and mTORC1-associated Raptor govern hepatic lipid accumulation, and uncover the potentially therapeutic role of PHLPP2 activators in non-alcoholic fatty liver disease (NAFLD).

Obesity-induced metabolic dysfunction manifests as multiple chronic medical conditions, including Type 2 Diabetes (T2D) and Nonalcoholic fatty liver disease (NAFLD) (Ford et al., 2002). NAFLD contributes to the overall cardiovascular risk of obesity (Villanova et al., 2005), but is also the most common chronic liver disease, predisposing to cirrhosis and hepatocellular carcinoma (Dowman et al., 2011). Aging and obesity are well-established risk factors for NAFLD (Slawik and Vidal-Puig, 2006), but the molecular mechanism underlying this risk is poorly defined, precluding specific pharmacologic strategies to target excess hepatic triglycerides (TG).

The evolutionarily conserved mechanistic target of rapamycin (mTOR) is a phosphatidylinositol 3-kinase (PI3K)-like serine/threonine kinase that regulates cell growth in response to nutrient signals, as well as a recently discovered regulatory role in lipid homeostasis. Mice with liver-specific deletion of the mTOR complex 1 (mTORC1)-defining subunit Raptor (L-Raptor) show reduced SREBP (sterol regulatory element-binding protein)-1c-dependent de novo lipogenesis (DNL) and are thus protected from fatty liver (Peterson et al., 2011). Inhibition of mTOR/Raptor interaction with the allosteric mTORC1 inhibitor rapamycin similarly blocks insulin-dependent SREBP1c activation in primary rat hepatocytes and liver (Porstmann et al., 2008, Brown et al., 2007, Yecies et al., 2011). Consistently, sterol regulatory elements (SREs) are highly enriched in promoters of rapamycin-sensitive genes (Duvel et al., 2010), leading to the conclusion that mTORC1 is required for the lipogenic actions of insulin.

Surprisingly, mice with liver-specific deletion of Tsc1 (L-Tsc1), the endogenous inhibitor of mTORC1, are also spared from both age- and diet-induced hepatic steatosis (Yecies et al., 2011). The molecular mechanism underlying this apparent contradictory result was discovered to be feedback inhibition preventing normal activation of the serine-threonine kinase Akt (Yecies et al., 2011), which then interferes with SREBP1c function via modulation of Insig2a expression (Yabe et al., 2002, Yabe et al., 2003). Akt is a critical molecular node in insulin signal transduction to both gluconeogenic and lipogenic liver pathways (Yecies et al., 2011, Yabe et al., 2003, Li et al., 2010). As such, mice lacking both hepatic Akt isoforms show marked defects in both insulin-mediated metabolic processes in liver-glucose intolerance (Lu et al., 2012), primarily but not exclusively due to loss of its inhibitory action on the gluconeogenic transcription factor FoxO1 (Lin and Accili, 2011), as well as a similar reduction in feeding-induced Srebp1c expression as mice lacking hepatic insulin receptor. Collectively, these results point to Akt activity as a key regulator of insulin action in liver, and suggest that it affects DNL via both mTORC1-dependent and -independent mechanisms (Haas et al., 2012).

Akt is primarily regulated by post-transcriptional means—insulin-stimulation of PI3K induces membrane localization, unmasking Thr308 for phosphorylation by PDK1 which then triggers mTORC2 (mTOR complex 2)-mediated phosphorylation at Ser473 (Stephens et al., 1998, Leslie et al., 2001). While the necessity for both T308/S473 phosphorylation events in physiologic Akt activation is well-established (Yuan et al., 2012, Hagiwara et al., 2012, Mora et al., 2005), mechanisms accounting for termination or negative regulation of Akt signaling are far less understood. Our lab has shown that altered mTORC1 complex stability is associated with increased Akt activity (Mora et al., 2005)—although this phenotype may be attributed to circumvention of normal nutrient-regulated mechanisms of mTORC1 catalytic activity and feedback regulation of Akt (Kim et al., 2002), we hypothesized that mTORC1 stability has an additional, indirect role to terminate the Akt signal. Herein, we show that Raptor exists in both the mTORC1-bound and -independent (“free”) state in liver of young, healthy mice, but that free Raptor levels decline markedly in aging and obesity, coupled with increased Akt activity and hepatic TG. Moderate hepatic Raptor overexpression to correct the free Raptor deficit reversed Akt hyperactivity, decreasing DNL and hepatic TG accumulation by increasing protein stability of the recently identified Akt Ser473 phosphatase Pleckstrin homology (PH) domain leucine-rich repeat protein phosphatase 2 (PHLPP2) (Gao et al., 2005, Brognard et al., 2007). Raptor overexpression prevents aging- or high-fat diet (HFD)-mediated reductions in PHLPP2 levels, without adversely affecting either mTORC1/2 activity or whole-body glucose homeostasis. Consistently, mild hepatic PHLPP2 overexpression reduced lipogenic gene expression and obesity-induced TG accumulation, without affecting glucose tolerance. These data suggests an important modulatory action of the balance in free and mTORC1-bound Raptor in the bifurcation model of hepatic insulin action, as well as point to PHLPP2 as a novel therapeutic target for NAFLD.

“Free” Raptor Levels Decline in Aged and Obese Liver

mTORC1 complexes are affected by hormonal signals and oxidative stress (Kim et al., 2002, Menon et al., 2014), likely through direct effects on mTORC1 subunits, and modulated by other upstream signals, such as IPMK and Notch (Mora et al., 2005, Kim et al., 2011). These data suggest that the mTOR/Raptor interaction is fluid. As liver-specific Raptor (L-Raptor) knockout mice show aging-dependent defects in ketogenesis (Sengupta et al., 2010) and obesity-dependent reductions in lipogenesis (Peterson et al., 2011), we hypothesized that mTORC1 component interaction may be altered in these pathophysiologic processes. We performed size-exclusion chromatography to fractionate detergent-free liver lysate from young mice, and in parallel, older or leptin-deficient (ob/ob) obese mice. In all three groups of mice, mTOR and GPI, eluted in high-molecular weight (HMW, ^{˜}800 kDa) fractions (FIGS. 17a,b), consistent with mTORC1 complex dimers (Menon et al., 2014, Yip et al., 2010, Jain et al., 2014). Raptor was also found mostly in HMW fractions in older or obese mice, but livers from young mice showed a surprising amount of “free” (^{˜}150 kDa) Raptor (FIGS. 17a,b). We next sought confirmation of this finding using alternative biochemical methods. We exposed liver lysate to a cell-permeable, non-cleavable chemical cross-linker to trap native mTORC1 complexes, and again observed a marked decrease of free Raptor with aging or obesity, without change in other mTORC1/2 component distribution (FIGS. 17c,d and FIGS. 23a-c). As total levels of mTOR and Raptor did not substantially change, we hypothesized that altered free Raptor reflected greater mTOR-Raptor association in aging or obesity. To test this, we exposed liver lysate to a cleavable cross-linker to ensure complete recovery of mTORC1 components. As predicted, whereas the interaction between mTOR and GPL was unchanged, mTOR-Raptor association was markedly increased in ob/ob mice (FIG. 23d). Consistently, performing anti-mTOR immunoprecipitation (IP) using CHAPS detergent to sustain the mTOR-Raptor interaction in the absence of cross-linker (Kim et al., 2002), we found greater bound Raptor in ob/ob mice (FIG. 23e). Although the disappearance of free Raptor in liver from aging or obese mice was consistent across multiple experimental platforms, we remained skeptical, as various in vitro manipulations have been shown to affect biochemical recovery of mTORC1 complexes without true impact on mTOR-Raptor binding (Kim et al, 2002, Kim et al., 2003). For instance, transient amino acid deprivation increases, while rapamycin treatment decreases IP efficiency of Raptor (FIG. 23f), but neither stimulus altered free Raptor levels (FIG. 17e). This suggests that our observation of free Raptor in young liver is likely through a novel mechanism, and that nutrient and/or hormonal signals prompts a true change in Raptor-mTOR association. We investigated this possibility in vitro, to dissociate the various components of obesity and found that while transient or chronic hyperglycemia and brief insulin exposure were ineffectual, prolonged high-dose insulin treatment to induce insulin resistance (Cook et al., 2015) reduced free Raptor (FIG. 17f and FIG. 23e). Thus, hepatocyte insulin resistance is sufficient to reduce free Raptor levels.

Rescue of Free Raptor Levels Prevents Aging and Obesity-Dependent Hepatic Steatosis

We next hypothesized that loss of free Raptor in the insulin-resistant state, beyond the above correlation, may be causative to aging/obesity-induced metabolic dysfunction, which in liver manifests as excessive triglyceride (TG) content, or hepatic steatosis. To test this, we transduced adult mice with adenovirus encoding Raptor (Ad-Raptor) or GFP control (Ad-GFP) to acutely increase the hepatic Raptor/mTOR ratio, and thus free Raptor levels (FIG. 18a). As a further internal control, we transduced young mice, which have “normal” free Raptor levels (FIG. 24a). While we observed no benefit in young mice, suggestive of a threshold effect of free Raptor, aging-induced hepatic TG accumulation was blocked by Ad-Raptor delivery (FIG. 18b). To push the hypothesis further, we repeated the experiment in aged (12-month-old) or diet-induced obese (DIO) mice, which have further elevations in hepatic TG as compared to young or adult mice (FIG. 24b). Remarkably, acute increase in free Raptor levels reduced liver TG and liver weight in both aged (FIG. 2c and FIG. 24c) and DIO mice (FIG. 18d and FIG. 24d), proving that the observed free Raptor deficit in these states has biological consequence, as replacement can ameliorate aging/obesity-induced hepatic steatosis.

Given the known hepatocyte tropism of adenovirus (Kim et al., 2002), we hypothesized that free Raptor protects from steatosis through a cell-autonomous mechanism, consistent with unchanged body weight, adiposity and plasma fatty acids in Ad-Raptor mice (FIGS. 25a-c). Liver Acox and Cpt1a gene expression and plasma ketone levels were unchanged (FIGS. 25d-e), indicative of normal β-oxidation/ketogenesis. Plasma TG was lower in Ad-Raptor mice (FIG. 18e), implying no increase in hepatic TG secretion as VLDL. Finally, to test de novo lipogenesis, we injected ³H2O into Ad-Raptor and control mice, and measured incorporation of label into newly synthesized hepatic fatty acid. We found that adult mice had higher rates of lipogenesis than young mice, but this was reversed by correcting the aging-related free Raptor defect (FIG. 18f). Consistently, we observed reduced expression of Srebp1c (sterol regulatory element-binding protein-1c), the master regulator of lipogenesis, as well as its transcriptional targets in Ad-Raptor-transduced liver (FIG. 2g) and primary hepatocytes (FIG. 25f).

Increase in Free Raptor Levels Reduces Akt-Mediated Lipogenesis Independent of mTORC1/2

We next investigated the molecular mechanism underlying free Raptor-mediated reduced lipogenesis. Data from rapamycin-treated hepatocytes and from L-Raptor mice has proven that mTORC1 activity is necessary for normal hepatic Srebp1c function (Peterson et al., 2011, Brown et al., 2007). If Raptor overexpression reduces mTORC1 catalytic function in liver, similar to in vitro effects observed in HEK293 cells (Kim et al., 2002), we would expect impaired Srebp1c-dependent lipogenesis. Ad-Raptor-transduced liver, however, showed normal fasted or refed mTORC1 kinase activity on recombinant 4E-BP1, phosphorylation of canonical mTORC1 targets and hepatic protein content (FIG. 26a-c, FIG. 60), in stark contrast with the phenotype of L-Raptor mice (Sengupta et al., 2010). Consistently, mTORC1-dependent negative feedback on insulin signaling through IRS1 phosphorylation was unchanged (FIG. 26d), suggesting that known effectors of hepatic mTORC1 activation likely trump the effects of Raptor overexpression. We next tested an alternative hypothesis whereby the addition of excess Raptor could “steal” shared mTOR components from mTORC2, and recapitulate the lower hepatic TG observed in L-Rictor mice21, but we observed normal mTOR interaction with Rictor and the shared mTORC1/2 component, GβL (FIG. 26e). In addition, Ad-Raptor mice showed normal p-Akt Thr450 and PKCα levels (FIGS. 26f, g), which are phosphorylated and stabilized by mTORC2 (Facchinetti et al., 2008, Ikenoue et al., 2008).

Interestingly, we did find that phosphorylation of Akt at Ser473, another known mTORC2 substrate, was increased by aging in Ad-GFP, but not Ad-Raptor mice (FIG. 19a). PI3K/PDK1-mediated phosphorylation at Thr308 was unaffected (FIG. 19b), but we predicted that decreased Akt S473 phosphorylation in Ad-Raptor mice would be sufficient to reduce Akt kinase activity (Sarbassov et al., 2005). Indeed, GSK3β Ser9 (FIG. 27a) and general Akt substrate phosphorylation (FIG. 27b) was lower, as was immunoprecipitated Akt kinase activity on recombinant GSK3β (FIG. 19c). Additionally, reduced Akt activity in Ad-Raptor livers led to a 2-fold increase in hepatic Insig2a expression (FIG. 27c), supporting the conclusion that free Raptor regulates Srebp1c-mediated lipogenesis via an Akt-regulated but mTORC1-independent pathway (Yecies et al., 2011).

Raptor Increases PHLPP2 Levels to Terminate Akt Signaling and Reduce Liver TG

Akt Ser473 phosphorylation was not altered in extra-hepatic tissues of Ad-Raptor mice (FIG. 27d), suggesting a cell-autonomous effect of free Raptor on Akt activity, as opposed to a whole-body change in insulin sensitivity, consistent with normal plasma insulin levels (FIG. 27e). These data prompted the hypothesis that Akt S473 dephosphorylation was increased in Ad-Raptor mice. PHLPPs (PH domain leucine-rich repeat protein phosphatases), encoded by Phipp1, with two splice variants (PHLPP1α and PHLPP1β) and Phlpp2, have recently been shown to dephosphorylate Akt Ser473 to terminate insulin/growth factor action (Gao et al., 2005, Brognard et al., 2007). PHLPP1β and PHLPP2 are expressed in liver (FIG. 28a), but Raptor overexpression selectively increased PHLPP2 (FIG. 19d). PHLPP2 levels were unaffected by mTOR inhibitors (FIG. 19e and FIG. 28b) or increased mTORC1 activity in Tsc2 knockout (Tsc2−/−) MEFs (FIG. 28c), but are significantly reduced in livers of adult or HFD-fed L-Raptor mice (FIGS. 28d,e) and in shRaptor-transduced primary hepatocytes (FIG. 28f). Conversely, acute mTOR knockdown to increase the Raptor/mTOR ratio and thus free Raptor levels, increased hepatocyte PHLPP2 (FIG. 19f). In sum, these data prove that free Raptor, but not mTORC1/2 activity, regulates PHLPP2.

Interestingly, Raptor overexpression did not increase Phlpp2 mRNA in liver or primary hepatocytes (FIGS. 28g,h), even as it induced protein levels of both basal and exogenous PHLPP2 (FIG. 28i), suggesting an impact of free Raptor on PHLPP2 stability. Consistent with this hypothesis, Raptor overexpression maintained PHLPP2 levels in the face of cycloheximide treatment (FIG. 19g), likely due to reduced ubiquitin-mediated proteosomal degradation (FIG. 3h). β-TrCP (beta-transducin repeat-containing protein), the E3 ligase/substrate recognition subunit of the SCF (Skp1-Cullin 1-F-box protein) protein complex, has been shown to mediate PHLPP1 degradation in tumors—as this regulation occurs via the PP2C domain conserved in both PHLPPs (Li et al., 2009), we tested PHLPP2β-TrCP association in primary hepatocytes and hepatoma cells. Raptor overexpression diminished proteosomal inhibitor-exposed PHLPP2-β-TrCP association (FIG. 19i and FIG. 28j), while PHLPP1 levels (FIG. 19d) and its association with β-TrCP (not shown) was not altered, suggesting the specificity of free Raptor's protection of PHLPP2 from β-TrCP-mediated proteosomal degradation.

Hepatic PHLPP2 Levels Decline in Parallel with Free Raptor with Aging and in Obesity

Free Raptor levels decline with aging and obesity—consistently, despite unchanged hepatic Phlpp2 gene expression (FIG. 20a), we observed lower PHLPP2 protein levels with aging and in dietary or genetic models of obesity (FIGS. 20b-d and FIGS. 33a-d). To test whether reduction in hepatic PHLPP2 is sufficient to induce steatosis, we constructed adenoviruses encoding shRNA targeting either Phlpp1 (Ad-shPHLPP1) or Phlpp2 (Ad-shPHLPP2). In primary hepatocytes, knockdown of PHLPP2 but not PHLPP1 increased Akt Ser473 phosphorylation (FIG. 29a). Correspondingly, mice transduced with Ad-shPHLPP2 showed increased liver weight and hepatic steatosis (FIGS. 29b,c), mirroring the phenotype of mice transduced with constitutively active Akt (Ad-myrAkt) (Calvisi et al., 2011, Li et al., 2013). The converse experiment was performed to test whether adenoviral rescue of PHLPP2 (Ad-PHLPP2) in DIO mice would inhibit hepatic lipid accumulation. As expected, Ad-PHLPP2 mice showed decreased Akt Ser473 phosphorylation (FIG. 21e). In addition, despite unchanged body weight and adiposity (FIGS. 30a,b), Ad-PHLPP2 mice showed decreased liver weight (FIG. 30c) and a trend towards lower hepatic TG (FIG. 21f) and lipogenic gene expression (FIG. 21g), with an accompanying decrease in plasma TG (FIG. 21h).

These data suggest that preservation of free Raptor, via PHLPP2, can prevent hepatic steatosis, but to prove this, we co-transduced Ad-Raptor (or Ad-GFP control) mice with Ad-shControl, Ad-shPHLPP1 or Ad-shPHLPP2 adenoviruses. PHLPP2 knockdown restored Akt Ser473 phosphorylation in Ad-Raptor mice (FIG. 22a), resulting in hepatic lipogenic gene expression, as well as hepatic and plasma TG comparable to control mice (FIGS. 22b-d), without altering body weight/adiposity, serum metabolites (FIGS. 31a-d) or canonical mTORC1/2 signaling or activity (FIG. 22a and FIG. 31e). Interestingly, Ad-myrAkt transduction in Ad-Raptor mice similarly increased liver TG (FIG. 22e), buttressing our conclusion that free Raptor maintains normal hepatic lipogenesis/TG content by increasing PHLPP2, to restrict Akt activation. Reduced hepatic Akt activity is typically associated with hyperglycemia (Ono et al., 2003). Surprisingly, but consistent with the phenotype of Ad-Raptor mice (FIGS. 32a-c), Ad-PHLPP2 mice showed normal gluconeogenic gene expression (not shown) and glucose tolerance (FIG. 5f), as well as similar glucose and insulin levels as compared to control mice (FIGS. 32d,e). These data suggest that free Raptor, via PHLPP2, selectively regulates Akt's effects on lipogenesis, while leaving intact Akt repression of hepatic glucose production.

### Discussion—Example 11

Although mTOR and Raptor expression tends to be tightly linked (Kim et al., 2002), the association between these catalytic and primary scaffolding subunits of the mTORC1 complex is far more dynamic. Elegant biochemistry has shown that amino acid deprivation tightens, and rapamycin treatment loosens but does not break the mTOR-Raptor interaction (Kim et al., 2002, Kim et al., 2003). While in vitro studies have hinted of the presence of an mTORC1-independent Raptor species (Menon et al., 2014, Kaizuka et al., 2010), the data herein proves for the first time the existence, and modulation by pathophysiologic (aging, obesity) stimuli, of free Raptor. Further study is required to determine whether free Raptor exists due to failure in association or true dissociation from mTOR, and whether the free and mTORC1-bound Raptor pools may interchange. In addition, what upstream signals (i.e., alternative protein-protein interaction, post-translational modification on Raptor or another mTORC1 complex member, etc.) govern these event(s), and whether free Raptor affects other cellular processes are important questions that our lab is actively pursuing. Nevertheless, this first report of mTORC1-independent Raptor function suggests that its heretofore accepted role as a simple scaffold for mTOR catalytic activity is an oversimplification, and should prompt re-examination of the phenotypes of tissue-specific Raptor knockout mice.

The Raptor/PHLPP2 axis provides yet another mechanism of interplay, albeit indirect, between the insulin/Akt and nutrient/mTOR pathways (Yecies et al., 2011, Hagiwara et al., 2012, Sarbassov et al., 2005). While there are likely additional determinants of hepatic PHLPP2, as evidenced by residual levels in L-Raptor livers, we postulate that insulin-Akt signaling, via free Raptor, may be responsible for its own regulation by PHLPP2. In fact, several predicted Akt and GSK3β phosphorylation sites exist in the C-terminus of PHLPP2 within a β-TrCP-mediated destruction motif (phosphodegron), suggesting an Akt-dependent negative feedback loop to determine posttranslational PHLPP2 stability to regulate Akt activity in obese liver. Conversely, a modest free Raptor-induced increase in PHLPP2 levels can reduce Akt-regulated DNL via increased Insig2a expression, but remarkably, does not impair glucose homeostasis. This is likely due to a specific reduction in Akt activity in the late post-prandial state, in striking contrast to the phenotype of hepatic mTORC2-deficient (and thus completely Akt S473-deficient) L-Rictor mice (Hagiwara et al., 2012), which fail to maximally activate Akt, leading to unchecked hepatic glucose output and glucose intolerance, closely approximating “complete” hepatic insulin resistance (Kaizuka et al., 2010, Dong et al., 2008). Integrating these data, we propose a new model of insulin action (FIG. 23) whereby Akt must be appropriately stimulated, but also inactivated by PHLPP2 in a timely fashion, to maintain normal hepatic physiology. Our model is consistent with the “bifurcation” model of insulin signaling proposed by Brown & Goldstein (Li et al., 2010), and others (Haas et al., 2012), but differs in the kinetics of events—inhibition of FoxO1 to repress gluconeogenesis is an “early” event and requires the full force of Akt action45, but an extension of Akt activity by maintained S473 phosphorylation induces the “late” lipogenic response by mTORC1-dependent (Li et al., 2010) and -independent (Yecies et al., 2011) pathways.

PHLPPs have been the subject of considerable research in recent years, due to their therapeutic potential in neoplastic disease. Loss-of-heterozygosity has been observed at both PHLPP loci in multiple solid tumors (Newton and Trotman, 2014) and a common PHLPP2 loss-of-function variant shown to reduce Akt dephosphorylation (Brognard et al., 2009) has been observed in high-grade breast cancers. In combination with the known oncogenic role of Akt, these findings suggest that PHLPPs may have tumor suppressor activity. Enthusiasm for pharmacologic PHLPP activators, such as HDAC3 inhibitors (Bradley et al., 2013) or adenylate cyclase activators49, has been tempered by speculated interference with normal Akt function and potential to cause new-onset T2D (Newton and Trotman, 2014), consistent with epidemiologic studies showing increased PHLPP1, but importantly not PHLPP2, expression in adipose or muscle of obese/T2D (Andreozzi et al., 2011, Cozzone et al., 2008). Conversely, our gain- and loss-of-function studies show that PHLPP1 has little function in liver, whereas hepatic PHLPP2 levels/activity dissociate Akt functions in liver to reduce lipogenesis without adversely affecting glucose tolerance. Our data would suggest that application of putative PHLPP2 agonists or liver-specific delivery of non-specific PHLPP activators (Mishra et al., 2013) have the potential to stem the tide of obesity-induced NAFLD.

### EXAMPLE 12

Identification of Post-Translational Modifications (PTMs) on PHLPP2 Protein

Reduced hepatic PHLPP2 protein levels, but not Phlpp2 gene expression, were observed in aging/obesity and short-term refeeding, suggesting altered PHLPP2 stability in the insulin-resistant or insulin-rich liver, consistent with data showing regulated PHLPP2 ubiquitination and proteosomal degradation. It was next hypothesized that PHLPP2 protein stability was affected by nutrient- or hormone-mediated post-translational modifications (PTMs), and an in vivo unbiased liquid chromatography-tandem mass spectrometry (LC/MS-MS) screen by transducing C57BL/6 wild-type (WT) mice with Ad-HA-Flag-PHLPP2 followed by sequential anti-HA→anti-FLAG IP was performed (FIG. 34). With this approach, five novel phosphorylation sites were identified at the functionally undefined C-terminal region between the PP2C domain and PDZ-binding motif of PHLPP2 (FIG. 35-37).

PHLPP2 Ser1119 and Ser 1210 are Phosphorylated in Response to Glucagon

To identify the signal transduction pathways involved in PHLPP2 phosphorylation, a Phos-tag-based mobility shift assay was used, which magnifies the difference in migration speed of phosphorylated and non-phosphorylated proteins (FIG. 38A-C), and it was found that activation of protein kinase A (PKA) signaling by treatment with either cAMP, or forskolin which activates adenylate cyclase to stimulates cAMP production, increased PHLPP2 phosphorylation (FIG. 39). Next, to validate LC/MS-MS-based phospho-peptide mapping results, each identified Ser/Thr site was individually mutated to Alanine, then transfected WT or Ala-mutant PHLPP2 in hepatocytes, and Ser1119 and Ser1210 phosphorylations was confirmed (FIG. 40A-B). Next, it was found that forskolin could similarly induce phosphorylation overexpressed PHLPP2 in primary hepatocytes (FIG. 41A), essential to test the effects of Ala-mutant PHLPP2 in vitro. Interestingly, WT, S1119A, and S1210A mutant of PHLPP2 were all phosphorylated with forskolin treatment, suggesting that both S1119 and S1210 sites might be phosphorylated by PKA (FIG. 41B). Thus, double mutants were generated, such as S1119/1210 and it was found that ablation of both sites rendered the S1119A/S1210A mutant insensitive to forskolin-induced phosphorylation (FIG. 41C). These data matched in silico analysis of consensus substrate sites for mammalian kinases, as both Ser1119 and Ser1210 are exact matches for the cyclic AMP-PKA consensus site. To investigate the effect of PKA on PHLPP2 phosphorylation in primary hepatocytes, either WT or S1119/1210A mutant of PHLPP2 was immunoprecipitated from cell extracts treated with glucagon or forskolin, and then subjected to immunoblot analysis with an antibody specific for phosphorylated PKA substrate proteins. PKA-mediated phosphorylation of WT, but not S1119A/S1210A mutant PHLPP2 was robustly increased following glucagon or forskolin treatment of primary hepatocytes. In addition, forskolin-mediate PHLPP2 phosphorylation was reduced by pretreatment of PKA inhibitor, H89, proving that PKA stimulates PHLPP2 phosphorylation (FIG. 42A-B).

These data suggest that glucagon may induce PHLPP2 phosphorylation and thus degradation, and that inhibitors of glucagon signaling would increase PHLPP2 levels. To test this concept in vivo, adenoviruses encoding shRNA were transduced to the glucagon receptor (or a control shRNA) to either genetically-induced (db/db) mice or diet-induced obese (DIO) mice. As hypothesized, knockdown of liver glucagon signaling rescues the lower PHLPP2 levels seen in obesity (FIGS. 43A-C and FIGS. 44A-B). This suggests that inhibitors of glucagon signaling will similarly increase the pathologically lower PHLPP2 levels in the obese state, and reduce hepatic and plasma triglyceride.

PHLPP2 Knockout (KO) Cells Show Prolonged Insulin Signaling.

To determine whether PHLPP2 is necessary to terminate insulin signaling, PHLPP2 KO HepG2 hepatoma cells were generated using the CRISPR (Clustered Regularly Interspaced Short Palindromic Repeats)-Cas9 system. Using three different single guide RNA (sgRNA) for PHLPP2, significantly reduced PHLPP2 protein and mRNA levels were observed, without effects on off-target gene expression (FIG. 45A-C). Next, HepG2 stable cell lines were generated expressing the most efficient PHLPP2 sgRNA (#3) sgRNA3 (FIG. 46A) and, in parallel, a mouse hepatoma cell lines, Hepa1c1c7 cells (FIG. 46B). It was found that PHLPP2-deficient cells showed sustained insulin signaling as compared to control cells, as judged by Akt phosphorylation (FIG. 47A). Next, PHLPP2 levels were “rescued” in these CRISPR-induced knockout cell lines with either WT or S1119/1210A PHLPP2, and found that only mutant PHLPP2 prevented acute insulin-induced Akt phosphorylation (FIG. 47B), indicating that PKA-mediated phosphorylation is likely responsible for decreased insulin-mediated Akt phosphorylation.

Identification of Novel PHLPP2 Interaction Proteins

To further identify the regulatory mechanism by which PHLPP2 levels decrease in aging and obese liver through proteosomal degradation, LC/MS-MS analysis was repeated to determine novel PHLPP2 binding partners (FIG. 48), and identified KCTD17 (potassium channel tetramerization domain containing 17) as a strong PHLPP2 interactor. Next, PHLPP2 was confirmed to associate with KCTD17, but in a proteasome-dependent manner (FIG. 49A). Consistent with the notion that S1119/S120 phosphorylation regulates PHLPP2 stability by proteosomal degradation, the association of endogenous PHLPP2 with KCTD17 was significantly enhanced by treatment of forskolin, and even more strongly with simultaneous treatment with forskolin and the proteasome inhibitor, MG132 (FIG. 49B). Finally, and as hypothesized, while WT PHLPP2 associates with KCTD17 with forskolin and MG132 treatment, S1119/1210A mutant PHLPP2 showed markedly lower KCTD17 binding (FIG. 50). In sum, these data indicate that KCTD17 is a novel PHLPP2 interacting protein, which may regulate PHLPP2 degradation in a phosphorylation/proteasome-dependent manner.

### EXAMPLE 13

“Free” Raptor—a Novel Regulator of Metabolism

The established role of Raptor as a scaffold for mTOR catalytic activity is an oversimplification. In fact, mTORC1-independent Raptor (or, Raptor^{free}) stabilizes the Akt phosphatase, PHLPP2 (PH domain leucine-rich repeat-containing proteinphosphatase 2), to prevent fatty liver.

Through complementary biochemical methods, Raptor^{free} was found to be abundant in livers isolated from young, healthy mice, but increased mTOR-Raptor association with aging and obesity, leading to the progressive disappearance of Raptor^{free}. Strikingly, replacement of the Raptor^{free} deficit reduced feeding-associated Akt Ser473 phosphorylation (with no effects on PI3K-induced Akt Thr308 phosphorylation or other upstream stimuli) by preventing proteosomal degradation of PHLPP2, with end result of reduced (de novo lipogenesis) DNL and hepatic triglyceride accumulation. As such, hepatic PHLPP2 levels are also lower in aged or obese mice lacking Raptor^{free}, and PHLPP2 knockdown reversed Raptor^{free}-mediated improvements in DNL/hepatic triglyceride. Consistently, adenoviral PHLPP2 overexpression abrogated obesity-induced fatty liver, surprisingly without adverse effects on glucose homeostasis. These data suggest two novel and complementary aspects of insulin action in liver: 1) the existing bifurcation model of hepatic insulin action6 is missing a critical temporal element—post-prandial Akt activation is necessary to inhibit HGP, but prolonged Akt activity increases DNL if the insulin signal is sustained, without beneficial effects on glucose; and 2) in aging/obesity-induced insulin resistance, loss of hepatocyte Raptor^{free} destabilizes PHLPP2, which allows compensatory hyperinsulinemia to stimulate DNL, that exacerbates NAFLD (FIG. 65).

### REFERENCES

Alessi, D. R., Pearce, L. R., and Garcia-Martinez, J. M. (2009). New insights into mTOR signaling: mTORC2 and beyond. Science signaling 2, pe27.

Andreozzi, F., et al. Increased levels of the Akt-specific phosphatase PH domain leucine-rich repeat protein phosphatase (PHLPP)-1 in obese participants are associated with insulin resistance. Diabetologia 54, 1879-1887 (2011).

Beezhold K, Liu J., Kan, H. Meighan T., Castranova V., Shi X., and Chen F. (2011). miR-190-mediated downregulation of PHLPP contributes to arsenic-induced Akt activation and carcinogenesis. Toxicological sciences 123(2), 411-420.

Bhaskar, P. T., and Hay, N. (2007). The two TORCs and Akt. Developmental cell 12, 487-502.

Bradley, E. W., Carpio, L. R., and Westendorf, J. J. (2013). Histone deacetylase 3 suppression increases PH domain and leucine-rich repeat phosphatase (Phlpp)1 expression in chondrocytes to suppress Akt signaling and matrix secretion. The Journal of biological chemistry 288, 9572-9582.

Brognard, J., Niederst, M., Reyes, G., Warfel, N., and Newton, A. C. (2009). Common polymorphism in the phosphatase PHLPP2 results in reduced regulation of Akt and protein kinase C. The Journal of biological chemistry 284, 15215-15223.

Brognard, J., Sierecki, E., Gao, T., and Newton, A. C. (2007). PHLPP and a second isoform, PHLPP2, differentially attenuate the amplitude of Akt signaling by regulating distinct Akt isoforms. Molecular cell 25, 917-931.

Brown, N. F., Stefanovic-Racic, M., Sipula, I. J., and Perdomo, G. (2007). The mammalian target of rapamycin regulates lipid metabolism in primary cultures of rat hepatocytes. Metabolism: clinical and experimental 56, 1500-1507.

Calvisi, D. F., Wang, C., Ho, C., Ladu, S., Lee, S. A., Mattu, S., Destefanis, G., Delogu, S., Zimmermann, A., Ericsson, J., et al. (2011). Increased lipogenesis, induced by AKT-mTORC1-RPS6 signaling, promotes development of human hepatocellular carcinoma. Gastroenterology 140, 1071-1083.

Cook, J. R., Matsumoto, M., Banks, A. S., Kitamura, T., Tsuchiya, K., and Accili, D. (2015). A Mutant Allele Encoding DNA-Binding-Deficient Foxo1 Differentially Regulates Hepatic Glucose and Lipid Metabolism. Diabetes.

Cozzone, D., et al. Isoform-specific defects of insulin stimulation of Akt/protein kinase B (PKB) in skeletal muscle cells from type 2 diabetic patients. Diabetologia 51, 512-521 (2008).

Dong, X. C., et al. Inactivation of hepatic Foxo1 by insulin signaling is required for adaptive nutrient homeostasis and endocrine growth regulation. Cell metabolism 8, 65-76 (2008)

Dowman, J. K., Armstrong, M. J., Tomlinson, J. W., and Newsome, P. N. (2011). Current therapeutic strategies in non-alcoholic fatty liver disease. Diabetes, obesity & metabolism 13, 692-702.

Duvel, K., Yecies, J. L., Menon, S., Raman, P., Lipovsky, A. I., Souza, A. L., Triantafellow, E., Ma, Q., Gorski, R., Cleaver, S., et al. (2010). Activation of a metabolic gene regulatory network downstream of mTOR complex 1. Molecular cell 39, 171-183.

Facchinetti, V., Ouyang, W., Wei, H., Soto, N., Lazorchak, A., Gould, C., Lowry, C., Newton, A. C., Mao, Y., Miao, R. Q., et al. (2008). The mammalian target of rapamycin complex 2 controls folding and stability of Akt and protein kinase C. The EMBO journal 27, 1932-1943.

Folch, J., Lees, M., and Sloane Stanley, G. H. (1957). A simple method for the isolation and purification of total lipides from animal tissues. The Journal of biological chemistry 226, 497-509.

Ford, E. S., Giles, W. H., and Dietz, W. H. (2002). Prevalence of the metabolic syndrome among US adults: findings from the third National Health and Nutrition Examination Survey. JAMA: the journal of the American Medical Association 287, 356-359.

Gao, M. H., Miyanohara, A., Feramisco, J. R., and Tang, T. (2009). Activation of PH-domain leucine-rich protein phosphatase 2 (PHLPP2) by agonist stimulation in cardiac myocytes expressing adenylyl cyclase type 6. Biochemical and biophysical research communications 384, 193-198.

Gao, T., Furnari, F., and Newton, A. C. (2005). PHLPP: a phosphatase that directly dephosphorylates Akt, promotes apoptosis, and suppresses tumor growth. Molecular cell 18, 13-24.

Ghalali, A., Ye, Z. W., Hogberg, J., and Stenius, U. (2014). Phosphatase and Tensin Homolog Deleted on Chromosome 10 (PTEN) and PH Domain and Leucine-rich Repeat Phosphatase Cross-talk (PHLPP) in Cancer Cells and in Transforming Growth Factor beta-Activated Stem Cells. The Journal of biological chemistry 289, 11601-11615.

Haas, J. T., Miao, J., Chanda, D., Wang, Y., Zhao, E., Haas, M. E., Hirschey, M., Vaitheesvaran, B., Farese, R. V., Jr., Kurland, I. J., et al. (2012). Hepatic insulin signaling is required for obesity-dependent expression of SREBP-1c mRNA but not for feeding-dependent expression. Cell metabolism 15, 873-884.

Haeusler, R. A., et al. Integrated control of hepatic lipogenesis versus glucose production requires FoxO transcription factors. Nat Commun 5, 5190 (2014).

Hagiwara, A., Cornu, M., Cybulski, N., Polak, P., Betz, C., Trapani, F., Terracciano, L., Heim, M. H., Ruegg, M. A., and Hall, M. N. (2012). Hepatic mTORC2 activates glycolysis and lipogenesis through Akt, glucokinase, and SREBP1c. Cell metabolism 15, 725-738.

Han, S., Liang, C. P., Westerterp, M., Senokuchi, T., Welch, C. L., Wang, Q., Matsumoto, M., Accili, D., and Tall, A. R. (2009). Hepatic insulin signaling regulates VLDL secretion and atherogenesis in mice. The Journal of clinical investigation 119, 1029-1041.

Hands, S. L., Proud, C. G., and Wyttenbach, A. (2009). mTOR's role in ageing: protein synthesis or autophagy? Aging 1, 586-597.

Harrington, L. S., Findlay, G. M., Gray, A., Tolkacheva, T., Wigfield, S., Rebholz, H., Barnett, J., Leslie, N. R., Cheng, S., Shepherd, P. R., et al. (2004). The TSC1-2 tumor suppressor controls insulin-PI3K signaling via regulation of IRS proteins. The Journal of cell biology 166, 213-223.

Howell, J. J. & Manning, B. D. mTOR couples cellular nutrient sensing to organismal metabolic homeostasis. Trends in endocrinology and metabolism: TEM 22, 94-102 (2011).

Frescas, D., and Pagano, M. (2008). Deregulated proteolysis by the F-box proteins SKP2 and beta-TrCP: tipping the scales of cancer. Nature reviews Cancer 8, 438-449.

Ikenoue, T., Inoki, K., Yang, Q., Zhou, X., and Guan, K. L. (2008). Essential function of TORC2 in PKC and Akt turn motif phosphorylation, maturation and signaling. The EMBO journal 27, 1919-1931.

Jain, A., Arauz, E., Aggarwal, V., Ikon, N., Chen, J., and Ha, T. (2014). Stoichiometry and assembly of mTOR complexes revealed by single-molecule pulldown. Proceedings of the National Academy of Sciences of the United States of America 111, 17833-17838.

Kaizuka, T., Hara, T., Oshiro, N., Kikkawa, U., Yonezawa, K., Takehana, K., Iemura, S., Natsume, T., and Mizushima, N. (2010). Tti1 and Tel2 are critical factors in mammalian target of rapamycin complex assembly. The Journal of biological chemistry 285, 20109-20116.

Kim, D. H., Sarbassov, D. D., Ali, S. M., King, J. E., Latek, R. R., Erdjument-Bromage, H., Tempst, P., and Sabatini, D. M. (2002). mTOR interacts with raptor to form a nutrient-sensitive complex that signals to the cell growth machinery. Cell 110, 163-175.

Kim, D. H., Sarbassov, D. D., Ali, S. M., Latek, R. R., Guntur, K. V., Erdjument-Bromage, H., Tempst, P., and Sabatini, D. M. (2003). GbetaL, a positive regulator of the rapamycin-sensitive pathway required for the nutrient-sensitive interaction between raptor and mTOR. Molecular cell 11, 895-904.

Kim, K., Pyo, S., and Um, S. H. (2012). S6 kinase 2 deficiency enhances ketone body production and increases peroxisome proliferator-activated receptor alpha activity in the liver. Hepatology (Baltimore, Md.) 55, 1727-1737.

Kim, D. H., et al. GbetaL, a positive regulator of the rapamycin-sensitive pathway required for the nutrient-sensitive interaction between raptor and mTOR. Molecular cell 11, 895-904 (2003).

Kim, S., et al. Amino acid signaling to mTOR mediated by inositol polyphosphate multikinase. Cell metabolism 13, 215-221 (2011).

Leslie, N. R., Biondi, R. M., and Alessi, D. R. (2001). Phosphoinositide-regulated kinases and phosphoinositide phosphatases. Chemical reviews 101, 2365-2380.

Li, X., Liu, J. & Gao, T. beta-TrCP-mediated ubiquitination and degradation of PHLPP1 are negatively regulated by Akt. Molecular and cellular biology 29, 6192-6205 (2009).

Li, L., et al. SCD1 Expression is dispensable for hepatocarcinogenesis induced by AKT and Ras oncogenes in mice. PloS one 8, e75104 (2013).

Li, L., Wang, C., Calvisi, D. F., Evert, M., Pilo, M. G., Jiang, L., Yuneva, M., and Chen, X. (2013). SCD1 Expression is dispensable for hepatocarcinogenesis induced by AKT and Ras oncogenes in mice. PloS one 8, e75104.

Li, S., Brown, M. S., and Goldstein, J. L. (2010). Bifurcation of insulin signaling pathway in rat liver: mTORC1 required for stimulation of lipogenesis, but not inhibition of gluconeogenesis. Proceedings of the National Academy of Sciences of the United States of America 107, 3441-3446.

Li, X., Liu, J., and Gao, T. (2009). beta-TrCP-mediated ubiquitination and degradation of PHLPP1 are negatively regulated by Akt. Molecular and cellular biology 29, 6192-6205.

Li, X., Stevens, P. D., Liu, J., Yang, H., Wang, W., Wang, C., Zeng, Z., Schmidt, M. D., Yang, M., Lee, E. Y., et al. (2014). PHLPP Is a Negative Regulator of RAF1, Which Reduces Colorectal Cancer Cell Motility and Prevents Tumor Progression in Mice. Gastroenterology 146, 1301-1312.e1310.

Lin, H. V., and Accili, D. (2011). Hormonal regulation of hepatic glucose production in health and disease. Cell metabolism 14, 9-19.

Lu, M., et al. Insulin regulates liver metabolism in vivo in the absence of hepatic Akt and Foxo1. Nature medicine 18, 388-395 (2012).

Li X., Liu J. and Gao T. (2009). beta-TrCP-mediated ubiquitination and degradation of PHLPP1 are negatively regulated by Akt. Molecular and Cellular Biology 29(23), 6192-6205.

Lu, M., Wan, M., Leavens, K. F., Chu, Q., Monks, B. R., Fernandez, S., Ahima, R. S., Ueki, K., Kahn, C. R., and Birnbaum, M. J. (2012). Insulin regulates liver metabolism in vivo in the absence of hepatic Akt and Foxo1. Nature medicine 18, 388-395.

Menon, S., Dibble, C. C., Talbott, G., Hoxhaj, G., Valvezan, A. J., Takahashi, H., Cantley, L. C., and Manning, B. D. (2014). Spatial control of the TSC complex integrates insulin and nutrient regulation of mTORC1 at the lysosome. Cell 156, 771-785.

Michael, M. D., et al. Loss of insulin signaling in hepatocytes leads to severe insulin resistance and progressive hepatic dysfunction. Molecular cell 6, 87-97 (2000).

Mishra, N., et al. Efficient hepatic delivery of drugs: novel strategies and their significance. Biomed Res Int 2013, 382184 (2013).

Miyamoto, S., Purcell, N. H., Smith, J. M., Gao, T., Whittaker, R., Huang, K., Castillo, R., Glembotski, C. C., Sussman, M. A., Newton, A. C., et al. (2010). PHLPP-1 negatively regulates Akt activity and survival in the heart. Circulation research 107, 476-484.

Mora, A., Lipina, C., Tronche, F., Sutherland, C. & Alessi, D. R. Deficiency of PDK1 in liver results in glucose intolerance, impairment of insulin-regulated gene expression and liver failure. The Biochemical journal 385, 639-648 (2005).

Newton, A. C., and Trotman, L. C. (2014). Turning off AKT: PHLPP as a drug target. Annual review of pharmacology and toxicology 54, 537-558.

Oh, W. J., Wu, C. C., Kim, S. J., Facchinetti, V., Julien, L. A., Finlan, M., Roux, P. P., Su, B., and Jacinto, E. (2010). mTORC2 can associate with ribosomes to promote cotranslational phosphorylation and stability of nascent Akt polypeptide. The EMBO journal 29, 3939-3951.

Ono, H., et al. Hepatic Akt activation induces marked hypoglycemia, hepatomegaly, and hypertriglyceridemia with sterol regulatory element binding protein involvement. Diabetes 52, 2905-2913 (2003).

Pajvani, U. B., Qiang, L., Kangsamaksin, T., Kitajewski, J., Ginsberg, H. N., and Accili, D. (2013). Inhibition of Notch uncouples Akt activation from hepatic lipid accumulation by decreasing mTorc1 stability. Nature medicine 19, 1054-1060.

Peterson, T. R., Sengupta, S. S., Harris, T. E., Carmack, A. E., Kang, S. A., Balderas, E., Guertin, D. A., Madden, K. L., Carpenter, A. E., Finck, B. N., et al. (2011). mTOR complex 1 regulates lipin 1 localization to control the SREBP pathway. Cell 146, 408-420.

Porstmann, T., Santos, C. R., Griffiths, B., Cully, M., Wu, M., Leevers, S., Griffiths, J. R., Chung, Y. L., and Schulze, A. (2008). SREBP activity is regulated by mTORC1 and contributes to Akt-dependent cell growth. Cell metabolism 8, 224-236.

Sarbassov, D. D., Guertin, D. A., Ali, S. M., and Sabatini, D. M. (2005). Phosphorylation and regulation of Akt/PKB by the rictor-mTOR complex. Science (New York, N.Y.) 307, 1098-1101.

Sengupta, S., Peterson, T. R., Laplante, M., Oh, S., and Sabatini, D. M. (2010). mTORC1 controls fasting-induced ketogenesis and its modulation by aging. Nature 468, 1100-1104.

Shah, O. J., Wang, Z., and Hunter, T. (2004). Inappropriate activation of the TSC/Rheb/mTOR/S6K cassette induces IRS1/2 depletion, insulin resistance, and cell survival deficiencies. Current biology: CB 14, 1650-1656.

Slawik, M., and Vidal-Puig, A. J. (2006). Lipotoxicity, overnutrition and energy metabolism in aging. Aging research reviews 5, 144-164.

Stephens, L., Anderson, K., Stokoe, D., Erdjument-Bromage, H., Painter, G. F., Holmes, A. B., Gaffney, P. R., Reese, C. B., McCormick, F., Tempst, P., et al. (1998). Protein kinase B kinases that mediate phosphatidylinositol 3,4,5-trisphosphate-dependent activation of protein kinase B. Science (New York, N.Y.)

Taniguchi, C. M., Emanuelli, B., and Kahn, C. R. (2006). Critical nodes in signaling pathways: insights into insulin action. Nature reviews Molecular cell biology 7, 85-96.

Villanova, N., Moscatiello, S., Ramilli, S., Bugianesi, E., Magalotti, D., Vanni, E., Zoli, M., and Marchesini, G. (2005). Endothelial dysfunction and cardiovascular risk profile in nonalcoholic fatty liver disease. Hepatology (Baltimore, Md.) 42, 473-480.

Wang, J., Cheung, A. T., Kolls, J. K., Starks, W. W., Martinez-Hernandez, A., Dietzen, D., and Bryer-Ash, M. (2001). Effects of adenovirus-mediated liver-selective overexpression of protein tyrosine phosphatase-1b on insulin sensitivity in vivo. Diabetes, obesity & metabolism 3, 367-380.

Wang, P., Zhou, Z., Hu, A., Ponte de Albuquerque, C., Zhou, Y., Hong, L., Sierecki, E., Ajiro, M., Kruhlak, M., Harris, C., et al. (2014). Both Decreased and Increased SRPK1 Levels Promote Cancer by Interfering with PHLPP-Mediated Dephosphorylation of Akt. Molecular cell 54, 378-391.

Yabe, D., Brown, M. S., and Goldstein, J. L. (2002). Insig-2, a second endoplasmic reticulum protein that binds SCAP and blocks export of sterol regulatory element-binding proteins. Proceedings of the National Academy of Sciences of the United States of America 99, 12753-12758.

Yabe, D., Komuro, R., Liang, G., Goldstein, J. L., and Brown, M. S. (2003). Liver-specific mRNA for Insig-2 down-regulated by insulin: implications for fatty acid synthesis. Proceedings of the National Academy of Sciences of the United States of America 100, 3155-3160.

Yecies, J. L., et al. Akt stimulates hepatic SREBP1c and lipogenesis through parallel mTORC1-dependent and independent pathways. Cell metabolism 14, 21-32 (2011).

Yecies, J. L., Zhang, H. H., Menon, S., Liu, S., Yecies, D., Lipovsky, A. I., Gorgun, C., Kwiatkowski, D. J., Hotamisligil, G. S., Lee, C. H., et al. (2011). Akt stimulates hepatic SREBP1c and lipogenesis through parallel mTORC1-dependent and independent pathways. Cell metabolism 14, 21-32.

Yip, C. K., Murata, K., Walz, T., Sabatini, D. M., and Kang, S. A. (2010). Structure of the human mTOR complex I and its implications for rapamycin inhibition. Molecular cell 38, 768-774.

Yuan, M., Pino, E., Wu, L., Kacergis, M., and Soukas, A. A. (2012). Identification of Akt-independent regulation of hepatic lipogenesis by mammalian target of rapamycin (mTOR) complex 2. The Journal of biological chemistry 287, 29579-29588.

Zhang, Y. L., Hernandez-Ono, A., Siri, P., Weisberg, S., Conlon, D., Graham, M. J., Crooke, R. M., Huang, L. S., and Ginsberg, H. N. (2006). Aberrant hepatic expression of PPARgamma2 stimulates hepatic lipogenesis in a mouse model of obesity, insulin resistance, dyslipidemia, and hepatic steatosis. The Journal of biological chemistry 281, 37603-37615.

1. A method of reducing a subject's hepatic and plasma triglyceride levels comprising administering to a subject in need thereof a pharmaceutical composition comprising a pharmaceutical carrier and (a) a compound that increases PHLPP2 in liver cells in an amount effective to reduce the subject's hepatic and plasma triglyceride levels,
(b) a compound that increases free Raptor in liver cells in an amount effective to reduce the subject's hepatic and plasma triglyceride levels,
(c) a compound that prevents PHLPP2 degradation in liver cells in an amount effective to reduce the subject's hepatic and plasma triglyceride levels, or
(d) a compound that inhibits Glucagon signaling in liver cells in an amount effective reduce the subject's hepatic and plasma triglyceride levels.

2. (canceled)

3. The method of claim 1, wherein the pharmaceutical composition increases Raptor expression, thereby increasing free Raptor in the liver cells.

4. The method of claim 1, wherein the pharmaceutical composition inhibits interaction of Raptor and mTORC1, thereby increasing free Raptor in the liver cells.

5. The method of claim 1, wherein the compound reduces the expression of at least one lipogenic gene, wherein the at least one lipogenic gene is Srebp1c, Fasn, Acc1, or Scd1.

6. (canceled)

7. The method of claim 1, wherein the subject is afflicted with a metabolic disease, cirrhosis or hepatocellular carcinoma.

8. The method of claim 1, wherein the pharmaceutical composition comprises a polynucleotide.

9. The method of claim 1, wherein the pharmaceutical composition is targeted to the liver of the subject.

10. The method of claim 7, wherein the metabolic disease is obesity, hypertriglyceridemia, hyperinsulinemia, Type 2 Diabetes, fatty liver disease, nonalcoholic fatty liver disease or nonalcoholic steatohepatitis.

11-16. (canceled)

17. The method of claim 1, wherein the subject is a human.

18. The method of claim 1, wherein the subject's hepatic or plasma triglyceride levels are >150 mg/dL.

19. The method of claim 1, wherein the subject's hepatic or plasma triglyceride levels are >500 mg/dL, about 200 to 499 mg/dL, or about 150 to 199 mg/dL.

20. The method of claim 1, wherein the subject's hepatic or plasma triglyceride levels are reduced by at least 20%, at least 25%, at least 30%, at least 35%, at least 40%, at least 45%, at least 50%, at least 55%, at least 60%, at least 65%, at least 70%, or at least 75%, relative to the level prior to the administration.

21. A process for determining the amount of free Raptor in a subject's liver comprising:
a. obtaining a biological sample comprising liver cells of the subject;
b. separating free Raptor and mTORC1-associated Raptor in the sample; and
c. determining the amount of free Raptor in the sample.

22. A process for diagnosing whether a subject is afflicted with decreased free Raptor comprising:
a) determining the amount of free Raptor in the subject according to the process of claim 21;
b) determining the amount of free Raptor in a reference subject according to the process of claim 21; and
d) diagnosing the subject to be afflicted with decreased free Raptor if the amount of free Raptor in step (a) is substantially decreased compared to the amount of free Raptor in step (b).

23. A method of treating a subject diagnosed to be afflicted with decreased free Raptor according to the process of claim 22 comprising reducing the subject's hepatic and plasma triglyceride levels according to the method of claim 1.

24. A method of reducing a subject's hepatic and plasma triglyceride levels comprising administering to a subject in need thereof a pharmaceutical composition comprising a pharmaceutical carrier and (a) a compound that increases PHLPP2 in liver cells in an amount effective to reduce the subject's hepatic and plasma triglyceride levels, or
(b) a pharmaceutical composition comprising a pharmaceutical carrier and a compound that increases free Raptor in liver cells in an amount effective to reduce the subject's hepatic and plasma triglyceride levels, and
wherein the pharmaceutical composition inhibits Glucagon signaling, thereby reducing PHLPP2 degradation.

25. (canceled)

26. The method of claim 1, wherein the pharmaceutical composition reduces β-TrCP-mediated degradation of PHLPP2, thereby increasing free Raptor in the liver cells.

27. The method of claim 1, wherein the pharmaceutical composition comprises a Notch antagonist.

28. The method of claim 27, wherein the Notch antagonist comprises a γ-secretase inhibitor, anti-DLL4 mAB, or a DLK peptide.

29. The method of claim 1, wherein the pharmaceutical composition decreases PHLPP2 phosphorylation at Serine 1119 or Serine 1210 residues, inhibits interaction of PHLPP2 and KCTD17, decreases Akt signaling, decreases Akt phosphorylation at Serine 473 residue, or prevents PHLPP2 degradation by inhibiting Glucagon signaling.

30-37. (canceled)
